(19) United States
(12) Patent Application Publication
(10) Pub. No.: US 2024/0254516 A1
(43) Pub. Date: Aug. 1, 2024

(54) COMPLEX FOR GENOME EDITING HAVING STABILITY AND FEW SIDE-EFFECTS, AND NUCLEIC ACID CODING SAME

(71) Applicant: National University Corporation Kobe University, Kobe (JP)

(72) Inventor: Keiji NISHIDA, Kobe (JP)

(73) Assignee: NATIONAL UNIVERSITY CORPORATION KOBE UNIVERSITY, Kobe (JP)

(21) Appl. No.: 18/634,640

(22) Filed: Apr. 12, 2024

Related U.S. Application Data
(63) Continuation-in-part of application No. 16/764,799, filed on May 15, 2020.

(30) Foreign Application Priority Data
Nov. 22, 2017 (JP) 2017-225221

(51) Int. Cl. C12N 15/90; C12N 9/22; C12N 9/78; C12N 15/11

(52) U.S. Cl. CPC C12N 15/907; C12N 9/22; C12N 9/78; C12N 15/11; C12N 15/90; C12Y 305/04; C12N 2310/20; C12N 2800/80

(57) ABSTRACT

The present invention provides a complex containing a nucleic acid sequence-recognizing module and a proteolysis tag, wherein the module is linked to the proteolysis tag, the module specifically binds to a target nucleotide sequence in a double stranded DNA, and the tag consists of (i) a peptide containing 3 hydrophobic amino acid residues at the C-terminal, or (ii) a peptide containing 3 amino acid residues at the C-terminal wherein at least a part of the amino acid residues is substituted by serine

### CROSS-REFERENCE TO RELATED APPLICATIONS

This patent application is a continuation of co-pending U.S. patent application Ser. No. 16/764,799, filed May 15, 2020, which is the U.S. national phase of International Patent Application No. PCT/JP2018/042915, filed on Nov. 21, 2018, which claims the benefit of Japanese Patent Application No. 2017-225221 filed on Nov. 22, 2017, which are incorporated by reference in their entireties herein.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 445, 166 bytes Extensible Markup Language (xml) file named “770898-Sequence Listing.xml,” created Apr. 12, 2024.

### TECHNICAL FIELD

The present invention relates to a complex for genome editing which is stable and causes few side effects, a nucleic acid encoding same, and a genome editing method using the complex.

### BACKGROUND ART

Genome editing that does not require incorporation of a selection marker gene and can minimize the effect on expression of downstream genes in the same operon is particularly advantageous in prokaryotes. Phage-derived RecET and A-Red recombinases have been used as recombinant techniques and facilitate homology-dependent incorporation/substitution of donor DNA or oligonucleotides (e.g., non-patent literature 1). By combining with a strain deficient in methyl-directed mismatch repair (MMR), highly efficient recombination can be achieved without incorporating a selectable marker (non-patent literature 2), and genetic diversity at multiple target loci can be achieved within several days. Thus, the technique is utilized in multiplex automated genome engineering (MAGE). However, the aforementioned recombination technique relies on MMR deficiency and host-dependent factors such as RecA, which is a central constituent element of the recombinant DNA repair system, and damages most Escherichia coli used as a host for cloning. Therefore, it cannot be easily applied to bacterial species with a different background (non-patent literature 3).

CRISPR (clustered regularly interspaced short palindromic repeats) and CRISPR-associated (Cas) protein are known to work as a bacterial adaptive immune system by cleaving target DNA in a manner dependent on a single guide RNA (sgRNA) and protospacer adjacent motif (PAM). Cas9 nuclease from Streptococcus pyogenes is widely used as a powerful genome editing tool in eukaryotes having a double-stranded DNA break (DSB) repair pathway (e.g., non-patent literatures 4, 5). During the repair of DSB by the non-homologous end joining (NHEJ) pathway, a small insertion and/or deletion (indels) are/is introduced into the target DNA, and site-specific mutation or gene destruction occurs. Even though the efficiency depends on the host cell, homologous recombination repair (HDR) can be promoted by providing a donor DNA containing a homology arm to the target region for more accurate editing.

However, since the genome editing technique at present relies on the DNA repair system of the host, application to prokaryotes requires further designing. In most bacteria, DNA cleavage by artificial nucleases results in cell death due to the lack of the NHEJ pathway (non-patent literatures 6, 7). Therefore, CRISPR/Cas9 is used only as a counter-selector for cells with altered genes in other methods, such as the λ-Red recombination system (e.g., non-patent literatures 8, 9).

Recently, deaminase-mediated target base editing has been demonstrated in which nucleotides are directly edited at the target gene locus without using donor DNA containing a homology arm for the target region (e.g., patent literature 1, non-patent literatures 10-12). Since this technique utilizes DNA deamination instead of nuclease-mediated DNA cleavage, it does not induce bacterial cell death and is applicable to genome editing of bacteria. However, its mutation efficiency, especially the efficiency of simultaneous editing of multiple sites, is not sufficient.

### CITATION LIST

### Patent Literature

patent literature 1: WO 2015/133554

### Non-Patent Literature

non-patent literature 1: Datsenko, K. A. & Wanner, B. L., Proc. Natl. Acad. Sci. U.S.A 97, 6640-5 (2000).
non-patent literature 2: Costantino, N. & Court, D. L., Proc. Natl. Acad. Sci. U.S.A 100, 15748-53 (2003).
non-patent literature 3: Wang, J. et al., Mol. Biotechnol. 32, 43-53 (2006).
non-patent literature 4: Mali, P. et al., Science 339, 823-827 (2013).
non-patent literature 5: Cong, L. et al., Science 339, 819-823 (2013).
non-patent literature 6: Bowater, R. & Doherty, A. J., PLOS Genet. 2, 93-99 (2006).
non-patent literature 7: Cui, L. & Bikard, D., Nucleic Acids Res. 44, 4243-4251 (2016).
non-patent literature 8: Jiang, W. et al., Nat Biotechnol 31, 233-239 (2013).
non-patent literature 9: Li, Y. et al., Metab. Eng. 31, 1-9 (2015).
non-patent literature 10: Komor, A. C. et al., Nature 61, 5985-91 (2016).
non-patent literature 11: Nishida, K. et al., Science 102, 553-563 (2016).
non-patent literature 12: Ma, Y. et al., Nat. Methods 1-9 (2016). doi: 10.1038/nmeth.4027

### SUMMARY OF INVENTION

### Technical Problem

Conventional vectors for genome editing impose heavy burden on hosts, particularly bacteria, and may make the vector unstable in the host, due to a high toxicity of the complex for genome editing that is expressed from the vector and acts on the genomic DNA of the host. In genome editing, side effects such as non-specific mutation, off-target mutation and the like occur. Particularly, when the mutation efficiency is increased using uracil DNA glycosylase inhibitor (UGI) and the like, a strong toxicity to the host occurs in response, and cell death, an increase in the non-specific mutation rate and the like occur. Therefore, an object of the present invention is to provide a nucleic acid such as a vector having low toxicity that can be stably amplified even in a host, and a complex for genome editing encoded by the nucleic acid, and a method for genome editing using the vector, and a nucleic acid altering enzyme as necessary, which method does not rely on host-dependent factors such as RecA, can alter DNA of bacterium while suppressing non-specific mutation and the like, and is applicable to a wide range of bacteria.

### Solution to Problem

The present inventor had an idea that a vector in a bacterium may be stabilized and non-specific mutation of bacterial DNA and the like may be reduced by suppressing the amount of a complex for genome editing present in the bacterium which has high toxicity to the bacterium as a host. To suppress the amount of a complex for genome editing, the inventor took note of LVA tag which is a proteolysis tag known to promote degradation of proteins in bacteria and shorten the half-life, and proceeded with the research. As a result, the inventor has demonstrated that non-specific mutation can be reduced while maintaining mutation efficiency into the target site by adding a proteolysis tag to a complex for genome editing and that even when UGI is combined, non-specific mutation can be reduced and the target sequence can be altered with high efficiency (FIG. 9, FIG. 10). The present inventor conducted further studies and completed the present invention.

Accordingly, the present invention provides the following.
[1] A complex comprising a nucleic acid sequence-recognizing module and a proteolysis tag, wherein the module is linked to the proteolysis tag, the module specifically binds to a target nucleotide sequence in a double stranded DNA, and the tag consists of (i) a peptide containing 3 hydrophobic amino acid residues at the C-terminal, or (ii) a peptide containing 3 amino acid residues at the C-terminal wherein at least a part of the amino acid residues is substituted by serine.
[2] The complex of [1], wherein the aforementioned complex is further bound with a nucleic acid altering enzyme, and converts one or more nucleotides in the targeted site to other one or more nucleotides or deletes them, or insert one or more nucleotides in the targeted site.
[3] The complex of [1] or [2], wherein the aforementioned 3 amino acid residues are leucine-valine-alanine, leucine-alanine-alanine, alanine-alanine-valine or alanine-serine-valine.
[4] The complex of any of [1] to [3], wherein the aforementioned nucleic acid sequence-recognizing module is a CRISPR-Cas system in which only one of the two DNA cleavage abilities of Cas or both DNA cleavage abilities are inactivated.
[5] The complex of any of [1] to [3], wherein the aforementioned complex is a complex in which the proteolysis tag is bound to a CRISPR-Cas system.
[6] The complex of any of [2] to [4], wherein the aforementioned nucleic acid altering enzyme is a nucleic acid base converting enzyme or a DNA glycosylase.
[7] The complex of [6], wherein the aforementioned nucleic acid base converting enzyme is deaminase.
[8] The complex of [6] or [7], wherein a base excision repair inhibitor is further bound to the complex.
[9] A nucleic acid encoding the complex of any of [1] to [8].
[10] A method for altering a targeted site of a double stranded DNA of a bacterium, or regulating an expression of a gene encoded by a double-stranded DNA near the site, comprising a step of bringing a complex comprising a nucleic acid sequence-recognizing module specifically binding to a target nucleotide sequence in a selected double stranded DNA and a proteolysis tag, wherein the proteolysis tag consists of (i) a peptide containing 3 hydrophobic amino acid residues at the C-terminal, or (ii) a peptide containing 3 amino acid residues at the C-terminal wherein at least a part of the amino acid residues is substituted by serine into contact with the double stranded DNA.
[11] The method of [10], comprising a step of converting one or more nucleotides in the targeted site to other one or more nucleotides or delete one or more nucleotides, or inserting one or more nucleotides into said targeted site, wherein the aforementioned complex is further bound with a nucleic acid altering enzyme.
[12] The method of [10] or [11], wherein the aforementioned 3 amino acid residues are leucine-valine-alanine, leucine-alanine-alanine, alanine-alanine-valine or alanine-serine-valine.
[13] The method of any of to [12], wherein the aforementioned nucleic acid sequence-recognizing module is a CRISPR-Cas system in which only one of the two DNA cleavage abilities of Cas or both DNA cleavage abilities are inactivated.
[14] The method of any of [10] to [12], wherein the aforementioned complex is a complex in which the proteolysis tag is bound to a CRISPR-Cas system.
[15] The method of any of [10] to [14], wherein two or more kinds of nucleic acid sequence-recognizing modules each specifically binding to a different target nucleotide sequence are used.
[16] The method of [15], wherein the aforementioned different target nucleotide sequence is present in a different gene.
[17] The method of any of [10] to [13], [15] and [16], wherein the aforementioned nucleic acid altering enzyme is a nucleic acid base converting enzyme or a DNA glycosylase.
[18] The method of [17], wherein the aforementioned nucleic acid base converting enzyme is deaminase.
[19] The method of or [18], wherein the complex is further bound with a base excision repair inhibitor.
[20] The method of any of [10] to [19], wherein the double stranded DNA is brought into contact with the complex by introduction the nucleic acid encoding the complex into the bacterium having the double stranded DNA.

### Advantageous Effects of Invention

According to the present invention, a nucleic acid (e.g., vector) which is stable and amplifiable even in a host bacterium and has low toxicity, and a complex for genome editing which is encoded by the nucleic acid are provided. According to the method for genome editing using the nucleic acid and nucleic acid altering enzyme of the present invention, it is possible to alter the gene of a host bacterium while suppressing non-specific mutation and the like, or regulate the expression of a gene encoded by a double stranded DNA. Since this method does not rely on host dependent factors such as RecA, it can be applied to a wide range of bacteria.

### BRIEF DESCRIPTION OF DRAWINGS

FIG. 1 shows the outline of the Target-AID system in a bacterium. (a) is a schematic model of Target-AID (dCas9-PmCDA1/sgRNA) base editing. dCas9-PmCDA1/sgRNA complex binds to the double-stranded DNA to form an R-loop in sgRNA-and PAM-dependent manner. PmCDA1 catalyzes deamination of cytosine located at a top (non-complementary) strand within 15-20 bases upstream from PAM, which results in C-to-T mutagenesis. (b) shows a single Target-AID plasmid of bacterium. The plasmid contains the chloramphenicol-resistant (Cm^{R}) gene, temperature-sensitive (ts) λ cI repressor, the pSC101 origin (ori) and RepA101 (ts). λ operator expresses dCas9-PmCDA1 fusion at high temperature (>37° c.) as cI repressor (ts) gets inactivated. sgRNA is expressed by a constitutive promoter J23119. dCas9 is a nuclease-deficient Cas9 with D10A H840A mutations, and PmCDA1 is Petromyzon marinus cytosine deaminase.

FIG. 2 shows the transformation efficiency of the Cas9 and Target-AID vectors in Escherichia coli. Plasmids expressing each protein for alteration (Cas9, dCas, Cas9-CDA, nCas-CDA or dCas-CDA) with sgRNA targeting galK gene were transformed into Escherichia coli DH5α strain and selected for chloramphenicol resistance marker. Viable cells were counted and calculated as colony forming unit (CFU) per amount of transformed plasmid DNA. Dots represent three independent experiments and box indicates 95% confidence interval for a geometric mean by t-test analysis.

FIG. 3 shows a mutation induced in a specific site of the galK9 gene (5′ to 3′ is SEQ ID NO: 202, 3′ to 5′ is SEQ ID NO: 203, and amino acid translation below is SEQ ID NO: 204) by dCas-CDA. DH5α cells expressing dCas-CDA with galK_9 targeting sgRNA were spotted onto the LB agar plate to isolate single colonies. Randomly selected eight clones were sequenced and sequences were aligned. Translated amino acid sequences are shown on the bottom of each nucleotide sequences. Frequency of the aligned sequence is indicated as clone count. Box and inverted box indicate target sequence and PAM sequence, respectively. ORF number is indicated on the top. Mutated sites are highlighted in black shade and mutated bases and amino acids are shown in bold. Mutated codons are underlined. The clones in descending order are SEQ ID NO: 205 (amino acid translation SEQ ID NO: 207), SEQ ID NO: 206 (amino acid translation SEQ ID NO: 207), and SEQ ID NO: 202 (amino acid translation SEQ ID NO: 204).

FIG. 4 shows the mutation frequency assessed by drug resistance. (a) shows the galK mutagenesis and 2-DOG-resistance frequency. DH5α cells expressing dCas-CDA with non-targeting sgRNA (vector) or galK_9 target ssRNA were spotted onto the M63 medium agar plate with or without 2-DOG in serial dilutions, and colonies were counted. (b) shows the rpoB mutagenesis and rifampicin-resistance frequency. Cells expressing dCas-CDA with non-targeting sgRNA (vector) or rpoB_1 target ssRNA were spotted onto the LB agar plate with or without rifampicin in serial dilutions, and colonies were counted. Drug resistance frequency is calculated as a number of drug resistant colonies over that of non-selected colonies. Dots represent four independent experiments and box indicates 95% confidence interval for a geometric mean by t-test analysis.

FIG. 5 shows the gain-of-function mutagenesis of rpoB gene. (a) shows the sequence alignment of rpoB mutations induced by dCas-CDA. DH5α cells expressing dCas-CDA with rpoB_1 targeting sgRNA were spotted onto the LB agar plate to isolate single colonies. Randomly selected eight clones were sequenced and sequences were aligned. Translated amino acid sequences are shown on the bottom of each nucleotide sequences. Frequency of the aligned sequences is indicated as clone count. Box and inverted box indicate target sequence and PAM sequence. ORF number is indicated on the top. Mutated sites are highlighted in black shade and mutated bases and amino acids are shown in bold. Mutated codons are underlined. The rpoB_1 gene 5′ to 3′ is SEQ ID NO: 208. The rpoB 1 gene 3′ to 5′ is SEQ ID NO: 209. The amino acid translation of the rpoB_1 gene is SEQ ID NO: 210. The top clone is SEQ ID NO: 211, and the amino acid translation is SEQ ID NO: 212. The bottom clone is SEQ ID NO: 213, and the amino acid translation is SEQ ID NO: 214. (b) shows the results of the whole genome sequencing analyses of rpoB-targeted cells. Rifampicin-selected independent three clones were subjected to whole genome sequencing. Sequence coverage was calculated as sum base pair of mapped sequence over 4, 631 Mbp of Escherichia coli BW25113 genome sequence. Parental/variable mutation is shown by the number of variants obtained by subtracting common parental mutations from detected variants over 50% frequency including insertions, deletions, single nucleotide variants (SNV) and multiple nucleotide variants (MNV). Detected mutation indicates the number of mutations (count), genomic locus (region/gene), reference genome sequence (reference) and mutant allele (allele). Variant calling was performed as described in Examples. (c) shows the sequences around detected mutations listed in (b). Mutated sites are hi ghlighted in gray shade and mutated bases and amino acid are highlighted in bold. yne0 is SEQ ID NO: 215. gatC is SEQ ID NO: 216. yihN is SEQ ID NO: 217.

FIG. 6 shows the presence or absence of UGI-LVA, and mutation position and frequency when sgRNA with a different length was used. Target sequences (galK_8, 9, 11 and 13) with 20 nt length and longer were tested by using dCas-CDA (blue bar) or dCas-CDA-UGI-LVA (red bar) and analyzed by deep sequencing. Average of three independent experiments was plotted. Gray shade and inverted box indicate galk targeting sequences and PAM, respectively. Mutated bases are underlined. galk_8 is SEQ ID NO: 218, galK_9 is SEQ ID NO: 222, galK_11 is SEQ ID NO: 220, and galK_13 is SEQ ID NO: 224.

FIG. 7 shows the effect of target sequence properties on mutational positions and frequencies for Target-AID. Cells expressing dCas-CDA and each targeting sgRNA were analyzed by deep sequencing. Targeting sequences (20nt length or as indicated) were (+) DNA strand on galK ORF or (−) DNA strand below galK ORF, and expectedly introduced missense (M) or nonsense (N) mutations. Corresponding ORF number is indicated (Position). Mutation frequencies of the peak base position (highlighted in gray shade in the sequence) were obtained as averages of three independent experiments. Mutation frequencies in>50%, 10-50% or <10% are highlighted in shades of gray. The sequence (5′->3′) of galk_1 is SEQ ID NO: 226, galK_2 is SEQ ID NO: 227, galK_3 is SEQ ID NO: 228, galK_4 is SEQ ID NO: 229, galK_5 is SEQ ID NO: 230, galK_6 is SEQ ID NO: 231, galK_7 is SEQ ID NO: 232, galK_8 [20 nt] is SEQ ID NO: 233, galK_8 [21 nt] is SEQ ID NO: 234, galK_9 [20 nt] is SEQ ID NO: 235, galK_9 [21 nt] is SEQ ID NO: 236, galK_10 is SEQ ID NO: 237, galK_11 [20 nt] is SEQ ID NO: 238, galK_11 [23 nt] is SEQ ID NO: 239, galK_12 is SEQ ID NO: 240, galK_13 [20 nt] is SEQ ID NO: 241, galK_13 [21 nt] is SEQ ID NO: 242, and galK_14 is SEQ ID NO: 243.

FIG. 8 shows the Effect of target lengths on the mutational spectrum. (a) shows the mutation frequencies using various lengths of target sequences in the gsiA. Target sequences that contain poly-C on the distal site was edited by dCas-CDA-UGI-LVA and analyzed by deep sequencing. Mutational spectra for sgRNAs with 18nt, 20nt, 22nt or 24nt length were distinguished by the shade of gray. Averages for three independent experiments are shown. Inverted box indicates PAM. Mutated bases are underlined. The sequences in FIG. 8A top to bottom are SEQ ID NO: 244, SEQ ID NO: 245 and SEQ ID NO: 246. (b) shows the mutation frequencies for targets in ychF (5′->3′ is SEQ ID NO: 247 and 3′ to 5′ is SEQ ID NO: 248) and yfiH (5′->3′ is SEQ ID NO: 249 and 3′->5′ is SEQ ID NO: 250). Targets are set on the bottom strands. Mutational spectra for sgRNAs with 18nt, 20nt or 22nt length are used and shown as in (a). (c) shows averaged mutational spectra for each sgRNA length of (a) and (b). Peak positions are numbered.

FIG. 9 shows the multiple mutagenesis in the gal gene. (a) shows the non-specific mutagenic effect assessed by rifampicin resistance. Cells expressing each protein (vector, dCas, dCas-CDA, dCas-CDA-LVA or dCas-CDA1-UGI-LVA) with tandem-sgRNA-unit containing galk_10-galK_11-galK_13 targets were spotted onto the LB agar plate with or without rifampicin to assess the frequency of non-specific mutations. Dots represent at least three independent experiments and box indicates 95% confidence interval for a geometric mean by t-test analysis. (b) shows frequency of on-target multiplex mutation induced in the target region. Randomly selected eight clones in (a) were sequenced at the targeted three loci. Frequencies of the single-, double- or triple-mutant clone are indicated. (c) and (d) show the sequence alignments of the mutants. Single targets (galK_10, galK_11 or galk_13) (c) or triple targets (d) were mutated using dCas-CDA-UGI-LVA. Randomly selected eight clones were sequenced and sequences were aligned. Box and inverted box indicate target sequence and PAM sequence, respectively. Mutation sites and mutated bases are highlighted in black shade and bold, respectively. In (c), galK_10 is 5′ to 3′ SEQ ID NO: 251 and 3′ to 5′ SEQ ID NO: 252, and the galK_10 clone is SEQ ID NO: 253. galK_11 is 5′ to 3′ SEQ ID NO: 254 and 3′ to 5′ SEQ ID NO: 255, and the galK_11 clone is SEQ ID NO: 256. galK_13 is 5′ to 3′ SEQ ID NO: 224 and 3′ to 5′ SEQ ID NO: 258, and the galK_13 clone is SEQ ID NO: 259. In (d), the combined galK_10 and galK_11 is 5′ to 3′ SEQ ID NO: 331 and 3′ to 5′ SEQ ID NO: 332. The combined galk_10 and galK_11 clones from top to bottom are SEQ ID NO: 333, SEQ ID NO: 334, SEQ ID NO: 335, SEQ DI NO: 336, SEQ ID NO: 336, SEQ ID NO: 337, SEQ ID NO: 337, and SEQ ID NO: 331. galK_13 is 5′ to 3′ SEQ ID NO: 262 and 3′ to 5′ SEQ ID NO: 263. The galK_13 clones from top to bottom are SEQ ID NO: 269, SEQ ID NO: 269, SEQ ID NO: 270, SEQ ID NO: 269, SEQ ID NO: 270, SEQ ID NO: 269, SEQ ID NO: 269, and SEQ ID NO: 262.

FIG. 10 shows the multiplex mutagenesis. (a) shows a schematic drawing of two plasmids for multiplex mutagenesis; a vector for alteration expressing dCas-CDA-UGI-LVA and a plasmid pSBP80608 containing two tandem-repeated sgRNA-units containing three targeting sgRNA. (b) shows the sequence alignments of the targeted regions. Randomly selected eight clones were sequenced and aligned at each targeted region. Clone numbers are indicated on the left of the sequences. Box and inverted box indicate targeted sequence and PAM. Mutated sites and bases are highlighted in black shade and bold. galK_2 is 5′ to 3′ SEQ ID NO: 271 and 3′ to 5′ SEQ ID NO: 272. The galK_2 clones are from 1-8, SEQ ID NO: 273, SEQ ID NO: 273, SEQ ID NO: 274, SEQ ID NO: 271, SEQ ID NO: 275, SEQ ID NO: 276, SEQ ID NO: 276, and SEQ ID NO: 276, respectively. xylB_1 is 5′ to 3′ SEQ ID NO: 277 and from 3′ to 5′ SEQ ID NO: 278. The xylB_1 clones are from 1-8, SEQ ID NO: 279, SEQ ID NO: 282, SEQ ID NO: 281, SEQ ID NO: 277, SEQ ID NO: 282, SEQ ID NO: 281, SEQ ID NO: 283, and SEQ ID NO: 282, respectively. manA 1 is 5′ to 3′ SEQ ID NO: 285 and 3′ to 5′ SEQ ID NO: 285. The manA_1 clones are from 1-8, SEQ ID NO: 286, SEQ ID No: 286, SEQ ID NO: 287, SEQ ID NO: 284, SEQ ID NO: 286, SEQ ID NO: 288, SEQ ID NO: 286. pta_1 is 5′ to 3′ SEQ ID NO: 289 and 3′ to 5′ SEQ ID NO: 290. The pta_1 clones from 1-8 are SEQ ID NO: 291, SEQ ID NO: 291 SEQ ID NO: 291, SEQ ID NO: 289, SEEQ ID NO: 291, SEQ ID NO: 291, SEQ ID NO: 291, and SEQ ID NO: 291, respectively. adhE_3 is 5′ to 3′ SEQ ID NO: 292 and 3′ to 5′ SEQ ID NO: 293. The adhE_3 clones are from 1-8, SEQ ID NO: 338, SEQ ID NO: 294, SEQ ID NO: 294, SEQ ID NO: 292, SEQ ID NO: 294, SEQ ID NO: 296, SEQ ID NO: 297, AND SEQ ID NO: 294. tpiA_2 is 5′ to 3′ SEQ ID NO: 298 and 3′ to 5′ SEQ ID NO: 299. The tpiA_2 clones are from 1-8, SEQ ID NO: 300, SEQ ID NO: 300, SEQ ID NO: 300, SEQ ID NO: 298, SEQ ID NO: 300, SEQ ID NO: 300, SEQ ID NO: 300, and SEQ ID NO: 300, respectively.

FIG. 11 shows the simultaneous disruption of multi-copy transposase genes. IS1, 2, 3 and 5 are simultaneously targeted using dCas-CDA-UGI-LVA. sgRNAs are designed to introduce stop codons in the common sequence of the same type of transposase. All but the sequences which cannot be amplified from DH10B reference genome are aligned. Translated amino acid sequences are shown on the top of each common sequence. Genomic regions of each sequence are shown on the left. All target sequences are designed on the complementary strands and corresponding regions are squared with complementary PAM sequences (inverted). Mutated bases are highlighted in black shade. The IS1 targeted sequence is SEQ ID NO: 301 and the amino acid translation is SEQ ID NO: 302. 1-1 is SEQ ID NO: 303, 1-3 is SEQ ID NO: 304, 1-5 is SEQ ID NO: 339, 1-6 is SEQ ID NO: 304, 1-7 is SEQ ID NO: 305, 1-8 is SEQ ID NO: 304, 1-9 is SEQ ID NO: 304, 1-10 is SEQ ID NO: 303, 1-11 is SEQ ID NO: 303, and 1-12 is SEQ ID NO: 304. The IS2 targeted sequence is SEQ ID NO: 306 and the amino acid translation is SEQ ID NO: 307. 2-1 is SEQ ID NO: 308, 2-2 is SEQ ID NO: 309, 2-4 is SEQ ID NO: 308, 2-5 is SEQ ID NO: 310, 2-6 is SEQ ID NO: 308, 2-7 is SEQ ID NO: 308, 2-8 is SEQ ID NO: 308, 2-9 is SEQ ID NO: 308, 2-10 is SEQ ID NO: 308, 2-11 is SEQ ID NO: 308, 2-12 is SEQ ID NO: 311, and 2-13 is SEQ ID NO: 312. The IS3 targeted sequence is SEQ ID NO: 313 and the amino acid translation is SEQ ID NO: 314. 3-1 is SEQ ID NO: 340, 3-2 is SEQ ID NO: 340, 3-3 is SEQ ID NO: 315, 3-4 is SEQ ID NO: 317, and 3-5 is SEQ ID NO: 315. The IS5 targeted sequence is SEQ ID NO: 318 and the translation is SEQ ID NO: 319. 5-1 is SEQ ID NO: 320, 5-2 is SEQ ID NO: 320, 5-3 is SEQ ID NO: 321, 5-4 is SEQ ID NO: 320, 5-5 is SEQ ID NO: 322, 5-6 is SEQ ID NO: 320, 5-7 is SEQ ID NO: 322, 5-8 is SEQ ID NO: 322, 5-9 is SEQ ID NO: 320, 5-11 is SEQ ID NO: 322, 5-12 is SEQ ID NO: 323, 5-13 is SEQ ID NO: 320, 5-14 is SEQ ID NO: 320, and 5-15 is SEQ ID NO: 320.

FIG. 12 shows an isolation and verification procedure for IS-edited cells. Clone isolation and sequence verification as done in stages. Isolated clones are numbered as indicated on the top row of each table and sequence-analyzed at the IS sites indicated on the left column. Genotype was determined as targeted mutation verified (mut), not mutated (wt) or hetero of mut and wt (hetero) based on the Sanger sequencing spectrum.

FIG. 13 is a schematic showing of the vectors for yeast expression (background: pRS315 vector) used in Example 5. In the Figure, Gallp is a GAL1-10 promoter.

### DESCRIPTION OF EMBODIMENTS

### 1. Complex for Genome Editing and Nucleic Acid Encoding Same

The present invention provides a complex for genome editing in which a nucleic acid sequence-recognizing module specifically binding to a target nucleotide sequence in a double stranded DNA and a proteolysis tag are linked, and a nucleic acid encoding the complex. In one embodiment of the complex for genome editing of the present invention, a complex wherein a nucleic acid altering enzyme is further linked (i.e., a complex in which a nucleic acid sequence-recognizing module, a nucleic acid altering enzyme and a proteolysis tag are linked), which can alter the nucleic acid in the targeted site is provided. In one embodiment, to improve the alteration efficiency of the double stranded DNA, a base excision repair inhibitor may be further linked to the complex. In another embodiment of the complex for genome editing of the present invention, a complex wherein at least a nucleic acid sequence-recognizing module and a proteolysis tag are linked, which can regulation the expression of a gene double stranded DNA in the vicinity of the targeted site, is provided. In one embodiment, a transcriptional regulatory factor may be further linked to the complex. In the following, a complex in which at least any of a nucleic acid altering enzyme, a base excision repair inhibitor and a transcriptional regulatory factor is linked and a complex in which none of them is linked are sometimes collectively referred to as “the complex of the present invention” or “the complex for genome editing”, and particularly, a complex in which a nucleic acid altering enzyme is linked is sometimes referred to as “the nucleic acid altering enzyme complex”. The nucleic acids encoding these complexes are sometimes collectively referred to as “the nucleic acid of the present invention”.

When the nucleic acid of the present invention is introduced into a host bacterium (e.g., Escherichia coli) and cultured for the purpose of replication, rather than alteration of DNA, and a complex is expressed unintendedly from the nucleic acid, the toxicity to host bacterium can be suppressed low because the complex is degraded rapidly by the proteolysis tag. In fact, when the nucleic acid of the present invention is introduced into a host bacterium for the purpose of replication of the nucleic acid, the transformation efficiency of the host bacterium is high as demonstrated in the Examples described below as compared to when one not containing a nucleic acid encoding proteolysis tag is introduced. Therefore, the nucleic acid of the present invention containing a sequence encoding proteolysis tag can be replicated stably in a bacterium as a nucleic acid for genome editing of a host other than bacteria (e.g., eukaryote). Therefore, it is useful to add a sequence encoding the proteolysis tag of the present invention to a vector aiming at genome editing in a host other than bacteria.

In the present invention, the “alteration” of a double stranded DNA means that a nucleotide (e.g., dC) on a DNA strand is converted to another nucleotide (e.g., dT, dA or dG), or deleted, or a nucleotide or a nucleotide sequence is inserted between certain nucleotides on a DNA strand. The double stranded DNA to be altered is not particularly limited as long as it is a DNA present in the cell, preferably a genomic DNA. The “targeted site” of a double stranded DNA means the whole or partial “target nucleotide sequence”, which a nucleic acid sequence-recognizing module specifically recognizes and binds to, or the vicinity of the target nucleotide sequence (one or both of 5′ upstream and 3′ downstream). The “target nucleotide sequence” means a sequence to which a nucleic acid sequence-recognizing module in the double stranded DNA binds. In the present invention, the term “genome editing” is used to mean not only altering a double stranded DNA but also promoting or suppressing the expression of a gene encoded by a double stranded DNA in the vicinity of the targeted site.

In the present invention, the “nucleic acid sequence-recognizing module” means a molecule or molecule complex having an ability to specifically recognize and bind to a particular nucleotide sequence (i.e., target nucleotide sequence) on a DNA strand. When a nucleic acid altering enzyme complex is used, binding of the nucleic acid sequence-recognizing module to a target nucleotide sequence enables a nucleic acid altering enzyme and/or a base excision repair inhibitor linked to the module to specifically act on a targeted site of a double stranded DNA.

In the present invention, the “nucleic acid altering enzyme” means an enzyme that modifies DNA, and the modification directly or indirectly causes alteration of DNA. The enzyme may be a peptide fragment thereof as long as it has a catalytic activity. Examples of such DNA modification reaction include a reaction to cleave DNA (hereinafter to be also referred to as “DNA strand cleavage reaction”) which is catalyzed by a nucleolytic enzyme, a reaction to convert a substituent on the purine or pyrimidine ring of a nucleic acid base to other group or atom, which is a reaction catalyzed by a nucleic acid base converting enzyme and not directly involving cleavage of DNA strand (hereinafter to be also referred to as “nucleic acid base conversion reaction”) (e.g., deamination reaction of base), a reaction to hydrolyze N-glycoside linkage of DNA (hereinafter to be also referred to as “base excision reaction”) which is catalyzed by DNA glycosylase and the like. As shown in the Examples described below, the toxicity of a nucleic acid altering enzyme complex containing a nucleic acid base converting enzyme to the host bacterium can be reduced by adding a proteolysis tag. Therefore, the technique of the present invention can be applied to genome editing using not only a nucleic acid base converting enzyme but also nucleolytic enzymes conventionally difficult for application to bacteria due to the strong toxicity thereof. Therefore, the nucleic acid altering enzyme to be used in the present invention includes a nucleolytic enzyme, a nucleic acid base converting enzyme, a DNA glycosylase and the like. From the aspect of reduction of cytotoxicity, nucleic acid base converting enzyme and DNA glycosylase are preferable, and the targeted site can be altered using these enzymes in the targeted site, without cleaving at least one the strands of the double stranded DNA.

In the present invention, the “proteolysis tag” mainly consists of a peptide containing not less than 3 hydrophobic amino acid residues, wherein the peptide shows a shortened half-life of protein when added to a complex for genome editing as compared to that without addition to the complex. As such amino acid, glycine, alanine, valine, leucine, isoleucine, methionine, proline, phenylalanine, and tryptophan can be mentioned. The proteolysis tag of the present invention only needs to contain any three of these amino acid residues at the C-terminal, and other constitution is not particularly limited. It may be a peptide consisting of the three amino acid residues. A peptide in which a part or all of the aforementioned hydrophobic amino acid residues is/are substituted by serine or threonine is also encompassed in the proteolysis tag of the present invention. While preferable aforementioned three amino acid residues are not particularly limited, leucine-valine-alanine (LVA), leucine-alanine-alanine (LAA), alanine-alanine-valine (AAV) and the like, whose high effects were acknowledged in Escherichia coli and Pseudomonas putida (Andersen J. B. et al., Apll. Environ. Microbiol., 64: 2240-2246 (1998)), can be mentioned and, as one containing serine, alanine-serine-valine (ASV) and the like can be mentioned. In addition, the data base of tm RNA tag peptide (e.g., tmRDB, ag.auburn.edu/mirror/tmRDB/peptide/peptidephylolist.html) and the like can be referred to for the proteolysis tag containing these three amino acid residues. Specifically, YAASV (SEQ ID NO: 324), YALAA (SEQ ID NO: 325), ANDENYALAA (SEQ ID NO: 181) and AANDENYALAA (SEQ ID NO: 182) known as tmRNA tag peptides of Escherichia coli, GKQNNLSLAA (SEQ ID NO: 183), GKSNNNFALAA (SEQ ID NO: 184), GKENNNFALAA (SEQ ID NO: 185), GKTNSFNONVALAA (SEQ ID NO: 186), GKSNONLALAA (SEQ ID NO: 187) and GKONYALAA (SEQ ID NO: 188) known as tmRNA tag peptides of genus Bacillus, ANDDNYALAA (SEQ ID NO: 189), ANDDQYGAALAA (SEQ ID NO: 190), ANDENYGQEFALAA (SEQ ID NO: 191), ANDETYGDYALAA (SEQ ID NO: 192), ANDETYGEYALAA (SEQ ID NO: 193), ANDETYGEETYALAA (SEQ ID NO: 194), ANDENYGAEYKLAA (SEQ ID NO: 195) and ANDENYGAQLAA (SEQ ID NO: 196) known as tmRNA tag peptides of genus Pseudomonas, AKNTNSYALAA (SEQ ID NO: 197), AKNTNSYAVAA (SEQ ID NO: 198), AKNNTTYALAA (SEQ ID NO: 199), AKNTNTYALAA (SEQ ID NO: 200) and AKNNTSYALAA (SEQ ID NO: 201) known as tmRNA tag peptides of genus Streptococcus and the like can be unlimitatively mentioned. The proteolysis tag typically consists of 3-15 amino acid residues, but is not limited to this range. In one embodiment, proteolysis tag consists of 3-5 amino acid residues. Those of ordinary skill in the art can appropriately select the proteolysis tag according to the kind of host bacterium and the like. In the present specification, unless otherwise specified, the capital letter of the alphabet indicates a one-letter code for the amino acid, and the amino acid sequence is indicated from left to right, from N-terminal to C-terminal.

In the present invention, the “complex for genome editing” means a molecular complex having nucleic acid alteration activity or expression regulation activity and imparted with a particular nucleotide sequence recognition, which includes the above-mentioned complex in which a nucleic acid sequence-recognizing module and a proteolysis tag are linked. The “nucleic acid altering enzyme complex” means a molecular complex having a nucleic acid alteration activity and imparted with a particular nucleotide sequence recognition ability, which includes the above-mentioned complex in which a nucleic acid sequence-recognizing module, a nucleic acid altering enzyme, and a proteolysis tag are linked. The complex may be linked with a base excision repair inhibitor. The “complex” here encompasses not only one constituted of plural molecules but also one having, in a single molecule, a molecule constituting the above-mentioned complex of the present invention such as fusion proteins. Furthermore, a molecule or molecular complex that functions due to a nucleic acid sequence-recognizing module and a nucleic acid altering enzyme in integration such as restriction enzyme and CRISPR/Cas system, and is bound with a proteolysis tag is also encompassed in the complex of the present invention. In addition, “encoding a complex” encompasses both encoding respective molecules constituting the complex, and encoding a fusion protein having constituting molecules in a single molecule.

The nucleolytic enzyme used in the present invention is not particularly limited as long as it catalyzes the above-mentioned reaction, and, for example, nuclease (e.g., Cas effector protein (e.g., Cas9, Cpf1), endonuclease (e.g., restriction enzyme), exonuclease etc.), recombinase, DNA gyrase, DNA topoisomerase, transposase and the like can be mentioned.

The nucleic acid base converting enzyme to be used in the present invention is not particularly limited as long as it can catalyze the above-mentioned reaction, and examples thereof include deaminase belonging to the nucleic acid/nucleotide deaminase superfamily, which catalyzes a deamination reaction that converts an amino group to a carbonyl group. Preferable examples thereof include cytidine deaminase capable of converting cytosine or 5-methylcytosine to uracil or thymine, respectively, adenosine deaminase capable of converting adenine to hypoxanthine, guanosine deaminase capable of converting guanine to xanthine and the like. As cytidine deaminase, more preferred is activation-induced cytidine deaminase (hereinafter to be also referred to as AID) which is an enzyme that introduces a mutation into an immunoglobulin gene in the acquired immunity of vertebrata or the like.

While the derivation of nucleic acid base converting enzyme is not particularly limited, for example, PmCDA1 (Petromyzon marinus cytosine deaminase 1) derived from Petromyzon marinus, or AID (Activation-induced cytidine deaminase; AICDA) derived from mammal (e.g., human, swine, bovine, horse, monkey etc.) can be used. For example, GenBank accession Nos. EF094822 and ABO15149 can be referred to for the base sequence and amino acid sequence of cDNA of PmCDA1, GenBank accession No. NM 020661 and NP 065712 can be referred to for the base sequence and amino acid sequence of cDNA of human AID. From the aspect of enzyme activity, PmCDA1 is preferred.

The DNA glycosylase to be used in the present invention is not particularly limited as long as it can catalyze the above-mentioned reaction, and thymine DNA glycosylase, oxoguanine glycosylase, alkyladenine DNA glycosylase (e.g., yeast 3-methyladenine-DNA glycosylase (MAG1) etc.) and the like can be mentioned. The present inventor previously reported that use of a DNA glycosylase with sufficiently low reactivity with DNA having a double helix structure without distortion (unrelaxed DNA) as DNA glycosylase can reduce cytotoxicity and efficiently alter a target sequence (WO 2016/072399). Therefore, as DNA glycosylase, a DNA glycosylase with sufficiently low reactivity with DNA having a double helix structure without distortion is preferably used. Examples of such DNA glycosylase include a mutant of UNG having cytosine-DNA glycosylase (CDG) activity and/or thymine-DNA glycosylase (TDG) activity (uracil-DNA glycosylase), and UDG mutant from vaccinia virus, which are described in WO 2016/072399.

Specific examples of the aforementioned mutant of UNG include yeast UNG1 N222D/L304A double mutant, N222D/R308E double mutant, N222D/R308C double mutant, Y164A/L304A double mutant, Y164A/R308E double mutant, Y164A/R308C double mutant, Y164G/L304A double mutant, Y164G/R308E double mutant, Y164G/R308C double mutant, N222D/Y164A/L304A triple mutant, N222D/Y164A/R308E triple mutant, N222D/Y164A/R308C triple mutant, N222D/Y164G/L304A triple mutant, N222D/Y164G/R308E triple mutant, N222D/Y164G/R308C triple mutant and the like. When another UNG is used in place of the yeast UNG1, a mutant in which a similar mutation has been introduced into the amino acid corresponding to each mutant described above may be used. As UDG mutant from vaccinia virus, N120D mutant, Y70G mutant, Y70A mutant, N120D/Y70G double mutant, N120D/Y70A double mutant and the like can be mentioned. Alternatively, it may be a DNA glycosylase divided into two segments which is a split enzyme designed such that each segment is bound to either of two divided nucleic acid sequence-recognizing modules to form two complexes, the nucleic acid sequence-recognizing module can specifically bind to a target nucleotide sequence when both complexes are refolded, and the DNA glycosylase can catalyze a base excision reaction by the specific binding. The split enzyme can be designed and produced by referring to the descriptions of, for example, WO 2016/072399, Nat Biotechnol. 33 (2): 139-142 (2015), PNAS 112 (10): 2984-2989 (2015).

In the present invention, the “base excision repair” is one of the DNA repair mechanisms of living organisms, and means a mechanism for repairing damages of bases by cutting off damaged parts of the bases by enzymes and rejoining them. Excision of damaged bases is performed by DNA glycosylase, which is an enzyme that hydrolyzes the N-glycoside linkage of DNA. An abasic site (apurinic/apyrimidic (AP) site) resulting from the abasic reaction by the enzyme is treated by an enzyme at the downstream of the base excision repair (BER) pathway such as an AP endonuclease, DNA polymerase, DNA ligase and the like. Examples of such gene or protein involved in the BER pathway include, but are not limited to, UNG (NM_003362), SMUG1 (NM_014311), MBD4 (NM_003925), TDG (NM_003211), OGG1 (NM_002542), MYH (NM_012222), NTHL1 (NM_002528), MPG (NM_002434), NEIL1 (NM_024608), NEIL2 (NM_145043), NEIL3 (NM_018248), APE1 (NM_001641), APE2 (NM_014481), LIG3 (NM_013975), XRCC1 (NM_006297), ADPRT (PARP1) (NM_0016718), ADPRTL2 (PARP2) (NM_005484) and the like (parentheses indicate refseq number in which the base sequence information of each gene (cDNA) is registered).

In the present invention, the “base excision repair inhibitor” means a substance that inhibits any stage of the above-mentioned BER pathway, or a protein that eventually inhibits BER by inhibiting the expression of molecules mobilized in the BER pathway. While the base excision repair inhibitor to be used in the present invention is not particularly limited as long as it consequently inhibits BER, from the aspect of efficiency, an inhibitor of DNA glycosylase located at the upstream of the BER pathway is preferable. Examples of the inhibitor of DNA glycosylase to be used in the present invention include, but are not limited to, a thymine DNA glycosylase inhibitor, an uracil DNA glycosylase inhibitor, an oxoguanine DNA glycosylase inhibitor, an alkylguanine DNA glycosylase inhibitor and the like. For example, when cytidine deaminase is used as a nucleic acid altering enzyme, it is suitable to use a uracil DNA glycosylase inhibitor to inhibit repair of U:G or G: U mismatch of DNA generated by mutation.

Examples of such uracil DNA glycosylase inhibitor include, but are not limited to, a uracil DNA glycosylase inhibitor (Ugi) derived from Bacillus subtilis bacteriophage, PBS1, and a uracil DNA glycosylase inhibitor (Ugi) derived from Bacillus subtilis bacteriophage, PBS2 (Wang, Z., and Mosbaugh, D. W. (1988) J. Bacteriol. 170, 1082-1091). The above-mentioned inhibiter of the repair of DNA mismatch can be used in the present invention. Particularly, Ugi derived from PBS2 is also known to have an effect of making it difficult to cause mutation, cleavage and recombination other than T from C on DNA, and thus the use of Ugi derived from PBS2 is suitable.

As mentioned above, in the base excision repair (BER) mechanism, when a base is excised by DNA glycosylase, AP endonuclease puts a nick in the abasic site (AP site), and exonuclease completely excises the AP site. When the AP site is excised, DNA polymerase produces a new base by using the base of the opposing strand as a template, and DNA ligase finally seals the nick to complete the repair. Mutant AP endonuclease that has lost the enzyme activity but maintains the binding capacity to the AP site is known to competitively inhibit BER. Therefore, these mutation AP endonucleases can also be used as the base excision repair inhibitor in the present invention. While the derivation of the mutant AP endonuclease is not particularly limited, for example, AP endonucleases derived from Escherichia coli, yeast, mammal (e.g., human, mouse, swine, bovine, horse, monkey etc.) and the like can be used. For example, UniprotKB No. P27695 can be referred to for the amino acid sequence of human Apel. Examples of the mutant AP endonuclease that has lost the enzyme activity but maintains the binding capacity to the AP site include proteins having mutated activity site and mutated Mg (cofactor)-binding site. For example, E96Q, Y171A, Y171F, Y171H, D210N, D210A, N212A and the like can be mentioned for human Apel.

In the present invention, the “transcriptional regulatory factor” means a protein or a domain thereof which has an activity of promoting or suppressing target gene transcription. In the following, one having a transcription promoting activity is sometimes referred to as a “transcription activation factor”, and one having a transcription suppressing activity is sometimes referred to as a “transcription inhibitory factor”.

The transcription activation factor to be used in the present invention is not particularly limited as long as it can promote transcription of the target gene and, for example, an activated domain of HSV (Herpes simplex virus) VP16, p65 subunit of NFκB, VP64, VP160, HSF, P300 and EB virus (Epstein-Barr Virus) RTA, fusion proteins of these and the like can be mentioned. The transcription inhibitory factor to be used in the present invention is not particularly limited as long as it can suppress transcription of the target gene and, for example, KRAB, MBD2B, v-ErbA, SID (including SID concatemer (SID4X)), MBD2, MBD3, DNMT family (e.g., DNMT1, DNMT3A, DNMT3B), Rb, MeCP2, ROM2 and AtHD2A, fusion proteins of these and the like can be mentioned.

A target nucleotide sequence in a double-stranded DNA to be recognized by the nucleic acid sequence-recognizing module in the complex of the present invention is not particularly limited as long as the module specifically binds to, and may be any sequence in the double-stranded DNA. The length of the target nucleotide sequence only needs to be sufficient for specific binding of the nucleic acid sequence-recognizing module. For example, when mutation is introduced into a particular site in the genomic DNA of a mammal, it is not less than 12 nucleotides, preferably not less than 15 nucleotides, more preferably not less than 17 nucleotides, according to the genome size thereof. While the upper limit of the length is not particularly limited, it is preferably not more than 25 nucleotides.

As the nucleic acid sequence-recognizing module in the complex of the present invention, a CRISPR-Cas system wherein at least one DNA cleavage ability of the Cas effector protein is inactivated (hereinafter to be also referred to as “CRISPR-mutant Cas”), zinc finger motif, TAL effector and PPR motif and the like, as well as a fragment which contains a DNA binding domain of a protein that specifically binds to DNA, such as restriction enzyme, transcriptional regulatory factor, RNA polymerase and the like, and the like can be used, but the module is not limited thereto. When a nucleic acid altering enzyme is used, the CRISPR-Cas system in which a nucleic acid sequence-recognizing module and a nucleic acid altering enzyme are integrated (Cas effector protein of the system maintains both activities of the DNA cleavage ability) may also be used. Preferably, CRISPR-mutant Cas, zinc finger motif, TAL effector, PPR motif and the like can be mentioned.

A zinc finger motif is constituted by linkage of 3-6 different Cys2His2 type zinc finger units (1 finger recognizes about 3 bases), and can recognize a target nucleotide sequence of 9-18 bases. A zinc finger motif can be produced by a known method such as Modular assembly method (Nat Biotechnol (2002) 20: 135-141), OPEN method (Mol Cell (2008) 31: 294-301), CoDA method (Nat Methods (2011) 8: 67-69), Escherichia coli one-hybrid method (Nat Biotechnol (2008) 26:695-701) and the like. JP-B-4968498 can be referred to as for the detail of the zinc finger motif production.

A TAL effector has a module repeat structure with about 34 amino acids as a unit, and the 12th and 13th amino acid residues (called RVD) of one module determine the binding stability and base specificity. Since each module is highly independent, TAL effector specific to a target nucleotide sequence can be produced by simply connecting the module. For TAL effector, a production method utilizing an open resource (REAL method (Curr Protoc Mol Biol (2012) Chapter 12: Unit 12.15), FLASH method (Nat Biotechnol (2012) 30: 460-465), and Golden Gate method (Nucleic Acids Res (2011) 39: e82) etc.) have been established, and a TAL effector for a target nucleotide sequence can be designed comparatively conveniently. National Publication of International Patent Application No. 2013-513389 can be referred to as for the detail of the production of a TAL effector.

PPR motif is constituted such that a particular nucleotide sequence is recognized by a continuation of PPR motifs each consisting of 35 amino acids and recognizing one nucleic acid base, and recognizes a target base only by 1, 4 and ii (-2) amino acids of each motif. Motif constitution has no dependency, and is free of interference of motifs on both sides. Therefore, like TAL effector, a PPR protein specific to the target nucleotide sequence can be produced by simply connecting PPR motifs. JP-A-2013-128413 can be referred to as for the detail of the production of a PPR motif.

When a fragment of restriction enzyme, transcriptional regulatory factor, RNA polymerase and the like is used, since the DNA binding domains of these proteins are well known, a fragment which contains the domain and does not have a DNA double strand cleavage ability, can be easily designed and constructed.

When a nucleic acid altering enzyme is used, any of the above-mentioned nucleic acid sequence-recognizing modules can be provided as a fusion protein with the above-mentioned nucleic acid altering enzyme and/or a base excision repair inhibitor, or a protein binding domain such as SH3 domain, PDZ domain, GK domain, GB domain and the like and a binding partner thereof may be fused with a nucleic acid sequence-recognizing module and/or a base excision repair inhibitor, respectively, and provided as a protein complex via an interaction of the domain and a binding partner thereof. Alternatively, a nucleic acid sequence-recognizing module and/or a base excision repair inhibitor may be each fused with intein, and they can be linked by ligation after protein synthesis. The proteolysis tag may be bound with any of the constituent molecules of a nucleic acid altering enzyme complex (nucleic acid sequence-recognizing module, nucleic acid altering enzyme and base excision repair inhibitor), and may be bound with plural constituent molecules. Also, when a transcriptional regulatory factor is used, the transcriptional regulatory factor may be provided as a fusion protein with a nucleic acid sequence-recognizing module in the same manner as described above, or may be bound with a nucleic acid recognition module via the above-mentioned protein binding domain and a binding partner thereof. In the same manner as described above, the proteolysis tag may be bound as a fusion protein, or may be bound with a complex for genome editing or a constituent molecule thereof via the above-mentioned protein binding domain and a binding partner thereof. The proteolysis tag is preferably bound with the C-terminal of a complex for genome editing or a constituent molecule thereof.

In the nucleic acid of the present invention, a nucleic acid sequence-recognizing module, a proteolysis tag, a nucleic acid altering enzyme and/or a base excision repair inhibitor as necessary, or a transcriptional regulatory factor can be prepared as a nucleic acid encoding a fusion protein thereof, or in a form capable of forming a complex in a host cell after translation into a protein by utilizing a binding domain, intein and the like, or as a nucleic acid encoding each of them. The nucleic acid here may be a DNA or an RNA. When it is a DNA, it is preferably a double stranded DNA, and provided in the form of an expression vector placed under regulation of a functional promoter in a host cell. When it is an RNA, it is preferably a single strand RNA.

A DNA encoding a nucleic acid sequence-recognizing module such as zinc finger motif, TAL effector, PPR motif and the like can be obtained by any method mentioned above for each module. A DNA encoding a sequence-recognizing module of restriction enzyme, transcriptional regulatory factor, RNA polymerase and the like can be cloned by, for example, synthesizing an oligoDNA primer covering a region encoding a desired part of the protein (part containing DNA binding domain) based on the cDNA sequence information thereof, and amplifying by the RT-PCR method using, as a template, the total RNA or mRNA fraction prepared from the protein-producing cells.

A DNA encoding a nucleic acid altering enzyme and base excision repair inhibitor can also be cloned similarly by synthesizing an oligoDNA primer based on the cDNA sequence information thereof, and amplifying by the RT-PCR method using, as a template, the total RNA or mRNA fraction prepared from the enzyme-producing cells. For example, a DNA encoding PBS2-derived UGI can be cloned by designing suitable primers for the upstream and downstream of CDS based on the cDNA sequence (accession No. J04434) registered in the NCBI/GenBank database, and cloning from PBS2-derived mRNA by the RT-PCR method.

The cloned DNA may be directly, or after digestion with a restriction enzyme when desired, or after addition of a suitable linker (e.g., GS linker, GGGAR linker etc.), spacer (e.g., FLAG sequence etc.) and/or a nuclear localization signal (NLS) (each organelle transfer signal when the double-stranded DNA of interest is mitochondria or chloroplast DNA), to prepare a DNA encoding a fusion protein. In addition, a DNA encoding a fusion protein can be prepared by ligating with a DNA encoding a nucleic acid sequence-recognizing module.

A DNA encoding the complex for genome editing of the present invention can be obtained by chemically synthesizing the DNA strand, or by connecting synthesized partly overlapping oligoDNA short strands by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database disclosed in the home page of Kazusa DNA Research Institute (kazusa.or.jp/codon/index.html) can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding the complex for genome editing of the present invention can be produced, for example, by linking the DNA to the downstream of a promoter in a suitable expression vector.

As the expression vector, plasmids from Escherichia coli (e.g., pBR322, pBR325, pUC12, pUC13); plasmids from Bacillus subtilis (e.g., pUB110, pTP5, pC194); plasmids from yeast (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as λphage and the like; insect virus vectors such as baculovirus and the like (e.g., BmNPV, AcNPV); animal virus vectors such as retrovirus, vaccinia virus, adenovirus and the like, and the like are used.

As the promoter, any promoter appropriate for a host used for gene expression can be used. When a nucleolytic enzyme is used as a nucleic acid altering enzyme, since the survival rate of the host cell sometimes decreases markedly due to the toxicity, it is desirable to increase the number of cells by the start of the induction by using an inductive promoter. On the other hand, when a nucleic acid base converting enzyme and DNA glycosylase are used as a nucleic acid altering enzyme, or when a nucleic acid altering enzyme is not used, since sufficient cell proliferation can also be achieved by expressing the nucleic acid-altering enzyme complex of the present invention, a constitutive promoter can also be used without limitation.

For example, when the host is an animal cell, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. Of these, CMV promoter, SRa promoter and the like are preferable.

When the host is Escherichia coli, J23 series promoters (e.g., J23119 promoter), trp promoter, lac promoter, recA promoter, λP_{L} promoter, lpp promoter, T7 promoter and the like are preferable.

When the host is genus Bacillus, SPO1 promoter, SPO2 promoter, penP promoter and the like are preferable.

When the host is a yeast, the Gal1/10 promoter, PHO5 promoter, PGK promoter, GAP promoter, ADH promoter and the like are preferable.

When the host is an insect cell, a polyhedrin promoter, P10 promoter and the like are preferable.

When the host is a plant cell, CaMV35S promoter, CaMV19S promoter, NOS promoter and the like are preferable.

As the expression vector, besides those mentioned above, one containing an enhancer, a splicing signal, a terminator, a polyA addition signal, a selection marker such as drug resistance gene, an auxotrophic complementary gene and the like, a replication origin and the like on demand can be used.

An RNA encoding the complex of the present invention can be prepared by, for example, transcription to mRNA an in vitro transcription system known per se by using the above-mentioned expression vector containing a DNA encoding each protein as a template.

The host bacterium used for replication of the nucleic acid of the present invention is not particularly limited as long as it is a bacterium having a proteolysis system using tmRNA (ssrA). For example, genus Escherichia, genus Bacillus, genus Pseudomonas (e.g., Pseudomonas putida), genus Streptococcus (e.g., Streptococcus), genus Streptomyces, genus Staphylococcus, genus Yersinia, genus Acinetobacter, genus Klebsiella, genus Bordetella, genus Lactococcus, genus Neisseria, genus Aeromonas, gnus Francisella, genus Corynebacterium, genus Citrobacter, genus Chlamydiae, genus Haemophilus, genus Brucella, genus Mycobacterium, genus Legionella, genus Rhodococcus, genus Pseudomonas, genus Helicobacter, genus Salmonella, genus Staphylococcus, genus Vibrio, and genus Erysipelothrix and the like are used.

As the genus Escherichia, Escherichia coli K12·DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)], Escherichia coli DH5α, Escherichia coli BW25113 and the like are used.

As the genus Bacillus, Bacillus subtilis MI114 [Gene, 24, 255 (1983)], Bacillus subtilis 207-21 [Journal of Biochemistry, 95, 87 (1984)] and the like are used.

When a nucleic acid base converting enzyme or DNA glycosylase is used as a nucleic acid altering enzyme, the nucleic acid altering enzyme and/or a base excision repair inhibitor are/is provided as a complex with mutant Cas by a method similar to the coupling scheme with the above-mentioned zinc finger and the like. Alternatively, a nucleic acid base converting enzyme and/or a base excision repair inhibitor, and mutant Cas can also be bound by utilizing RNA aptamers MS2F6, PP7 and the like and RNA scaffold by binding proteins thereto.

The guide RNA forms a complementary strand with the target nucleotide sequence, mutant Cas is recruited by the tracrRNA attached and mutant Cas recognizes DNA cleavage site recognition sequence PAM (protospacer adjacent motif) (when SpCas9 is used, PAM has 3 bases of NGG (N is any base), and theoretically can target any site on the genome). One or both DNAs cannot be cleaved and, due to the action of the nucleic acid base converting enzyme or DNA glycosylase linked to the mutant Cas, nucleic acid base conversion or base excision occurs in the targeted site (appropriately adjusted within several hundred bases including whole or partial target nucleotide sequence) and a mismatch (e.g., when cytidine deaminase such as PmCDA1, AID or the like is used as the nucleic acid base converting enzyme, cytosine on the sense strand or antisense strand at the targeted site is converted to uracil to cause U:G or G: U mismatch), or apurinic/apyrimidinic site (AP site) occurs in the double stranded DNA. Various mutations are introduced due to an error in the BER system of the cell attempting to repair this. For example, the mismatch or AP site is not correctly repaired, and when repaired such that a base of the opposite strand forms a pair with a base of the converted strand (T-A or A-T in the above-mentioned example), or when other nucleotide is further substituted (e.g., U→A, G) or when one to several dozen bases are deleted or inserted during repair, various mutations are introduced. By using a base excision repair inhibitor in combination, the intracellular BER mechanism is inhibited, the frequency of repair mis increases, and the mutation introduction efficiency can be improved.

As for zinc finger motifs, production of many actually functionable zinc finger motifs is not easy, since production efficiency of a zinc finger that specifically binds to a target nucleotide sequence is not high and selection of a zinc finger having high binding specificity is complicated. While TAL effectors and PPR motifs have a high degree of freedom of target nucleic acid sequence recognition as compared to zinc finger motifs, a problem remains in the efficiency since a large protein needs to be designed and constructed every time according to the target nucleotide sequence.

In contrast, since the CRISPR-Cas system recognizes the double-stranded DNA sequence of interest with a guide RNA complementary to the target nucleotide sequence, any sequence can be targeted by simply synthesizing an oligoDNA capable of specifically hybridizing with the target nucleotide sequence.

Therefore, in a more preferable embodiment of the present invention, a CRISPR-Cas system maintaining both activities of the DNA cleavage ability, or a CRISPR-Cas system wherein DNA cleavage ability of only one or both of the Cas is inactivated (CRISPR-mutant Cas) is used as a nucleic acid sequence-recognizing module.

The nucleic acid sequence-recognizing module of the present invention using CRISPR-mutant Cas is provided as a complex of a CRISPR-RNA (crRNA) containing a sequence complementary to the target nucleotide sequence and, where necessary, trans-activating RNA (tracrRNA) necessary for recruiting mutant Cas effector protein (when tracrRNA is necessary, possibly provided as chimeric RNA with crRNA) and mutant Cas effector protein. An RNA molecule consisting of crRNA alone or a chimeric RNA of crRNA and tracrRNA that constitutes a nucleic acid sequence-recognizing module in combination with a mutant Cas effector protein is collectively referred to as a “guide RNA”. The same also applies when a CRISPR/Cas system without introduction of mutation is used.

While the Cas effector protein to be used in the present invention is not particularly limited as long as it can form a complex with guide RNA and recognize and bind to the target nucleotide sequence in the gene of interest and a protospacer adjacent motif (PAM) adjacent thereto, it is preferably Cas9 or Cpf1. Examples of Cas9 include, but are not limited to, Cas9 derived from Streptococcus pyogenes (SpCas9; PAM sequence NGG (N is A, G, T or C, hereinafter the same)), Cas9 derived from Streptococcus thermophilus (StCas9; PAM sequence NNAGAAW), Cas9 derived from Neisseria meningitidis (NmCas9; PAM sequence NNNNGATT) and the like. Preferred is SpCas9 with less restriction by PAM (substantially 2 bases, and can target theoretically any site in the genome). Examples of the Cpf1 include, but are not limited to, Cpf1 derived from Francisella novicida (FnCpf1; PAM sequence NTT), Cpf1 derived from Acidaminococcus sp. (AsCpf1; PAM sequence NTTT), Cpf1 derived from Lachnospiraceae bacterium (LbCpf1; PAM sequence NTTT) and the like. As a mutant Cas effector protein (sometimes to be abbreviated as mutant Cas) to be used in the present invention, any of Cas effector protein wherein the cleavage ability of the both strands of the double-stranded DNA is inactivated and one having nickase activity wherein at least one cleavage ability of one strand alone is inactivated can be used. For example, in the case of SpCas9, a D10A mutant in which the 10th Asp residue is converted to an Ala residue and lacking cleavage ability of a strand opposite to the strand forming a complementary strand with a guide RNA (thus having nickase activity for a strand forming complementary strand with guide RNA), or H840A mutant in which the 840th His residue is converted to an Ala residue and lacking cleavage ability of a strand forming a complementary strand to guide RNA (thus having nickase activity for a strand forming complementary strand with guide RNA, or a double mutant thereof (dCas9) can be used. In the case of FnCpf1, a mutant in which the 917th Asp residue is converted to an Ala residue (D917A) or the 1006th Glu residue is converted to an Ala residue (E1006A), and lacking cleavage ability of both strands can be used. As long as at least one of the strands of double-stranded DNA lacks cleavage ability, other mutant Cas can also be used similarly.

A DNA encoding Cas effector protein (including mutant Cas, hereinafter the same) can be cloned by a method similar to the above-mentioned method for a DNA encoding a base excision repair inhibitor, from a cell producing the enzyme. A mutant Cas can be obtained by introducing a mutation to convert an amino acid residue of the site important for the DNA cleavage activity (e.g., 10th Asp residue and 840th His residue for SpCas9, 917th Asp residue and 1006th Glu residue for FnCpf1 and the like, though not limited thereto) to other amino acids, into a DNA encoding cloned Cas, by a site specific mutation induction method known per se.

Alternatively, a DNA encoding Cas effector protein can also be constructed as a DNA with codon usage suitable for expression in a host cell to be used, by a method similar to those mentioned above for a DNA encoding a nucleic acid sequence-recognizing module and a DNA encoding DNA glycosylase, and in a combination of chemical synthesis or PCR method or Gibson Assembly method.

The obtained DNA encoding a Cas effector protein, nucleic acid altering enzyme, base excision repair inhibitor, and/or transcriptional regulatory factor can be inserted into the downstream of a promoter of an expression vector similar to the one mentioned above, according to the target cell.

On the other hand, a DNA encoding guide RNA can be obtained by designing an oligoDNA sequence linking a coding sequence of crRNA sequence containing a nucleotide sequence complementary to the target nucleotide sequence (to be also referred to as “targeting sequence” in the present specification) (e.g., when FnCpf1 is recruited as Cas effector protein, crRNA containing SEQ ID NO: 19; AAUUUCUACUGUUGUAGAU at the 5′-side of the targeting sequence can be used, and the underlined sequences form base pairs to form a stem-loop structure), or a crRNA coding sequence and, where necessary, a known tracrRNA coding sequence (e.g., as tracrRNA coding sequence when Cas is recruited as Cas9 effector protein, gttttagagctagaaatagcaagttaaaataaggctagtccgttatcaacttgaaaaagtggca ccgagtcggtgcttttttt; SEQ ID NO: 18) and chemically synthesizing using a DNA/RNA synthesizer.

The “targeted strand” here means a strand forming a hybrid with crRNA of the target nucleotide sequence, and the opposite strand, which becomes single-stranded after hybridization of the targeted strand and crRNA, is referred to as a “non-targeted strand”. Since a nucleic acid base conversion reaction is generally assumed to frequently occur on a single stranded non-targeted strand, when the target nucleotide sequence is to be expressed by one of the strands (e.g., when PAM sequence is indicated, when positional relationship of target nucleotide sequence and PAM is shown etc.), it is represented by a sequence of the non-targeted strand.

While the length of the targeting sequence is not particularly limited as long as it can specifically bind to a target nucleotide sequence, for example, it is 15-30 nucleotides, preferably 18-25 nucleotides. The selection of the target nucleotide sequence is restricted by the presence of an adjacent PAM on the 3′-side (in the case of Cas9) or 5′-side (in the case of Cpf1) of the sequence. According to the finding verified in the below-mentioned Examples, in the system of the present invention in which CRISPR-mutated Cas and cytidine deaminase are combined, the regularity exists that easily substituted C shifts toward the 5′-end as the target nucleotide sequence becomes longer. Therefore, by appropriately determining the length of the target nucleotide sequence (targeting sequence as a complementary strand thereof), the site of a base into which a mutation can be introduced can be shifted. As a result, restriction by PAM (NGG in SpCas9) can be removed at least partially, and the degree of freedom of mutation introduction is expected to be still higher.

When Cas9 is used as a Cas effector protein, a targeting sequence can be designed, for example, using a guide RNA design website open to public (CRISPR Design Tool, CRISPRdirect etc.) by listing up 20 mer sequences having PAM (e.g., NGG in the case of SpCas9) adjacent to the 3′-side from the CDS sequences of the gene of interest, and selecting a sequence that causes an amino acid change in the protein encoded by the target gene when C within 7 nucleotides from the 5′ end thereof toward 3′ direction is converted to T. An appropriate sequence can be selected even when a targeting sequence with a length other than 20 mer is used. A candidate sequence having a small number of off-target sites in the host genome of interest can be used as a targeting sequence. When the guide RNA design software to be used does not have a function to search off-target sites in the genome of the host, for example, off-target sites can be searched by applying a Blast search against the genome of the host, for example, 8-12 nucleotides on the 3′-side of the candidate sequence (seed sequence with high discrimination ability of target nucleotide sequence).

While a DNA encoding guide RNA can also be inserted into an expression vector similar to the one mentioned above. As the promoter, pol III system promoter (e.g., SNR6, SNR52, SCR1, RPR1, U3, U6, H1 promoter etc.) and terminator (e.g., polyT sequence (T6 sequence etc.)) are preferably used.

A DNA encoding guide RNA (crRNA or crRNA-tracrRNA chimera) can be obtained by designing an oligoRNA sequence linking a sequence complementary to the target strand of the target nucleotide sequence and a known tracrRNA sequence (when Cas9 is recruited) or a direct repeat sequence of crRNA (when Cpf1 is recruited) and chemically synthesizing using a DNA/RNA synthesizer.

### 2. Alteration Method of Targeted Site of Double Stranded DNA of Host Bacterium

The targeted site of the double stranded DNA of the host can be altered, or the expression of a gene encoded by the double stranded DNA in the vicinity of the targeted site can be regulated by introducing the complex and the nucleic acid of the present invention described in 1. into a host, particularly a bacterium, and culturing the host. Therefore, in another embodiment, a method for altering a targeted site of a double stranded DNA of a bacterium, including a step of bringing the nucleic acid altering enzyme complex into contact with the double stranded DNA of the host bacterium to convert one or more nucleotides in the targeted site to other one or more nucleotides or delete one or more nucleotides, or insert one or more nucleotides into said targeted site (hereinafter to be also referred to as “the alteration method of the present invention”) is provided. Using a nucleic acid base converting enzyme or DNA glycosylase as a nucleic acid altering enzyme, the targeted site can be altered without cleaving at least one of the strands of the double stranded DNA in the targeted site. In still another embodiment, a method including contacting the complex of the present invention with a double stranded DNA of the host bacterium and regulating transcription of a gene in the vicinity of the targeted site is provided.

The complex of the present invention is contacted with the double stranded DNA by introducing the complex or a nucleic acid encoding same into a bacterium having the desired double stranded DNA (e.g., genomic DNA). In consideration of the introduction and expression efficiency, it is desirable to introduce the complex for genome editing into the bacterium in the form of a nucleic acid encoding the complex, rather than as the complex itself, and express the complex in the bacterium.

Examples of the bacterium used for the alteration method of the present invention include those similar to the bacteria used for nucleic acid replication in 1.

An expression vector can be introduced by a known method (e.g., lysozyme method, competent method, PEG method, CaCl₂) coprecipitation method, electroporation method, the microinjection method, the particle gun method, lipofection method, Agrobacterium method and the like) according to the kind of the bacterium.

Escherichia coli can be transformed according to the methods described in, for example, Proc. Natl. Acad. Sci. USA, 69, 2110 (1972), Gene, 17, 107 (1982) and the like.

The genus Bacillus can be introduced into a vector according to the methods described in, for example, Molecular & General Genetics, 168, 111 (1979) and the like.

A bacterium introduced with a vector can be cultured according to a known method according to the kind of the bacterium.

For example, when Escherichia coli or genus Bacillus is cultured, a liquid medium is preferable as a medium to be used for the culture. The medium preferably contains a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is preferably about 5 to about 8.

As a medium for culturing Escherichia coli, for example, M9 medium containing glucose, casamino acid [Journal of Experiments in Molecular Genetics, 431-433, Cold Spring Harbor Laboratory, New York 1972] is preferable. Where necessary, for example, agents such as 3β-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30 to about 40° C. Where necessary, aeration and stirring may be performed.

The present inventors confirmed that when PmCDA1 is used as the nucleic acid altering enzyme, the mutation introduction efficiency is increased by culturing animal cells and plant cells at a temperature lower (e.g., 20 to 26° C., preferably, about 25° C.) than usual. When a bacterium is cultured, it is also preferable to culture at the above-mentioned low temperature.

An RNA encoding the complex of the present invention can be introduced into a host cell by microinjection method, lipofection method and the like. RNA introduction can be performed once or repeated multiple times (e.g., 2-5 times) at suitable intervals.

The present inventors also confirmed using a budding yeast that when sequence-recognizing modules are produced corresponding to the adjacent multiple target nucleotide sequences, and simultaneously used, the mutation introduction efficiency drastically increases than using a single nucleotide sequence as a target, and similar effects can also be expected in the bacterium. The effect can occur both when the nucleotide sequences are in the same direction (targeted strands are the same), and when they are opposed (both strands of double stranded DNA are targeted strands).

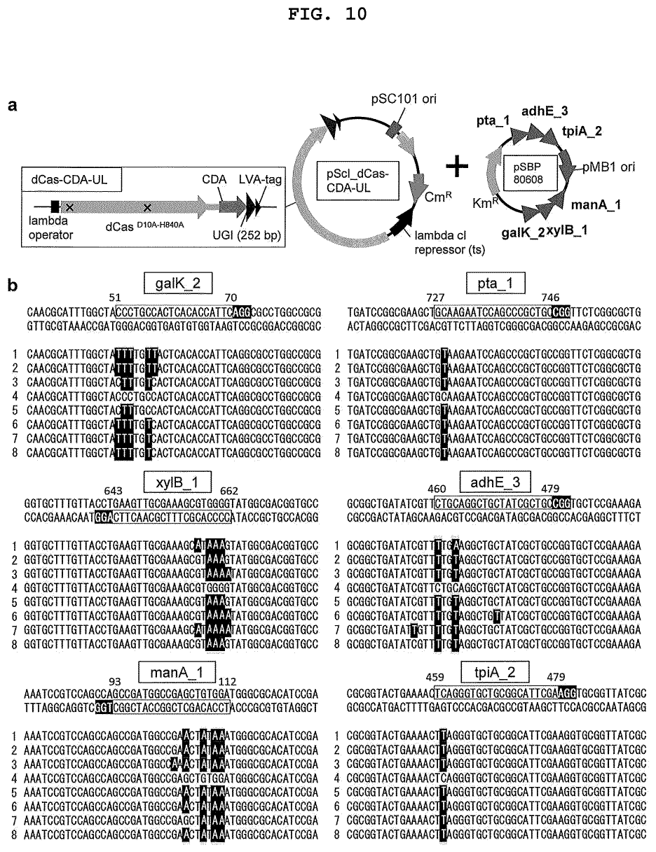

In a preferable embodiment, it was demonstrated that mutation can be simultaneously introduced into 6 sites of the genomic DNA of a bacterium by the method of the present invention (FIG. 10), and the mutation introduction efficiency is extremely high. Therefore, in the genome sequence alteration method, or target gene expression regulating method of the present invention, alteration of multiple DNA regions at completely different positions as targets, or regulation of expression of plural target genes can also be performed. Therefore, in one preferable embodiment of the present invention, two or more kinds of nucleic acid sequence-recognizing modules that specifically bind to different target nucleotide sequences (which, when target DNA is cell-endogenous DNA, may be present in one object gene, or two or more different object genes, and these object genes may be present on the same chromosome or plasmid, or different chromosome or plasmid) can be used. In this case, each one of these nucleic acid sequence-recognizing modules, and a nucleic acid altering enzyme and/or a base excision repair inhibitor, or a transcriptional regulatory factor form a complex added with a proteolysis tag. Here, a common nucleic acid base converting enzyme, a common base excision repair inhibitor and a common transcriptional regulatory factor can be used. For example, when CRISPR-Cas system is used as a nucleic acid sequence-recognizing module, a common complex (including fusion protein) of a Cas effector protein and a nucleic acid altering enzyme and/or a base excision repair inhibitor, or transcriptional regulatory factor is used, and two or more kinds of chimeric RNAs of each of two or more guide RNAs that respectively form a complementary strand with a different target nucleotide sequence, and tracrRNA are produced and used as guide RNA-tracrRNA. On the other hand, when zinc finger motif, TAL effector and the like are used as nucleic acid sequence-recognizing modules, for example, a nucleic acid altering enzyme and/or a base excision repair inhibitor, or a transcriptional regulatory factor can be fused with a nucleic acid sequence-recognizing module that specifically binds to a different target nucleotide.

To express the complex of the present invention in a host bacterium, as mentioned above, an expression vector containing a DNA encoding the complex is introduced into the host bacterium. For efficient introduction of mutation or sufficient regulation of the expression of a target gene, it is desirable to maintain an expression of the complex for genome editing at a given level or above for not less than a given period. From such viewpoint, it is certain that the expression vector is incorporated into the host genome. Since continuous expression of the complex for genome editing increases the risk of off-target cleavage, it is preferably removed immediately after achieving mutation introduction. Examples of the means for removing DNA incorporated into the host genome include a method using a Cre-loxP system, a method using transposon and the like.

Alternatively, editing of host genome can be efficiently realized while avoiding the risk of off-target cleavage by causing a nucleic acid reaction in a desired stage, and transiently expressing the complex of the present invention in a host bacterium for a period necessary for stabilizing the alteration of the targeted site. While a period necessary for the nucleic acid alteration reaction and stabilizing the alteration of the targeted site varies depending on the kind of the host bacterium, culture conditions and the like, about 2 to 3 days are considered to be necessary since at least several generations of cell division are generally necessary. Those of ordinary skill in the art can appropriately determine a preferable expression induction period based on the culture conditions and the like to be used. The expression induction period of a nucleic acid encoding the complex of the present invention may be extended beyond the above-mentioned “period necessary for stabilizing the alteration of the targeted site” as long as the host bacterium is free of side effects.

As a means for transiently expressing the complex of the present invention at a desired stage for a desired period, a method including producing a construct (expression vector) containing a DNA encoding the complex (in mutant CRISPR-Cas system, a DNA encoding a guide RNA, a DNA encoding a Cas effector protein, and, as necessary, a DNA encoding a nucleic acid altering enzyme and/or a base excision repair inhibitor, or a transcriptional regulatory factor) in a form permitting regulation of the expression period of the complex and introducing same into the host cell can be mentioned. The “form capable of regulating the expression period” is specifically, for example, a nucleic acid encoding the complex of the present invention placed under regulation of an inducible regulatory region. While the “inducible regulatory region” is not particularly limited, it is, for example, an operon of a temperature sensitive (ts) mutation repressor and an operator regulated thereby. Examples of the ts mutation repressor include, but are not limited to, ts mutation of cI repressor from λphage. In the case of λphage cI repressor (ts), it is linked to an operator to suppress expression of gene in the downstream at not more than 30° C. (e.g., 28° C.). At a high temperature of not less than 37° C. (e.g., 42° C.), it is dissociated from the operator to allow for induction of gene expression. Therefore, the period when the expression of the target gene is suppressed can be minimized by culturing a host bacterium introduced with a nucleic acid encoding the complex of the present invention generally at not more than 30° C., raising the temperature to not less than 37° C. at an appropriate stage, performing culture for a given period to carry out nucleic acid conversion reaction and, after introduction of mutation into the target gene, rapidly lowering the temperature to not more than 30° C. Thus, even when an essential gene for the host cell is targeted, it can be efficiently edited while suppressing the side effects.

When temperature sensitive mutation is utilized, for example, a temperature sensitive mutant of a protein necessary for autonomous replication of a vector is included in a vector containing a DNA encoding the complex of the present invention. As a result, autonomous replication becomes impossible rapidly after expression of the complex, and the vector naturally falls off during the cell division. Examples of the temperature sensitive mutant protein include, but are not limited to, a temperature sensitive mutant of Rep101 ori necessary for the replication of pSC101 ori. Rep101 ori (ts) acts on pSC101 ori to enable autonomous replication of plasmid at not more than 30° C. (e.g., 28° C.), but loses function at not less than 37° C. (e.g., 42° C.), and plasmid cannot replicate autonomously. Therefore, a combined use with cI repressor (ts) of the above-mentioned λphage simultaneously enables transient expression of the complex of the present invention, and removal of the plasmid.

In addition, a DNA encoding the complex of the present invention is introduced into a host bacterium under regulation of inducible promoter (e.g., lac promoter (induced by IPTG), cspA promoter (induced by cold shock), araBAD promoter (induced by arabinose) etc.), the inducing substance is added to the medium (or removed from the medium) at an appropriate stage to induce expression of the complex, culture is performed for a given period to carry out a nucleic acid alteration reaction and, introduction of mutation into the target gene, expression induction discontinuation, whereby transient expression of the complex can be realized.

The present invention is explained in the following by referring to Examples, which are not to be construed as limitative.

### EXAMPLE

In the below-mentioned Examples, experiments were performed as follows.

<Design of Strain, Plasmid, Primer and Targeted gRNA>

Escherichia coli DH5α ((F⁻ endA1 supE44 thi-1 recA1 relA1 gyrA96 deoR phoA Φ80dlacZ ΔM15 Δ (lacZYA-argF) U169, hsdR17 (rK⁻, mK⁺), λ⁻) (TaKaRa-Bio), BW25113 (lacI⁺ rrnB_{T14} ΔlacZ_{WJ16}hsdR514 ΔaraBAD_{AH33} ΔrhaBAD_{LD78} rph-1 Δ (araB-D) 567 Δ (rhaD-B) 568ΔlacZ4787 (::rrnB-3) hsdR514 rph-1) and Top10 (F-mcrA Δ (mrr-hsdRMS-mcrBC) ϕ80 lacZΔM15 ΔlacX74 nupG recA1 araD139 Δ (ara-leu) 7697 galE15 galK16 rpsL (Str^{R}) endA1 λ⁻) (Invitrogen) were used. The plasmids and primers used in the Examples are respectively shown in Table 1 and Table 2. An oligo DNA pair for constructing the targeted gRNA vector was designed as follows: 5′-tagc- (target sequence)-3′ and 5′-aaac- (reverse complementary sequence of target sequence)-3′.

TABLE 1
Plasmids used in Example
Editing target is shown in italics.

| Plasmids | Characteristics | Reference |
|---|---|---|
| General plasmids | | |
| pSC101 | Tc^{r}, repA 101(Ts), | Lab stock |
| pWY121 | Amp^{r}, Erm^{r}, B. subtilis recombination vector | Wang, 2012 (23) |
| | encoding λ cI857, λ β, λ % exo and cre recombinase genes | |
| pTAKN2 | Kan^{r}, pMB1 ori, cloning vector | Lab stock |
| Cas/CRISPR plasmids | | |
| pCas9 | Cm^{r}, p15A ori, cas9, tracrRNA, repeat-BsaI_spacer-repeat | Jiang, 2013 (13) |
| pCRISPR | Kan^{r}, repeat-BsaI_spacer-repeat | Jiang, 2013 (13) |
| pScI_dCas9-PmCDA1_{—} | Cm^{r}, repA 101(Ts), λ cI857.dCas9-PmCDA1, | This study |
| J23119-sgRNA | J23119-sgRNA-unit | |
| pSBP307 | pScI_dCas9-PmCDA1_J23119-galK_{—}9 | This study |
| pSBP304 | pScI_dCas9-PmCDA1_J23119-rpoB_{—}1 | This study |
| pScI | Cm^{r}, repA 101(Ts), λ cI857, | This study |
| pScI_dCas | Cm^{r}, repA 101(Ts), λ cI857, dCas9 | This study |
| pScI_dCas9-PmCDA1 | Cm^{r}, repA 101(Ts), λ c1857, dCas9-PmCDA1 | This study |
| pScI_dCas9-PmCDA1-LVA | Cm^{r}, repA 101(Ts), λ cI857, dCas9-PmCDA1-LVA | This study |
| pScI_dCas9-PmCDA1-UGI-LVA | Cm^{r}, repA 101(Ts), λ c1857, dCas9-PmCDA1-UGI-LVA | This study |
| pTAKN2_J23119-sgRNA | Kan^{r}, pTAKN2, J23119-sgRNA-units | This study |
| pSBP804 | pTAKN2_J23119-galK_{—}10-galK_{—}11-galK_{—}13 | This study |
| pSBP806 | pTAKN2_J23119-galK_{—}2-xylB_{—}1-manA_{—}1 | This study |
| pSBP808 | pTAKN2_J23119-pta_{—}1-adhE_{—}3-tpiA_{—}2 | This study |
| pSBP80608 | pTAKN2_J23119-galK_{—}2-xylB_{—}1-manA_{—}1-pta_{—}1adhE_{—}3-tptA_{—}2 | This study |
| pTAKN-IS1235 | pTAKN2_J23119-IS1-IS2-IS3-IS5 | This study |
| pSBP8001 | Kan^{r}, pTAKN2_J23119-galK_{—}1 | This study |
| pSBP8003 | pTAKN2_J23119-galK_{—}2 | This study |
| pSBP8004 | pTAKN2_J23119-galK_{—}3 | This study |
| pSBP8005 | pTAKN2_J23119-galk_{—}4 | This study |
| pSBP8006 | pTAKN2_J23119-galK_{—}5 | This study |
| pSBP8007 | pTAKN2_J23119-galK_{—}6 | This study |
| pSBP8008 | pTAKN2_J23119-galK_{—}7 | This study |
| pSBP8009 | pTAKN2_J23119-galK_{—}8 [20 nt] | This study |
| pSBP8010 | pTAKN2_J23119-galk_{—}8 [21 nt] | This study |
| pSBP8011 | pTAKN2_J23119-galK_{—}9 [20 nt] | This study |
| pSBP8012 | pTAKN2_J23119-galk_{—}9 [21 nt] | This study |
| pSBP8013 | pTAKN2_J23119-galK_{—}10 | This study |
| pSBP8014 | pTAKN2_J23119-galK_{—}11 [20 nt] | This study |
| pSBP8015 | pTAKN2_J23119-galK_{—}11 [23 nt] | This study |
| pSBP8016 | pTAKN2_J23119-galK_{—}12 | This study |
| pSBP8017 | pTAKN2_J23119-galk_{—}13 [20 nt] | This study |
| pSBP8018 | pTAKN2_J23119-galK_{—}13 [21 nt] | This study |
| pSBP8019 | pTAKN2_J23119-galK_{—}14 | This study |
| pSBP8025 | pTAKN2_J23119-gsiA [A18 nt] | This study |
| pSBP8021 | pTAKN2_J23119-gsiA [A20 nt] | This study |
| pSBP8022 | pTAKN2_J23119-gsiA [A22 nt] | This study |
| pSBP8023 | pTAKN2_J23119-gsiA [A24 nt] | This study |
| pSBP8029 | pTAKN2_J23119-gsiA [B18 nt] | This study |
| pSBP8027 | pTAKN2_J23119-gsiA [B20 nt] | This study |
| pSBP8040 | pTAKN2_J23119-gsiA [B22 nt] | This study |
| pSBP8042 | pTAKN2_J23119-gsiA [B24 nt] | This study |
| pSBP8043 | pTAKN2_J23119-gsiA [C18 nt] | This study |
| pSBP8030 | pTAKN2_J23119-gsiA [C20 nt] | This study |
| pSBP8045 | pTAKN2_J23119-gsiA [C22 nt] | This study |
| pSBP8047 | pTAKN2_J23119-gsiA [C24 nt] | This study |
| pSBP8048 | pTAKN2_J23119-ycbF [18 nt] | This study |
| pSBP8050 | pTAKN2_J23119-ycbF [20 nt] | This study |
| pSBP8052 | pTAKN2_J23119-ycbF [22 nt] | This study |
| pSBP8037 | pTAKN2_J23119-yfiH [18 nt] | This study |
| pSBP8035 | pTAKN2_J23119-yfiH [20 nt] | This study |
| pSBP8056 | pTAKN2_J23119-yfiH [22 nt] | This study |

TABLE 2-1
Primers used in Example

| | | SEQ ID | |
|---|---|---|---|
| Name | Sequence (5′ -> 3′) | NO | Purpose |
| p346 | GCTACTCTAGAAAAAAAAACCCCGC | 20 | Xba1-rhoTer-chmrUnit R |
| | CCTGTCAGGGGCGGGGTTTTTTTTT | | |
| | CGGGGTTTAGCAAGATGGCAGCGC | | |
| p426 | GCCTGATCGATGCATCAGAAAATTA | 21 | Cla1-upE-P3-chmrUnit F2 |
| | TTTTAAATTTCCTCTTGACAGCTAG | | |
| | CTCAGTCCTAGGTATAATGCTAGCA | | |
| | GAGACCCGGGATGGTC | | |
| p597 | GATCCTTTTTGATAATCTCGTCGAC | 22 | Sal1-gRNArepeat_F |
| | ATAACAATTGAGCAAGAATCTTCAT | | |
| | CG | | |
| p598 | CTCACGTTAAGGGATTTIGGTCATC | 23 | Pst1 gRNArepeat R |
| | TGCAGTGTATGCGTATTTGCGCGCT | | |
| | G | | |
| p599 | GAAGATTCTTGCTCAATTGTTATGT | 24 | Sal1-pTAKN2-Kan-down |
| | CGACGAGATTATCAAAAAGGATCTT | | |
| | CACCTAG | | |
| p600 | CAGCGCGCAAATACGCATACACTGC | 25 | Pst1-pTAKN2-colE1-up |
| | AGATGACCAAAATCCCTTAACGTGA | | |
| | G | | |
| p669 | CTGCACGCGCACTTTTATCC | 26 | galK_up150_F |
| p260 | CTCTGTTTGCCAACGCATTTG | 27 | galK 50 F |
| p259 | CAATGGTGACATCACGCAGG | 28 | galK 600 R |
| p141 | CCTGGGCGATAACGTAGTIGC | 29 | rpoB 2K R |
| p143 | CCTCGGCAACCGTCGTATCC | 30 | rpoB 1.5K F |
| p490 | CCGGTTATCGGTAGCGATAC | 31 | xy1B up F |
| p491 | GCCTGGGGATTATTGTGTGG | 32 | xy1B_1K_R |
| p470 | GCGGCTCCAGGTTACTTCC | 33 | manA_up50_F |
| p471 | GCCATCACTTCCAGCGC | 34 | manA_0.9K_R |
| p496 | GCCAAATCGGCGGTAACG | 35 | pta_up_F |
| p497 | CCATTTCGTAACCGCCAGTC | 36 | pta_0.9K_R |
| p502 | CTCTCGTATTCGAGCAGATG | 37 | adhE_up_F |
| p503 | CCGCCCATAGCAACCAG | 38 | adhE_1K_R |
| p508 | GGGGCGGCCATCTTCC | 39 | tpiA_up_F |
| p509 | CAGCCGAGGAAATTCAGG | 40 | tpiA_down_R |
| p685 | TCTTTCCCTACACGACGCTCTTCCG | 41 | NGSadaptor-galK_F1 |
| | ATCTCCGCAGAACAGGCAGCAGAG | | |
| p686 | GTGACTGGAGTTCAGACGTGTGCTC | 42 | NGSadaptor-galK_R1 |
| | TTCCGATCTGACAATGGGCGCATCG | | |
| | AGGG | | |
| p687 | TCTTTCCCTACACGACGCTCTTCCG | 43 | NGSadaptor-galK_F2 |
| | ATCTCACACCGACTACAACGACGG | | |
| p688 | GTGACTGGAGTTCAGACGTGTGCTC | 44 | NGSadaptor-galK_R2 |
| | TTCCGATCTGCTGCTGCAATACGGT | | |
| | TCCG | | |
| p689 | TCTTTCCCTACACGACGCTCTTCCG | 45 | NGSadaptor-galK_F3 |
| | ATCTCTTCGGCGGCGTGGACATGG | | |
| p690 | GTGACTGGAGTTCAGACGTGTGCTC | 46 | NGSadaptor-galK R3 |
| | TTCCGATCTACGACAGCCACACCTT | | |
| | IGGGC | | |
| p691 | TCTTTCCCTACACGACGCTCTTCCG | 47 | NGSadaptor-gisA_F |
| | ATCTGGCGGTGGAAACGGGTACCGT | | |
| | CG | | |
| p692 | GTGACTGGAGTTCAGACGTGTGCTC | 48 | NGSadaptor-gsiA_R |
| | TTCCGATCTCGCCCACCAGCGATAG | | |
| | CGTT | | |
| p693 | TCTTTCCCTACACGACGCTCTTCCG | 49 | NGSadaptor-ycbF_F |
| | ATCTGGCGCTCTTGATTTTCGCCG | | |
| p694 | GTGACTGGAGTTCAGACGTGTGCTC | 50 | NGSadaptor-ycbF_R |
| | TTCCGATCTGGATGGCACGCTGGCA | | |
| | ATGC | | |
| p695 | TCTTTCCCTACACGACGCTCTTCCG | 51 | NGSadaptor-yfiH_F |
| | ATCTGCCTCCCTGTGCTGTTTTGC | | |
| p696 | GTGACTGGAGTTCAGACGTGTGCTC | 52 | NGSadaptor-yfiH R |
| | TTCCGATCTGCTCAACACCCACGTT | | |
| | CGCC | | |

TABLE 2-2

| | | SEQ | | | | SEQ | |
|---|---|---|---|---|---|---|---|
| Name | Sequence (5' -> 3') | ID NO | Purpose | Name | Sequence (5' -> 3') | ID NO | Purpose |
| 1631 | GCTCATTATTTGCCCGCTTG | 53 | IS1-1 F | 716 | CGGAGGAAACAGAATCAGTGTG | 97 | IS2-11 F |
| 1632 | TGCCGGTTGCCAGATAGTC | 54 | IS1-1 R | 722 | GAGATGGTGGAGATCCTCTCG | 98 | IS2-11 R |
| 1683 | GGTCTTCAGGAAATCACCG | 55 | IS1-2 F | 717 | GATAGTTAGCGATGCCGGG | 99 | IS2-12 F |
| 1684 | CGTTCAACCACTTCAGTGTC | 56 | IS1-2_R | 723 | GGAGAATCCCCAGGTTATCTGG | 100 | IS2-12 R |
| 1639 | CACAAAGCTGTAAATCAGCG | 57 | IS1-3 F | 718 | TGAAACGTGCGGGTCTCAAC | 101 | IS2-13 F |
| 1640 | GTCAATGCAACACCCCTTTC | 58 | IS1-3_R | 724 | GGATAGTGGTTAATGGTGGCGTC | 102 | IS2-13 R |
| 1635 | CTACAACCAGGTCGAGTCAG | 59 | IS1-4 F | 1641 | CGTGCTGAGGGCTATTTACC | 103 | IS3-1 F |
| 1636 | GTAATCCTGCACCTCCATCAC | 60 | IS1-4 R | 1642 | GACGTCATCATTTAGCCAGATG | 104 | IS3-1 R |
| 1628 | GCCAGTAGTACCCGTCGTTG | 61 | IS1-5 F | 1643 | GGTTCTCAGGTTAATGTTTCGG | 105 | IS3-2 F |
| 1627 | CACAAGTCGTATTTCCAGAGG | 62 | IS1-5_R | 1644 | CACCAGATACTACGTTACCG | 106 | IS3-2 R |
| 1637 | CTGCAATAAGCAGAACCACC | 63 | IS1-6 F | 1645 | TTCGGACTGAAAGGAGCAAG | 107 | IS3-3 F |
| 1638 | TGTTGTGCGGTAAGTGTCTG | 64 | IS1-6_R | 1646 | AGATTCGTGCTCACCTTTCC | 108 | IS3-3 R |
| 1629 | GAGCAATGGATGGATTCGAAG | 65 | IS1-7 F | 1647 | GATTAGTATTGGCGCTGTTGTG | 109 | IS3-4 F |
| 1630 | TGAACAACTGTCCATGATTTCG | 66 | IS1-7 R | 1648 | CAGTCCATTTCACCGTATGAG | 110 | IS3-4 R |
| 703 | GGTACTTTCCGGGCAACCG | 67 | IS1-8 F | 1649 | ACAGACGACCAGAGTAATGTC | 111 | IS3-5 F |
| 708 | CTGCCATTAGCGCAGCCA | 68 | IS1-8 R | 1650 | TGGTTACGCGCTTTCATGG | 112 | IS3-5 R |
| 704 | CATAGCTCTACACGCCAGG | 69 | IS1-9 F | 1651 | CAGGCTGAACATGGATAAGAC | 113 | IS5-1 F |
| 709 | ATCATGGGCTCCTTTTAGTTGC | 70 | IS1-9 R | 1652 | ACGTATGGACATCTAAACATCC | 114 | IS5-1 R |
| 705 | GATATTGCCCGCCGGACAC | 71 | IS1-10 F | 1653 | GCAAGGTTGTGCTTCTAAAGG | 115 | IS5-2 F |
| 710 | CGATCTAAAGCGCGCAGC | 72 | IS1-10_R | 1654 | CCTGCAATCTAAAGGTAAGGATC | 116 | IS5-2 R |
| 706 | GACGTTGTTGAAAATGTAGGGT | 73 | IS1-11 F | 1655 | GATTGCTGTGGCAGGTTTAC | 117 | IS5-3 F |
| 711 | GCCTAACGCCTTTAATTCAGG | 74 | IS1-11 R | 1656 | CAGTACAACCTAGTTGCACC | 118 | IS5-3 R |
| 707 | TGTTGTGGAGCCTGAACGG | 75 | IS1-12 F | 1657 | TGAAGATTCCGTGCGTAACC | 119 | IS5-4 F |
| 712 | GCAACTGTTCCGGCAGATG | 76 | IS1-12 R | 1658 | CACGATGAAACCGTCAGTG | 120 | IS5-4_R |
| 1556 | CGGATTAATGATAAGTGGATCAG | 77 | IS2-1 F | 1659 | ATGCTACTGCCGGAACAAC | 121 | IS5-5 F |
| 1563 | CTTAGTGAATATTTGCCGACG | 78 | IS2-1 R | 1660 | TGATGTCAGCGAGAAGATGG | 122 | IS5-5 R |
| 1557 | GCTGATAAGTTACCTCCTGACC | 79 | IS2-2 F | 1661 | AGCACAGGTCAATATCTTCAC | 123 | IS5-6 F |
| 1564 | GCGACTATACAGGTTATTGACC | 80 | IS2-2 R | 1662 | AATATAGACCCGCAGATGATG | 124 | IS5-6 R |
| 1685 | ACATTACAGAGAAGCCGATG | 81 | IS2-3_F | 1663 | TCCGCCAGGATTGATTTTCG | 125 | IS5-7 F |
| 1686 | GTGATAGTTAGCGATGCCG | 82 | IS2-3 R | 1664 | CTCCGGGTATGGAGCTATG | 126 | IS5-7 R |
| 1559 | GGACGAATAAACGCATAATTAC | 83 | IS2-4 F | 1665 | GATCAGGACGCTCATATTCG | 127 | IS5-8 F |
| 1566 | TCCCAACCTTCTGTCACAG | 84 | IS2-4 R | 1666 | CTGTCATGTCGGTTAGTTCC | 128 | IS5-8 R |
| 1560 | CAATTTTCGCACCGGAATC | 85 | IS2-5 F | 1667 | ACAGGATGAAAGTCTTTGCC | 129 | IS5-9 F |
| 1567 | ATGGAGATACGACAATCAGC | 86 | IS2-5 R | 1668 | GCAATTTCCGCTTTTGCTCG | 130 | IS5-9_R |
| 1561 | CAATTCCTGGAACATTATCCG | 87 | IS2-6 F | 1681 | AACTGCTTCTCCTCACCATC | 131 | IS5-10_F |
| 1568 | TGAGTGATGTTTTGGCGAC | 88 | IS2-6 R | 1682 | AGAATCGTCTGGCGGTTG | 132 | IS5-10 R |
| 1562 | CTGTACTCACAGGGTGATG | 89 | IS2-7 F | 1671 | CATCAGAATCAATGCTGCG | 133 | IS5-11 F |
| 1569 | GGCAGACAGTTTGAAACC | 90 | IS2-7 R | 1672 | TCGCTGACTTCAGTTTCGC | 134 | IS5-11_R |
| 713 | CGCCACGAACGTAGTTAGC | 91 | IS2-8 F | 1673 | GCCTGCCAGATGATATGGTC | 135 | IS5-12 F |
| 719 | GATTGGTGAACACACCGACTAC | 92 | IS2-8 R | 1674 | ACCAGACCGTGGTTGTTAG | 136 | IS5-12 R |
| 714 | GGTCAGGTGGTTTGGAAAGC | 93 | IS2-9 F | 725 | TTTGTTATCCAGCCATGATGTTTTC | 137 | IS5-13 F |
| 720 | AAGTGGACACGCTATACCTGC | 94 | IS2-9 R | 728 | TTCCTGTATACCTGAAACGACAATG | 138 | IS5-13 R |
| 715 | CACTCAACACATACCGTGCC | 95 | IS2-10 F | 726 | CACGCACATACAACGGAGGG | 139 | IS5-14 F |
| 721 | CAACACCAAACTGGAACACGG | 96 | IS2-10 R | 729 | TTGACTGTGCGCAACATCCC | 140 | IS5-14 R |
| | | | | 727 | CCTATTCCGCCCATGACC | 141 | IS5-15 F |
| | | | | 730 | CAAAGGTCCAGGCTTTTGGG | 142 | IS5-15 R |

### <Plasmid Construction>

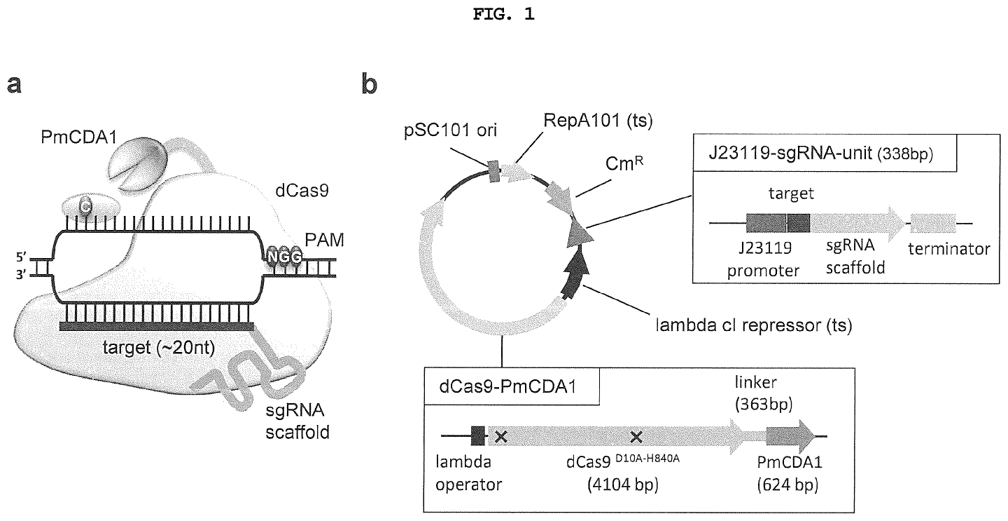

pCas9 and pCRISPR plasmids were obtained from the Marraffini lab (non-patent literature 8) via Addgene. Nickase Cas9:nCas9 (D10A or H840A) and nuclease lack Cas9: dCas9 (D10A and H840A) (SEQ ID NO: 1 and 2) (Jinek, M. et al., Science 337, 816-822 (2012).) were produced by the PCR method. PmCDA1 (SEQ ID NO: 3 and 4) was fused to the C-terminus of nCas9 or dCas9 with 121 amino acid peptide linkers (SEQ ID NO: 5 and 6) (FIG. 1).

A plasmid pScI_dCas9-PmCDA1 J23119-sgRNA carries sgRNA unit (SEQ ID NO: 15) driven by synthetic constitutive promoter J23119 (BBa_J23119 in the registry for standard biological parts) (parts.igem.org/Part: BBa_J23119) (SEQ ID NO: 16) amplified by PCR using p346/p426. sgRNA-expression unit contains two BsaI restriction sites for insertion target sequence. A pair of oligo DNA that contains target sgRNA sequence was annealed and ligated into BsaI-digested pScI_dCas9-PmCDA1_sgRNA.

pScI and pScI_dCas9 are carrying only A operator and operator-dCas9, respectively. pScI_dCas9-PmCDA1 is carrying dCas9-PmCDA1 gene. The degradation tag (LVA tag) and UGI gene are added to the C-terminus of the dCas9-PmCDA1 gene, resulting plasmid pScI_dCas9-PmCDA1-LVA and pScI dCas9-PmCDA1-UGI-LVA, respectively.

The vector plasmid pTAKN-2 has pMB1 origin compatible with pSC101. sgRNA-unit with the promoter J23119 was digested from synthetic oligonucleotide by EcoRI-HindIII, followed by ligation to the cloning vector pTAKN2. The plasmid harboring tandem three target sequences (pSBP804, galK_10-galK_11-gal_13; pSBP806, galK_2-xylB_1-manA_1; pSBP808, pta_1-adhE_3-tpiA_2) were constructed using Golden Gate assembly of the PCR product using BsaI digestion-ligation (Engler, C. et al., PLOS One 4, (2009).). The plasmid harboring different six target sequences (pSBP80608) was constructed using Gibson Assembly of the PCR products amplified from pSBP808 (pta_1-adhE_3-tpiA_2 tandem-sequence) by primers p597/598 and from pSBP806 (vector and galK_2-xylB_1-manA_1 tandem-sequence) by primers p599/p600. For IS-editing plasmid, sgRNA-expressing units were tandemly aligned in the order of IS1, IS2, IS3 and IS5.

### <Mutagenesis Assay>

DH5α or BW25113 cells chemically transformed with objective plasmid (s) were pre-cultured with 1 mL SOC medium (2% Bacto Tryptone, 0.5% Yeast Extract, 10 mM NaCl, 2.5 mM KCl, 1 mM MgSO₄ and 20 mM glucose). After incubation for 2-3 hr at 28° C., cell cultures were diluted 1:10 into 1 ml of Luria-Bertani (LB) broth or terrific broth (TB), supplemented with the following antibiotics as needed: chloramphenicol (25 μg/ml) and/or kanamycin (30 μg/ml), and grown overnight at 28° C. in 100K rpm using maximizer (TAITEC, Saitama, Japan). Next day, cell cultures were diluted 1:10 into 1 ml media again and cultured for 6 hr at 37° C. for induction, followed by overnight incubation at 28° C. The cell cultures were then spotted in serial dilution onto LB or TB agar plates supplemented with appropriate antibiotics, incubated overnight at 28° C. to form single colonies.

For positive-selection for galK gene disruption, cells were grown in M63 minimal medium (2 g/L (NH₄)₂SO₄, 13.6 g/L KH₂PO₄, 0.5 mg/L FeSO₄-7H₂O, 1 mM MgSO₄, 0.1 mM CaCl₂) and 10 μg/ml thiamine) containing 0.2% glycerol and 0.2% 2-deoxy-galctose (2-DOG) (Warming, S. et al., Nucleic Acids Res. 33, 1-12 (2005).). For selection for rifampicin-resistant mutation of rpoB gene, cells were grown in LB broth containing 50 μg/ml rifampicin. For the sequencing analysis, colonies were randomly collected and directly amplified using appropriate primers by PCR and were analyzed by Sanger sequencing using a 3130XL Genetic Analyzer (Applied Biosystems). The t-test statistical analysis was done using Excel software (Microsoft).

### <Whole Genome Sequencing>

BW25113 cells harboring each expression construct (dCas9, dCas9-PmCDA1, dCas9-PmCDA1-LVA-UGI and dCas9-PmCDA1 with rpoB_1 target) were pre-incubated overnight, diluted 1:10 into 1 mL LB media and grown for 6 hr at 37° C. for induction, followed by overnight incubation at 28° C. Cells were spread onto rifampicin-containing plate medium to isolate single colonies. Each three independent colonies were inoculated into TB medium. Genomic DNA was extracted using Wizard Genomic DNA Purification Kit (Promega) and then fragmented by sonication using Bioruptor UCD-200 TS Sonication System (Diagenote) to obtain fragments with size distribution at 500-1000 bp. Genomic DNA library was prepared by using NEBNext Ultra DNA Library Prep Kit for Illumina (New England Biolabs) and labeled by Dual Index Primers. Size selection of the library was done using Agencourt AMPure XP (Beckman Coulter) to obtain tagged fragments with length ranging from 600 to 800 bp. Size distribution was evaluated by the Agilent 2100 Bioanalyzer system (Agilent Technologies). DNA was quantified using Qibit HS dsDNA HS Assay Kit and fluorometer (Thermo Fisher Scientific). Sequencing was performed using MiSeq sequencing system (Illumina) and Miseq Reagent Kit v3 to obtain 2×300 bp read length, expecting approximately 20-fold coverage for the genome size. Data analysis was done by using CLC Genomic Workbench 9.0. (CLC bio). The sequence reads were paired and overlapping reads within a read pair were merged then trimmed based on a quality limit of 0.01 with a maximum of 2 ambiguities. Reads were mapped to Escherichia coli BW25113 reference genome by the following setting (Masking mode=no masking, Mismatch cost=2, Insertion cost=3, Deletion cost=3, Length fraction=0.5, Similarity fraction=0.8, Global alignment=No, Auto-detect paired distances=Yes, Nonspecific match handling=ignore). Local realignment was done with defaults settings (Realign unaligned ends=Yes, Multi-pass realignment=2). The variant calling was performed with the following settings (Ignore positions with coverage=1,000,000, Ignore broken pairs=Yes, Ignore Nonspecific matches=Reads, Minimum coverage=5, Minimum count=2, Minimum frequency=50%, Base quality filter=No, Read detection filter=No, Relative read direction filter=Yes, Significance=1%, Read position filter=No, Remove pyro-error variants=No). Output file was arranged using Excel (Microsoft).

### <Deep Sequencing of Target Regions>

DH5α cells expressed dCas9-PmCDA1 or dCas9-PmCDA1-UGI-LVA with gRNA targeting galK, gsiA, ycbF or yfiH genes were incubated overnight, diluted 1:10 into 1 mL LB media and grown for 6 hr at 37° C. for induction. Cell cultures were collected and genomic DNA was extracted. Target region-containing fragment (˜0.3 kb) was directly amplified using primer pairs (p685-p696) from the extracted genomic DNA. The amplicon was labeled by Dual Index Primers. More than 30,000 reads per sample on average were analyzed by MiSeq sequencing system. The sequence reads were paired and trimmed based on a quality limit of 0.01 with a maximum of 2 ambiguities and then overlapping reads within a read pair were merged. Reads were mapped to each reference sequence by the following settings (Masking mode=no masking, Mismatch cost=2, Insertion cost=3, Deletion cost=3, Length fraction=0.5, Similarity fraction=0.8, Global alignment=No, Auto-detect paired distances=Yes, Nonspecific match handling=Map randomly). Output file was arranged using Excel.

### Example 1 Deaminase-Mediated Target Mutagenesis in Escherichia Coli

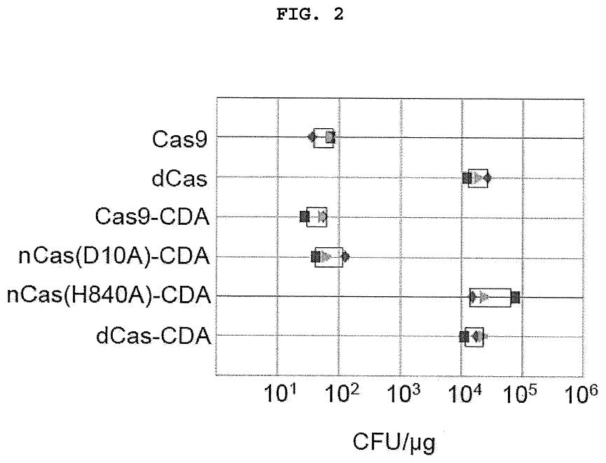

To assess whether the deaminase-mediated targeted mutagenesis is applicable in bacteria, a bacterial Target-AID vector was constructed that expresses catalytically inactivated Cas9 (dCas: D10A and H840A mutations) fused to the cytosine deaminase PmCDA1 from P. marinus (sea lamprey) (non-patent literature 11) (CDA) under temperature-inducible λ operator system (Wang, Y. et al., Nucleic Acids Res. 40, (2012).) and 20-nucleotides (nt) target sequence-gRNA scaffold hybrid (sgRNA) under a synthetic constitutive promoter J23119 (FIG. 1b). While nickase Cas9 (nCas: a D10A mutation) in combination with the deaminase can be used in eukaryotes to perform higher mutational efficiency (non-patent literatures 10, 11), the plasmid expressing nCas (D10A)-CDA showed poor transformation efficiency, suggesting that it causes severe cell growth and/or cell death in Escherichia coli, similarly to full Cas9 nuclease (FIG. 2). On the other hand, nCas (H840)-CDA showed transformation efficiency similarly as high as dCas and dCas-CDA, and was advantageous for cell proliferation and cell survival.

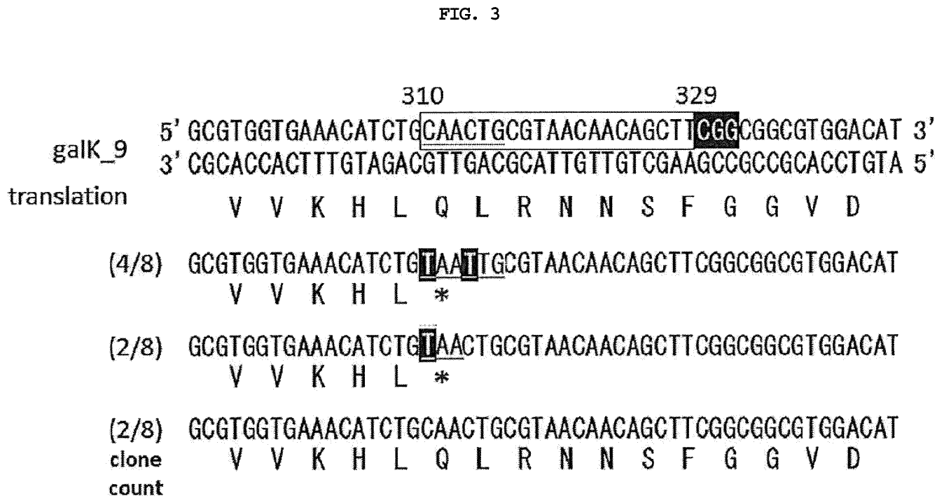

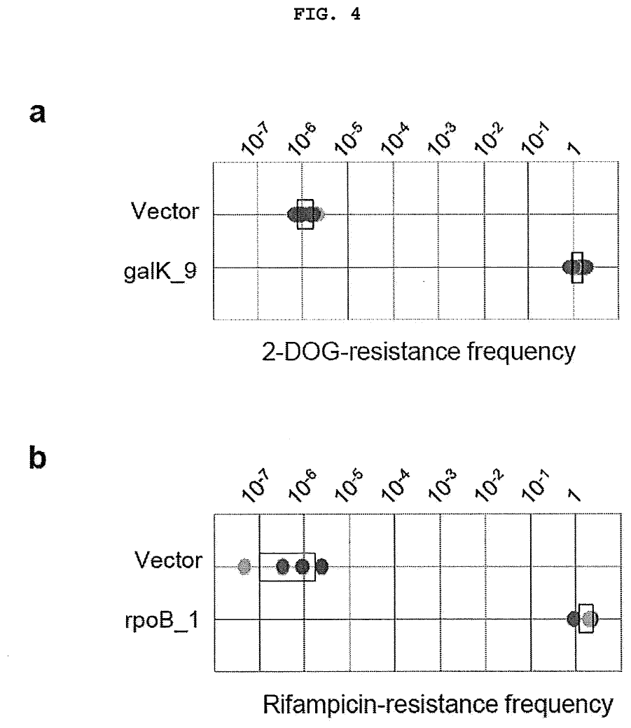

To assess the efficacy of targeted mutagenesis quantitatively, galK gene was used as a target whose loss of function can be positively selected by a galactose analog 2-deoxi-D-galactose (2-DOG), which is catalyzed by the galK gene product galactokinase to form a toxic compound (Warming, S. et al., Nucleic Acids Res. 33, 1-12 (2005).). As Target-AID is known to induce mutations at cytosine nucleotides (C) located around 15-20 bases with core region of 16-19 bases upstream of the protospacer adjacent motif (PAM) sequence (non-patent literature 11) (FIG. 1(a)). Targeting sequence was selected in the galK gene to introduce a stop codon (FIG. 3) and induced nearly 100% viability against 2-DOG, suggesting highly efficient mutagenesis (FIG. 4(a)). Six out of eight colonies were mutated as expected when sequencing analysis was performed on the cells from non-2-DOG selective medium. C to T substitutions were observed at −17 and/or −20 positions as expected.

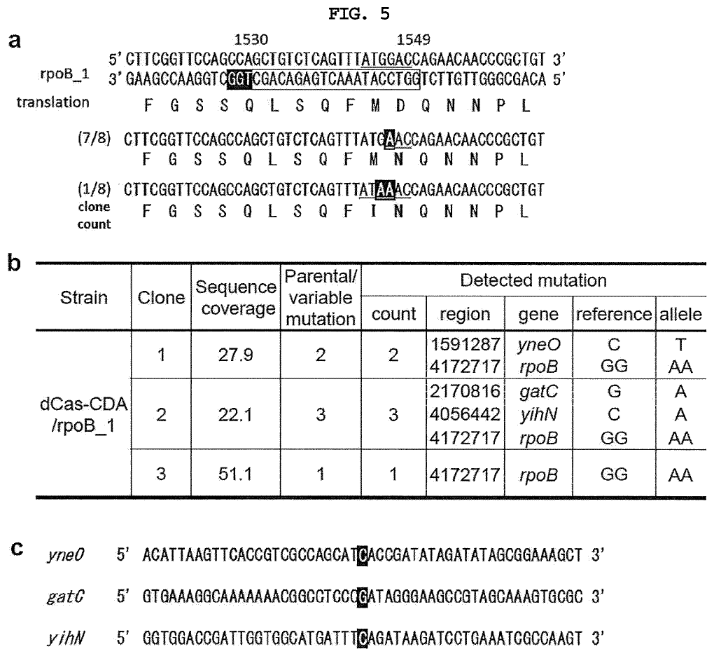

Next, an essential gene rpoB was targeted that encodes the β-subunit of RNA polymerase. While disturbing gene function of rpoB would cause cell growth suppression or cell death, specific point mutations in rpoB gene are known to confer rifampicin resistance (Jin, D. J. et al., J. Mol. Biol. 202, 245-253 (1988).). A targeting sequence was designed to induce a point mutation that confers rifampicin resistance (FIG. 5(a)). With no obvious growth suppression, transformed cells gained rifampicin resistance in nearly 100% frequency (FIG. 4(b)). Sequencing analysis of the non-rifampicin selected clones confirmed the C to T substitutions at −16 and/or −17 positions from the PAM sequence (position 1545 and 1546 of the rpoB gene) as expected (FIG. 5(a)). Whole genome sequencing was performed to assess the possible non-specific mutagenesis effect of the Target-AID in Escherichia coli. Three independent clones expressing dCas-CDA and the rpoB_1 targeting sgRNA were analyzed and found to contain zero to two unique single nucleotide variants (SNVs) at apparently unrelated genomic position (FIG. 5(b)). Adjacent sequences of the detected SNVs did not show any similarity to the rpoB target sequence (FIG. 5(c)).

### Example 2 Effect of sgRNA Length and Uracil DNA Glycosylase Inhibitor on the Mutational Frequency and Positions

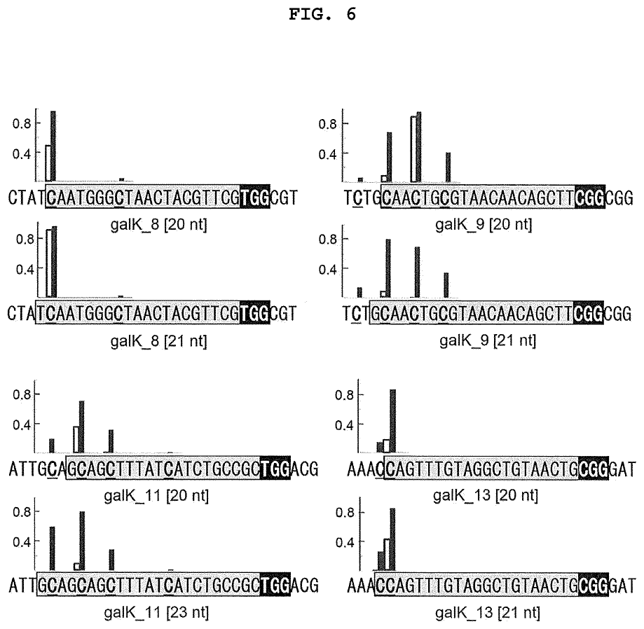

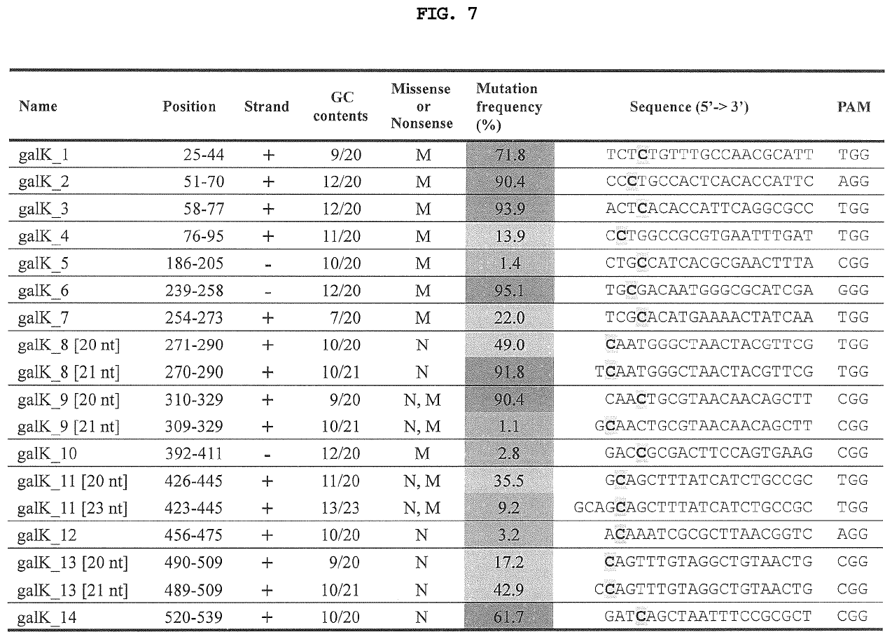

To comprehensively analyze mutational efficiency and position, deep sequencing analysis was performed using 18 targeting sequences at galK gene (FIG. 6, FIG. 7). Seven targets showed highly efficient (61.7-95.1%) mutagenesis while five showed poor (1.4-9.2%) mutagenesis. The most effective mutational positions were at 17-20 bases upstream of PAM, which was consistent with the previous study in higher organisms. The mutation frequency also varied depending on the length of the target sequence, as appreciated from the higher efficiency at galK_8 and galk_13, and lower at galK_9 and galK_11, of sgRNA with longer target sequences (FIG. 6, left bars).

To improve mutational efficiency, uracil DNA glycosylase inhibitor (UGI) from bacteriophage PBS2 (Zhigang, W. et al., Gene 99, 31-37 (1991).) and protein degradation tag (LVA-tag) (Andersen, J. B. et al., Appl. Environ. Microbiol. 64, 2240-2246 (1998).) were introduced by fusing at the C-terminus of dCas-CDA. UGI boosts the mutagenesis by cytidine deamination because it inhibits removal of uracil (immediate product of cytosine deamination) from DNA (non-patent literatures 10, 11). Use of the LVA-tag is expected to protect cells from injury and suppress the occurrence of escaper cells by decreasing the half-life of the dCas-CDA-UGI protein, which could be potentially harmful when expressed in excess. To assess the non-specific mutagenesis effect, whole genome sequencing analysis was performed on the cells expressing dCas, dCas-CDA and dCas-CDA-UGI-LVA. While dCas-CDA induced 0 to 2 SNV mutations, dCas-CDA-UGI-LVA induced 21-30 mutations with no positional bias in the entire genome (Table 3 and Table 4).

TABLE 3
Whole genome sequencing analysis of rifampicin resistance variant

| | | | | | Number | C:G |
|---|---|---|---|---|---|---|
| | | | rpoB mutation | | of | to |
| | | Sequence | | Amino | other | T:A |
| Strain | Clone | coverage | Position | acid | variants | SNV |
| dCas | 1 | 22.8 | 4172707 | S512F | 0 | 0 |
| dCas-CDA | 1 | 34.7 | 4172764 | S531F | 2 | 1 |
| | 2 | 28.1 | 4172718 | D516N | 0 | 0 |
| | 3 | 20.5 | 4172718 | D516N | 0 | 0 |
| dCas-CDA- | 1 | 17.3 | 4172737 | S522F | 21 | 21 |
| UGI-LVA | 2 | 23.3 | 4172737 | S522F | 30 | 29 |
| | 3 | 24.0 | 4172863 | P564L | 29 | 29 |

Rifampicin selected clones expressing each constructs (dCas, dCas-CDA or dCas-CDA-LVA-UGI) without sgRNA were subjected to whole genome sequencing. Biological triplicate was shown for dCas-CDA and dCas-CDA-LVA-UGI. Sequence coverage was calculated as sum base pair of mapped sequence over 4,631 Mopof Escherichia coli BW25113 genome sequence. List of unique mutation is shown in Table 4.

TABLE 4
List of unique SNV mutations
The detail of SNV detected by whole
genome sequencing is shown in Table 3.

| Strain_Clone | Region | Gene | Reference | Allele |
|---|---|---|---|---|
| dCas-CDA_1 | 883372 | rcdA | C | T |
| | 1371764 | ycjS | T | A |
| dCas-CDA-- | 31518 | carB | C | T |
| UGI-LVA_1 | 97137 | marG | C | T |
| | 290595 | intergenic | C | T |
| | 652855 | espE | C | T |
| | 689724 | miaB | C | T |
| | 896435 | artM | C | T |
| | 1267335 | intergenic | C | T |
| | 2054913 | nac | G | A |
| | 2062420 | yoaA | G | A |
| | 2072468 | intergenic | G | A |
| | 2110764 | wcaK | G | A |
| | 2550667 | intergenic | C | T |
| | 3322221 | intergenic | G | A |
| | 3681689 | yhjK | G | C |
| | 3692110 | bcsG | G | A |
| | 3983269 | hemC | C | T |
| | 4110323 | intergenic | G | A |
| | 4335067 | yjdF | C | T |
| | 4480068 | idnR | C | T |
| | 4596191 | tRNA-Leu | G | A |
| | 4596193 | tRNA-Leu | G | A |
| dCas-CDA-- | 800029 | ybhJ | C | T |
| UGI-LVA_2 | 1024591 | 23S rRNA | C | T |
| | 1050689 | torS | G | A |
| | 1199523 | ymfM | C | T |
| | 1644392 | ydfE | G | A |
| | 1723279 | intergenic | G | A |
| | 1728672 | intergenic | C | T |
| | 1761805 | ydiJ | G | A |
| | 2063321 | IS2 transposase | G | A |
| | 2072468 | intergenic | G | A |
| | 2208318 | mlrA | C | T |
| | 2245059 | nfo | C | T |
| | 2249454 | yeiL | G | A |
| | 2261905 | yeiR | G | A |
| | 2416291 | yfcI | A | C |
| | 2438584 | yfcA | G | A |
| | 2563453 | eutJ | G | A |
| | 2668799 | yphC | G | A |
| | 2957527 | ppdA | G | A |
| | 3168170 | parE | G | A |
| | 3365636 | nanT | G | A |
| | 3374392 | degQ | C | T |
| | 3441395 | 50S rRNA | G | A |
| | 3521736 | yrfF | G | A |
| | 3787020 | yibB | G | A |
| | 3921738 | viaA | C | T |
| | 4233085 | malG | C | T |
| | 4291989 | mdrO | C | T |
| | 4357531 | aspA | C | T |
| | 4486193 | intergenic | C | T |
| dCas-CDA-- | 124371 | intergenic | C | T |
| UGI-LVA_3 | 268728 | ykfC | C | T |
| | 701013 | nagE | C | T |
| | 1094697 | ycdX | C | T |
| | 1253681 | pth | C | T |
| | 1356138 | puuD | C | T |
| | 1513742 | curA | G | A |
| | 1552442 | ddpD | G | A |
| | 1771594 | ydtO | G | A |
| | 1950273 | torZ | G | A |
| | 2025444 | dcm | G | A |
| | 2221782 | mdtQ | G | A |
| | 2298519 | intergenic | G | A |
| | 2312704 | resC | G | A |
| | 2730338 | intergenic | C | T |
| | 2769113 | ypjI | G | A |
| | 2811281 | intergenic | G | A |
| | 3009344 | ygfK | G | A |
| | 3055007 | scpA | G | A |
| | 3542313 | malQ | G | A |
| | 3572324 | yhhW | G | A |
| | 3574775 | yhhY | G | A |
| | 3601990 | zniA | G | A |
| | 4111319 | hslU | G | A |
| | 4133803 | frwB | C | T |
| | 4347276 | codA | C | T |
| | 4384586 | unr | C | T |
| | 4386604 | omiB | C | T |
| | 4492813 | IS4 transposase | C | T |

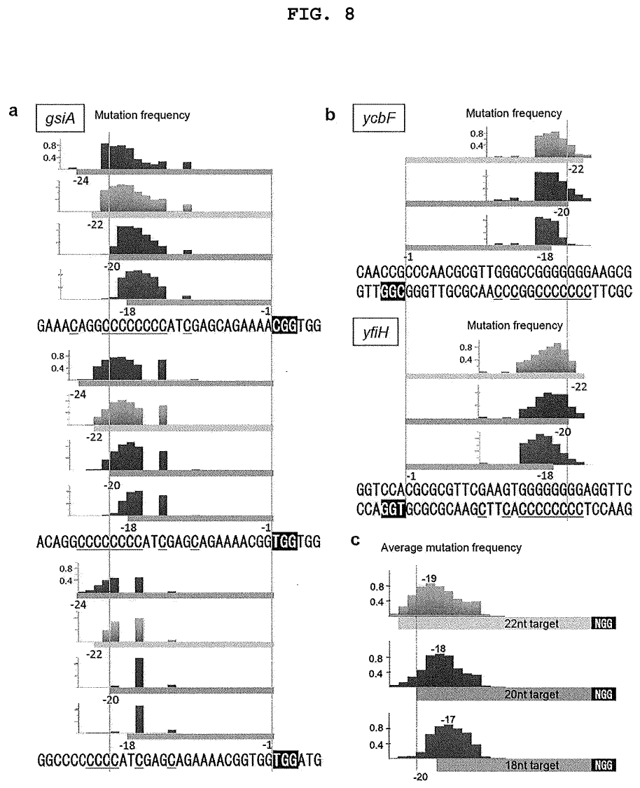

dCas-CDA-UGI-LVA showed robust mutagenesis at the all targeted sites regardless of the length and position of the target sequence (FIG. 5, right bars) and allowed for comparison of mutational spectra using different length of sgRNA. As a result, galK_9 and galk_11 showed extended mutational spectra towards 5′ ends (FIG. 6). To further characterize the effect of length of sgRNA target sequence, C-rich target sequences with 18nt, 20nt, 22nt and 24nt length were tested (FIGS. 8(a) and (b)). Mutational spectra for each of 5 target sites consistently showed the peak shift and expansion of window towards 5′ end as target sequence lengthened (FIG. 8(c)).

### Example 3 Multiplex Mutagenesis

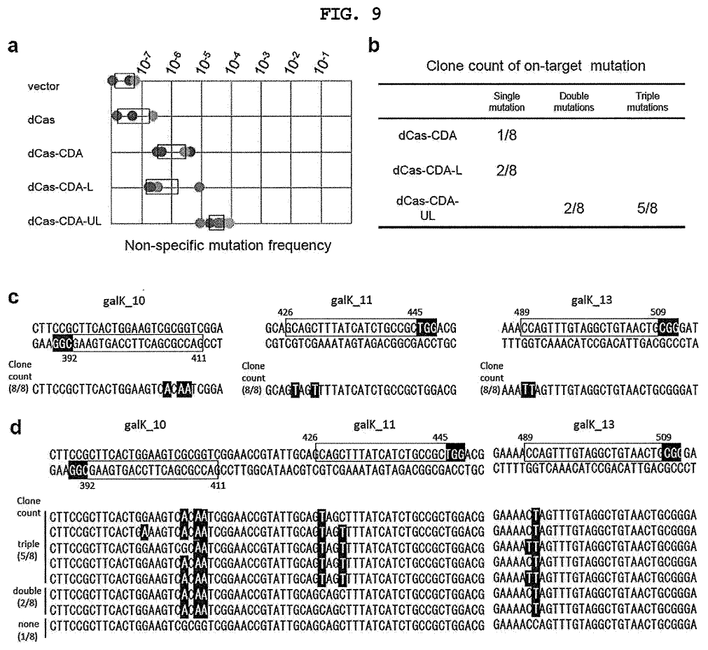

For multiplex editing, tandem repeat of sgRNA expression units was assembled onto a separate plasmid from the plasmid for alteration. A plasmid targeting three sites in the galK gene (galK_10, galK_11 and galK_13) was constructed and co-introduced into cells with the vector for alteration expressing dCas, dCas-CDA, dCas-CDA-LVA or dCas-CDA1-UGI-LVA. First, non-specific mutagenesis effect was assessed by analyzing occurrence of rifampicin resistance mutation (FIG. 9). While dCas-CDA showed approximately 10-fold increase over background mutational frequency, dCas-CDA-UGI-LVA showed another 10-fold increase over the mutational frequency of dCas-CDA. However, mutation at one site occurred in both and the mutation rate of at least the target did not show a significant difference between the presence or absence of LVA. Therefore, it was shown that non-specific mutagenesis can be suppressed by adding LVA, while maintaining mutation efficiency, and dCas-CDA and dCas-CDA-LVA were not efficient enough to obtain triple mutant at once, dCas-CDA-UGI-LVA succeeded in triple mutagenesis in five out of eight clones analyzed (FIGS. 9(b) and (d)), although mutational frequency seemed lower when compared with the result of each single targeting that yielded 100% (8/8) for each targets (FIGS. 9(c) and (d)). Therefore, it was shown that non-specific mutagenesis can be suppressed by a combination of UGI and LVA, while achieving high mutation efficiency.

Six different genes; galK, xylB (xylulokinase), manA (mannose-6-phosphate isomerase), pta (Phosphate acetyltransferase), adhE (Aldehyde-alcohol dehydrogenase), and tpiA (Triosephosphate isomerase) were then targeted to introduce stop codons (FIG. 10). Cells expressing dCas-CDA-UGI-LVA with six different genes targeting sgRNAs found seven out of eight clones were successfully mutated at all targeted loci (FIG. 10).

### Example 4 Multiple-Copy Gene Editing by Target-AID

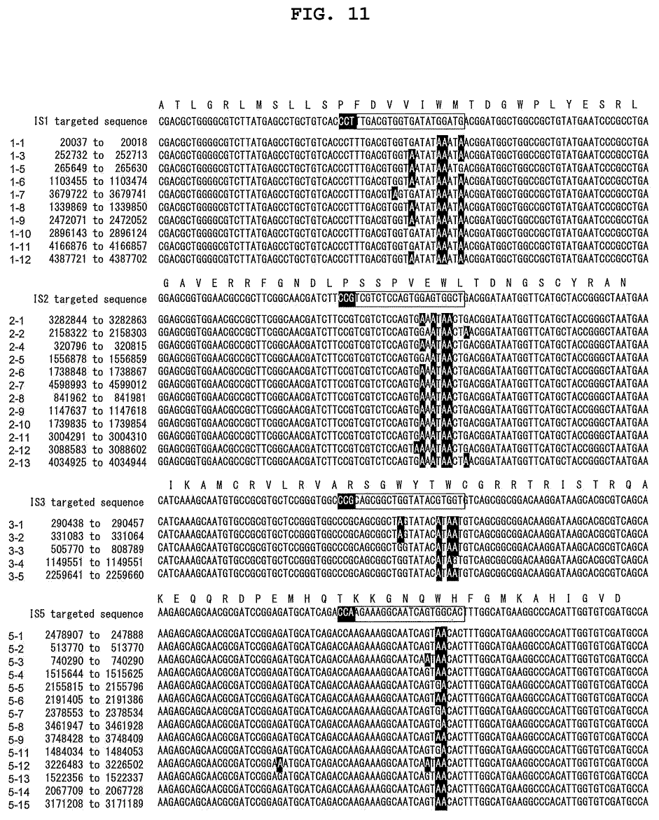

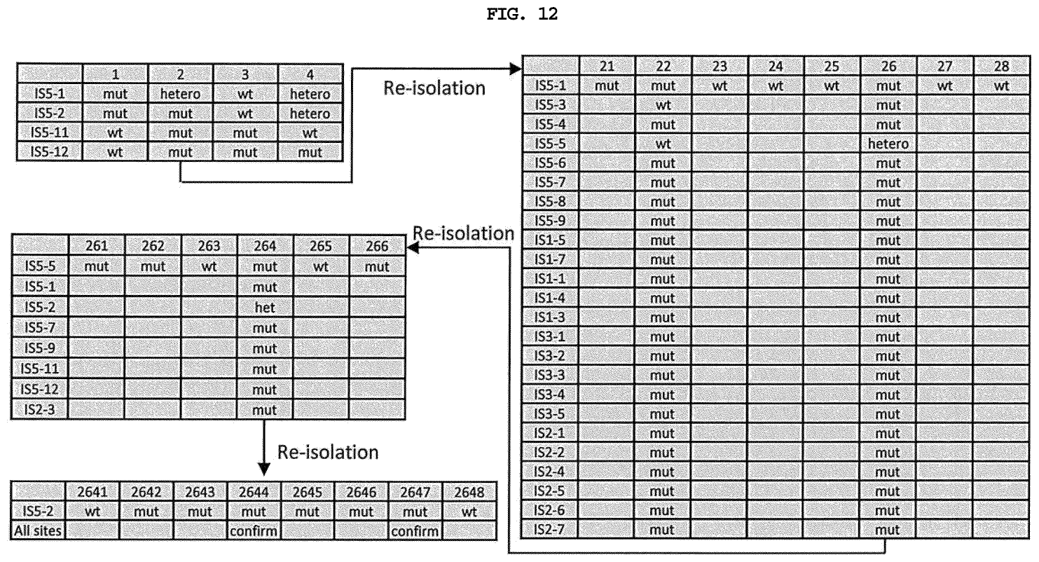

Multi-copy elements occupy a substantial amount of genome sequence. Unlike other methods that involves recombination or genomic cleavages, Target-AID may edit multiple loci at once using the same sgRNA sequence without inducing genomic instability. For a proof of concept, the four major transposable elements (TEs: IS1, 2, 3, and 5) in Escherichia coli genome were targeted simultaneously by using four sgRNAs. Ten, twelve, five and fourteen loci for IS1, 2, 3, and 5, respectively, were able to be amplified specifically by unique PCR primers for each locus. The sgRNAs were designed to contain the common sequences of the transposase genes of each TE to introduce stop codons (FIG. 11). Escherichia coli Top10 cells were transformed with two plasmids respectively expressing dCas-CDA-UGI-LVA and the four target sgRNAs. Isolation and verification procedure for IS-edited cell is depicted in FIG. 12 and described as follows. After double transformation and selection, colonies were PCR-amplified and sequenced at IS5-1, IS5-2, IS5-11, IS5-12 sites first. IS5 targets turned out to be less efficient. Out of four colonies analyzed, one contained three mutated sites and one heterogenic site (IS5-1). The cells were then suspended in liquid medium and spread onto plates to re-isolate colonies. Three out of eight colonies contained mutation at IS5-1, two of which were then further sequenced for the rest of 24 IS loci, showing that one contained all mutated sites but one incomplete, heterogenic site (IS5-5). The cells were then suspended and spread to obtain four out of six re-isolated clones that contain mutation at IS5-5. One of the clones was sequenced at IS5 sites and found to contain one heterogenic site (IS5-2). Eight clones were re-isolated and 6 contained mutation at IS5-2. Two of the clones were spread onto non-selection medium to obtain cells that lost plasmids. The cells were then genome-extracted and sequenced to confirm mutations at all the IS sites (FIG. 11) and further subjected to whole genome sequencing to assess genome-wide off-target effect. Among 34 potential off target sites from the reference genome that contain matched sequences at 1-8 bases proximal to the PAM, two sites were found to be mutated (Table 5).

TABLE 5
List of IS off-target candidate sites assessed by whole genome
sequencing.

| | | | Mis- | | SEQ | | | | |
|---|---|---|---|---|---|---|---|---|---|
| | | | | | ID | | | Frequency (%) | |
| Target | Region | Strand | match | Sequence | NO | PAM | gene | Clone_1 | Clone_2 |
| IS1 | | | 0 | CATCCATATCACCACGTCAA | 143 | AGG | insA | | |
| IS1 off-1 | 459398-459420 | − | 10 | GGTGGCATCGCCCACGTCAA | 144 | TGG | ybbA | <0.1 | <0.1 |
| IS1 off-2 | 2084230-2084252 | + | 6 | GTCCATTAGCACCACGTCAA | 145 | CGG | uvrY | 100 | 100 |
| IS1 off-3 | 2617074-2617096 | + | 11 | TGCAATCGCCTTCACGTCAA | 146 | TGG | yfeH | <0.1 | <0.1 |
| IS1 off-4 | 2965681-2965703 | + | 10 | ATAGCGGTAGGCCACGTCAA | 147 | TGG | cysN | <0.1 | <0.1 |
| IS1 off-5 | 3677105-3677127 | − | 11 | TGGGCGCTGTTGCACGTCAA | 148 | TGG | rimL | <0.1 | <0.1 |
| IS1 off-6 | 4615816-4615838 | + | 9 | ACCACCGCGTACCACGTCAA | 149 | TGG | fecA | <0.1 | <0.1 |
| IS2 | | | 0 | AGCCACTCCACTGGAGACGA | 150 | CGG | insD1 | | |
| IS2 off-1 | 2664330-2664352 | − | 9 | GATCTTGTGACCGGAGACGA | 151 | CGG | eutQ | <0.1 | <0.1 |
| IS2 off-2 | 2797547-2797569 | + | 9 | CCCGTTTTGCCAGGAGACGA | 152 | CGG | rseC | <0.1 | <0.1 |
| IS2 off-3 | 3478902-3478924 | | 6 | CATCTCTGCTCTGGAGACGA | 153 | TGG | degs | <0.1 | <0.1 |
| IS3 | | | 0 | ACCACGTATACCAGCCGCTG | 154 | CGG | insE1 | | |
| IS3 off-1 | 229782-229804 | − | 10 | AATTATCTCCACAGCCGCTG | 155 | TGG | pepD | <0.1 | <0.1 |
| IS3 off-2 | 297081-297103 | + | 9 | GAAGTGTTGATGAGCCGCTG | 156 | AGG | ykgE | <0.1 | <0.1 |
| IS3 off-3 | 828708-828730 | + | 9 | AAAAGCCGAGGCAGCCGCTG | 157 | CGG | tolA | <0.1 | <0.1 |
| IS3 off-4 | 992936-992958 | + | 8 | AACGTTGAAGTCAGCCGCTG | 158 | GGG | sers | <0.1 | <0.1 |
| IS3 off-5 | 1031011-1031033 | + | 10 | CAGAGCAGGTTCAGCCGCTG | 159 | CGG | mukB | <0.1 | <0.1 |
| IS3 off-6 | 2009292-2009314 | + | 7 | CATGCCAATATCAGCCGCTG | 160 | TGG | rsmL | 100 | 100 |
| IS3 off-7 | 2236079-2236101 | + | 7 | TCTGCTGCTGCCAGCCGCTG | 161 | AGG | alkA | <0.1 | <0.1 |
| IS3 off-8 | 2381447-2381469 | − | 8 | AAGAAGAGATCAAGCCGCTG | 162 | TGG | ccmH | <0.1 | <0.1 |
| IS3 off-9 | 2417471-2417493 | − | 9 | GACAACAGTCTTAGCCGCTG | 163 | GGG | yfaQ | <0.1 | <0.1 |
| IS3 off-10 | 2484639-2484661 | − | 9 | AGACATACAGCCAGCCGCTG | 164 | TGG | nuoL | <0.1 | <0.1 |
| IS3 off-11 | 2495424-2495446 | + | 10 | TTACTAAAAAAAAGCCGCTG | 165 | GGG | inter- | <0.1 | <0.1 |
| | | | | | | | genic | | |
| IS3 off-12 | 2495435-2495457 | − | 9 | GTGTAAAAAACGAGCCGCTG | 166 | GGG | inter- | <0.1 | <0.1 |
| | | | | | | | genic | | |
| IS3 off-13 | 2517513-2517535 | + | 9 | CGCATAGCTGGAAGCCGCTG | 167 | TGG | argT | <0.1 | <0.1 |
| IS3 off-14 | 2629660-2629682 | − | 8 | AATTAGTGGTGCAGCCGCTG | 168 | GGG | cysA | <0.1 | <0.1 |
| IS3 off-15 | 3232817-3232839 | − | 8 | TGCTGGTCTTTGAGCCGCTG | 169 | TGG | gss | <0.1 | <0.1 |
| IS3 off-16 | 3251435-3251457 | − | 10 | TTCCGGATAGTTAGCCGCTG | 170 | CGG | yqhD | <0.1 | <0.1 |
| IS3 off-17 | 3382350-3382372 | + | 7 | AGCATATCGTGCAGCCGCTG | 171 | GGG | agaI | <0.1 | <0.1 |
| IS3 off-18 | 3393244-3393266 | I | 8 | GGCGGGTTTTTTAGCCGCTG | 172 | GGG | yraQ | <0.1 | <0.1 |
| IS3 off-19 | 4109690-4109712 | I | 10 | CGGCTCGCTCGCAGCCGCTG | 173 | CGG | yigM | <0.1 | <0.1 |
| IS3 off-20 | 4378317-4378339 | + | 10 | CTGCGATTCCTCAGCCGCTG | 174 | GGG | yjcE | <0.1 | <0.1 |
| IS3 off-21 | 4418352-4418374 | + | 10 | ACACGTCGATATAGCCGCTG | 175 | CGG | phnI | <0.1 | <0.1 |
| IS5 | | | 0 | GTGCCACTGATTGCCTTTCT | 176 | TGG | insH1 | | |
| IS5 off-1 | 1632093-1632115 | − | 5 | GTACCTGTATTTGCCTTTCT | 177 | CGG | narU | <0.1 | <0.1 |
| IS5 off-2 | 3273904-3273926 | + | 10 | TTATCGGCCTGAGCCTTTCT | 178 | GGG | tolC | <0.1 | <0.1 |
| IS5 off-3 | 3775233-3775255 | − | 10 | CTTAACGTCCGCGCCTTTCT | 179 | CGG | kdgK | <0.1 | <0.1 |
| IS5 off-4 | 4553528-4553550 | + | 7 | CTGACCGATAATGCCTTTCT | 180 | TGG | ytfT | 1.2 | <0.1 |

Region indicates the target sites in DH10B database. Strand indicates orientation of the target sites. Probable off-target sequences were determined as described in the present specification. Mismatch indicates number of mismatches between on-target and off-target sequences. Mismatch nucleotides are highlighted in bold. Frequencies of C-to-T mutations at each sequence highlighted in italics are shown.

### Example 5 Comparison of Transformation Efficiency of Escherichia Coli by Vector for Yeast Expression

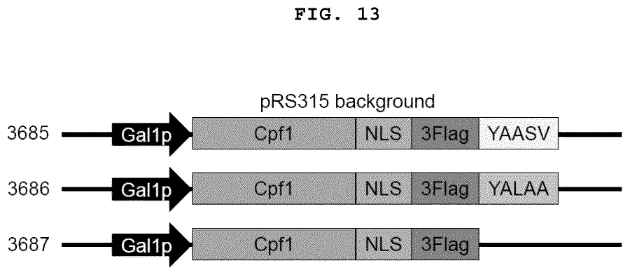

Vectors for yeast expression encoding LbCpf1 (SEQ ID NO: 326 and 327) as Cas effector protein, YAASV or YALAA as proteolysis tag (vector 3685: Cpf1-NLS-3xFlag-YAASV (SEQ ID NO: 328), vector 3687: Cpf1-NLS-3xFlag-YALAA (SEQ ID NO: 329)), and a control vector free of nucleic acid encoding proteolysis tag (vector 3687: Cpf1-NLS-3xFlag (SEQ ID NO: 330)) were generated using pRS315 vector as the base. Using these vectors, the transformation efficiency of Escherichia coli was verified. Schematic drawing of each vector is shown in FIG. 13. As shown in the following Table 6, a DNA solution containing each vector was adjusted to 2 ng/μl, 20 μl of Escherichia coli Top10 competent cells were transform by adding 1 μl (2 ng) of the DNA solution. Thereafter, 200 μl of SOC was added, the mixture was recovery cultured at 37° C. for 1 hr and stood on ice for 5 min to discontinue proliferation, and 1 μl of 50 mg/ml Amp was added. A part of the culture medium (1 μl or 10 μl) was diluted with TE, applied onto LB+Amp plate, cultured overnight at 37° C. and the number of colonies was counted. The results are shown in Table 6.

TABLE 6

| | 3685 | 3686 | 3687 |
|---|---|---|---|
| Plasmid conc (ng/ul) | 13 | 13.5 | 17 |
| Dilution(to 2 ng/ul) | 3.1 + 16.9 | 3.0 + 17 | 2.4 + 17.6 |
| Colony in 10 ul | 39 | 96 | 28 |
| Colony in 1 ul | 3 | 4 | 1 |

It was shown that the transformation efficiency of Escherichia coli was high when vector 3685 and vector 3686 having a nucleic acid encoding a proteolysis tag were used, as compared to the use of the control vector 3687. Therefore, use of a proteolysis tag is expected to improve replication efficiency when replicating vectors in bacteria such as Escherichia coli and the like even if the vector is for expressing a heterologous organism.

This application is based on Japanese patent application No. 2017-225221 filed in Japan (filing date: Nov. 22, 2017), the contents of which are encompassed in full herein.

### INDUSTRIAL APPLICABILITY

According to the present invention, a vector which is stable and amplifiable even in a host bacterium and has low toxicity, and a complex for genome editing which is encoded by the vector are provided. According to the method for genome editing using the nucleic acid and nucleic acid altering enzyme of the present invention, it is possible to alter the gene of a host bacterium while suppressing non-specific mutation and the like. Since this method does not rely on host dependent factors such as RecA, it can be applied to a wide range of bacteria, and is extremely useful.

1. A complex comprising a nucleic acid sequence-recognizing module and a proteolysis tag,
wherein the module is linked to the proteolysis tag, the module specifically binds to a target nucleotide sequence in a double stranded DNA,
wherein the complex is further bound with a nucleic acid altering enzyme that converts one or more nucleotides in the target nucleotide sequence to a different nucleotide, deletes one or more nucleotides in the target nucleotide sequence, or inserts one or more nucleotides in the target nucleotide sequence,
wherein the nucleic acid altering enzyme is a deaminase or a DNA glycosylase, and
wherein the nucleic acid sequence-recognizing module is selected from the group consisting of CRISPR-mutant Cas, zinc finger motif, TAL effector and PPR motif.

2. A method for altering a targeted site of a double stranded DNA of a bacterium, or regulating an expression of a gene encoded by a double-stranded DNA near the site, comprising a step of bringing a complex comprising a nucleic acid sequence-recognizing module specifically binding to a target nucleotide sequence in a selected double stranded DNA and a proteolysis tag at the C-terminal of the module.

3. A method for efficient production of transformant with the replicating vector for expressing in a heterologous organism, wherein the method comprises the steps of:
providing a vector which encodes the complex comprising a nucleic acid sequence-recognizing module specifically binding to a target nucleotide sequence in a selected double stranded DNA of the heterologous organism and a proteolysis tag at the C terminal of the module;
introducing the vector into a host organism; and
subjecting the host organism to a condition for proliferation.

4. The method according to claim 3, wherein the method further comprises selecting a transformant from the host organisms, which shows efficient proliferation.

5. The method according to claim 3, wherein the transformant is a bacterium.

6. The method according to claim 3, wherein the heterologous organism is eukaryotic.

7. A transformant with the replicating vector for expressing in a heterologous organism wherein the vector encodes the complex comprising a nucleic acid sequence-recognizing module specifically binding to a target nucleotide sequence in a selected double stranded DNA of the heterologous organism and a proteolysis tag at the C-terminal of the module.


SEQUENCE LISTING (340 sequences)

SEQ ID NO: 1 - DNA, 4104 bp, Streptococcus pyogenes
  variation 28..30: note = mutation -
  variation 2518..2520: note = mutation -
  source 1..4104: mol_type = other DNA
  CDS 1..4104: note = dCas9 (D10A and H840A)
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 2 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - DNA, 624 bp, Dermocystidium marinum
  source 1..624: mol_type = other DNA; note = Perkinsus marinus
  CDS 1..624: note = PmCDA1
atgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaatctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtt

SEQ ID NO: 4 - protein, 208 aa, Dermocystidium marinum
  source 1..208: note = Perkinsus marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 5 - DNA, 363 bp, synthetic construct
  misc_feature 1..363: note = Synthetic Sequence - linker region
  misc_feature 31..201: note = SH3 domain
  misc_feature 208..273: note = 3xFLAG tag
  misc_feature 292..357: note = 3xFLAG tag
  source 1..363: mol_type = other DNA
  CDS 1..363
ggtggaggaggttctggaggtggaggttctgctgagtatgtgcgagccctctttgactttaatgggaatgatgaagaggatcttccctttaagaaaggagacatcctgagaatccgggataagcctgaggagcagtggtggaatgcagaggacagcgaaggaaagagggggatgattcctgtcccttacgtggagaagtattccggagactataaggaccacgacggagactacaaggatcatgatattgattacaaagacgatgacgataagtctaggctcgagtccggagactataaggaccacgacggagactacaaggatcatgatattgattacaaagacgatgacgataagtctagg

SEQ ID NO: 6 - protein, 121 aa, synthetic construct
  REGION 1..121: note = Synthetic Construct
GGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRLESGDYKDHDGDYKDHDIDYKDDDDKSR

SEQ ID NO: 7 - DNA, 171 bp, synthetic construct
  misc_feature 1..171: note = Synthetic Sequence - SH3 domain
  source 1..171: mol_type = other DNA
  CDS 1..171
gctgagtatgtgcgagccctctttgactttaatgggaatgatgaagaggatcttccctttaagaaaggagacatcctgagaatccgggataagcctgaggagcagtggtggaatgcagaggacagcgaaggaaagagggggatgattcctgtcccttacgtggagaagtat

SEQ ID NO: 8 - protein, 57 aa, synthetic construct
  REGION 1..57: note = Synthetic Construct
AEYVRALFDFNGNDEEDLPFKKGDILRIRDKPEEQWWNAEDSEGKRGMIPVPYVEKY

SEQ ID NO: 9 - DNA, 66 bp, synthetic construct
  misc_feature 1..66: note = Synthetic Sequence - 3xFLAG tag
  source 1..66: mol_type = other DNA
  CDS 1..66
gactataaggaccacgacggagactacaaggatcatgatattgattacaaagacgatgacgataag

SEQ ID NO: 10 - protein, 22 aa, synthetic construct
  REGION 1..22: note = Synthetic Construct
DYKDHDGDYKDHDIDYKDDDDK

SEQ ID NO: 11 - DNA, 24 bp, synthetic construct
  misc_feature 1..24: note = Synthetic Sequence - FLAG tag
  source 1..24: mol_type = other DNA
  CDS 1..24
gattacaaagacgatgacgataag

SEQ ID NO: 12 - protein, 8 aa, synthetic construct
  REGION 1..8: note = Synthetic Construct
DYKDDDDK

SEQ ID NO: 13 - DNA, 252 bp, Myocastor coypus
  source 1..252: mol_type = other DNA
  CDS 1..252: note = PBS2-derived UGI
atgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgcta

SEQ ID NO: 14 - protein, 84 aa, Myocastor coypus
MTNLSDIIEKETGKQLVIQESILMLPEEVEEVIGNKPESDILVHTAYDESTDENVMLLTSDAPEYKPWALVIQDSNGENKIKML

SEQ ID NO: 15 - DNA, 484 bp, synthetic construct
  misc_feature 1..484: note = synthetic oligonucleotide: J23119-sgRNA-units
  regulatory 59..93: note = promoter - J23119 promoter; regulatory_class = promoter
  misc_structure 94..113: note = minimal CRISPR array
  misc_structure 114..196: note = gRNA scaffold
  regulatory 269..303: note = promoter - J23119 promoter; regulatory_class = promoter
  misc_structure 304..323: note = minimal CRISPR array
  misc_structure 324..406: note = gRNA scaffold
  source 1..484: mol_type = other DNA
gataacaattgagcaagaatcttcatcgatgcatcagaaaattattttaaatttcctcttgacagctagctcagtcctaggtataatgctagcagagacccgggatggtctcagttttagagctagaaatagcaagttaaaataaggctagtccgttatcaacttgaaaaagtggcaccgagtcggtgctttttttccgaaaaaaaaaccccgcccctgacagggcggggttttttttaattaacagaaaattattttaaatttcctcttgacagctagctcagtcctaggtataatgctagcagaagagccggcgctcttcagttttagagctagaaatagcaagttaaaataaggctagtccgttatcaacttgaaaaagtggcaccgagtcggtgctttttttccgaaaaaaaaaccccgcccctgacagggcggggtttttttttctagaccgcaaaaatcagcgcgcaaatacgcatac

SEQ ID NO: 16 - DNA, 35 bp, synthetic construct
  misc_feature 1..35: note = Synthetic Sequence - J23119 promoter
  source 1..35: mol_type = other DNA
ttgacagctagctcagtcctaggtataatgctagc

SEQ ID NO: 17 - DNA, 20 bp, Streptococcus pyogenes
  misc_feature 1..20: note = minimal CRISPR array
  source 1..20: mol_type = other DNA
agagacccgggatggtctca

SEQ ID NO: 18 - DNA, 83 bp, Streptococcus pyogenes
  misc_structure 1..83: note = gRNA scaffold
  source 1..83: mol_type = other DNA
gttttagagctagaaatagcaagttaaaataaggctagtccgttatcaacttgaaaaagtggcaccgagtcggtgcttttttt

SEQ ID NO: 19 - RNA, 19 nt, Streptococcus pyogenes
  misc_structure 1..19: note = crRNA direct repeat sequence.
  source 1..19: mol_type = other RNA
aatttctactgttgtagat

SEQ ID NO: 20 - DNA, 74 bp, synthetic construct
  misc_feature 1..74: note = PCR Primer
  source 1..74: mol_type = other DNA
gctactctagaaaaaaaaaccccgccctgtcaggggcggggtttttttttcggggtttagcaagatggcagcgc

SEQ ID NO: 21 - DNA, 91 bp, synthetic construct
  misc_feature 1..91: note = PCR Primer
  source 1..91: mol_type = other DNA
gcctgatcgatgcatcagaaaattattttaaatttcctcttgacagctagctcagtcctaggtataatgctagcagagacccgggatggtc

SEQ ID NO: 22 - DNA, 52 bp, synthetic construct
  misc_feature 1..52: note = PCR Primer
  source 1..52: mol_type = other DNA
gatcctttttgataatctcgtcgacataacaattgagcaagaatcttcatcg

SEQ ID NO: 23 - DNA, 51 bp, synthetic construct
  misc_feature 1..51: note = PCR Primer
  source 1..51: mol_type = other DNA
ctcacgttaagggattttggtcatctgcagtgtatgcgtatttgcgcgctg

SEQ ID NO: 24 - DNA, 57 bp, synthetic construct
  misc_feature 1..57: note = PCR Primer
  source 1..57: mol_type = other DNA
gaagattcttgctcaattgttatgtcgacgagattatcaaaaaggatcttcacctag

SEQ ID NO: 25 - DNA, 51 bp, synthetic construct
  misc_feature 1..51: note = PCR Primer
  source 1..51: mol_type = other DNA
cagcgcgcaaatacgcatacactgcagatgaccaaaatcccttaacgtgag

SEQ ID NO: 26 - DNA, 20 bp, synthetic construct
  misc_feature 1..20: note = PCR Primer
  source 1..20: mol_type = other DNA
ctgcacgcgcacttttatcc

SEQ ID NO: 27 - DNA, 21 bp, synthetic construct
  misc_feature 1..21: note = PCR Primer
  source 1..21: mol_type = other DNA
ctctgtttgccaacgcatttg

SEQ ID NO: 28 - DNA, 20 bp, synthetic construct
  misc_feature 1..20: note = PCR Primer
  source 1..20: mol_type = other DNA
caatggtgacatcacgcagg

SEQ ID NO: 29 - DNA, 21 bp, synthetic construct
  misc_feature 1..21: note = PCR Primer
  source 1..21: mol_type = other DNA
cctgggcgataacgtagttgc

SEQ ID NO: 30 - DNA, 20 bp, synthetic construct
  misc_feature 1..20: note = PCR Primer
  source 1..20: mol_type = other DNA
cctcggcaaccgtcgtatcc

SEQ ID NO: 31 - DNA, 20 bp, synthetic construct
  misc_feature 1..20: note = PCR Primer
  source 1..20: mol_type = other DNA
ccggttatcggtagcgatac

SEQ ID NO: 32 - DNA, 20 bp, synthetic construct
  misc_feature 1..20: note = PCR Primer
  source 1..20: mol_type = other DNA
gcctggggattattgtgtgg

SEQ ID NO: 33 - DNA, 19 bp, synthetic construct
  misc_feature 1..19: note = PCR Primer
  source 1..19: mol_type = other DNA
gcggctccaggttacttcc

SEQ ID NO: 34 - DNA, 17 bp, synthetic construct
  misc_feature 1..17: note = PCR Primer
  source 1..17: mol_type = other DNA
gccatcacttccagcgc

SEQ ID NO: 35 - DNA, 18 bp, synthetic construct
  misc_feature 1..18: note = PCR Primer
  source 1..18: mol_type = other DNA
gccaaatcggcggtaacg

SEQ ID NO: 36 - DNA, 20 bp, synthetic construct
  misc_feature 1..20: note = PCR Primer
  source 1..20: mol_type = other DNA
ccatttcgtaaccgccagtc

SEQ ID NO: 37 - DNA, 20 bp, synthetic construct
  misc_feature 1..20: note = PCR Primer
  source 1..20: mol_type = other DNA
ctctcgtattcgagcagatg

SEQ ID NO: 38 - DNA, 17 bp, synthetic construct
  misc_feature 1..17: note = PCR Primer
  source 1..17: mol_type = other DNA
ccgcccatagcaaccag

SEQ ID NO: 39 - DNA, 16 bp, synthetic construct
  misc_feature 1..16: note = PCR Primer
  source 1..16: mol_type = other DNA
ggggcggccatcttcc

SEQ ID NO: 40 - DNA, 18 bp, synthetic construct
  misc_feature 1..18: note = PCR Primer
  source 1..18: mol_type = other DNA
cagccgaggaaattcagg

SEQ ID NO: 41 - DNA, 49 bp, synthetic construct
  misc_feature 1..49: note = PCR Primer
  source 1..49: mol_type = other DNA
tctttccctacacgacgctcttccgatctccgcagaacaggcagcagag

SEQ ID NO: 42 - DNA, 54 bp, synthetic construct
  misc_feature 1..54: note = PCR Primer
  source 1..54: mol_type = other DNA
gtgactggagttcagacgtgtgctcttccgatctgacaatgggcgcatcgaggg

SEQ ID NO: 43 - DNA, 49 bp, synthetic construct
  misc_feature 1..49: note = PCR Primer
  source 1..49: mol_type = other DNA
tctttccctacacgacgctcttccgatctcacaccgactacaacgacgg

SEQ ID NO: 44 - DNA, 54 bp, synthetic construct
  misc_feature 1..54: note = PCR Primer
  source 1..54: mol_type = other DNA
gtgactggagttcagacgtgtgctcttccgatctgctgctgcaatacggttccg

SEQ ID NO: 45 - DNA, 49 bp, synthetic construct
  misc_feature 1..49: note = PCR Primer
  source 1..49: mol_type = other DNA
tctttccctacacgacgctcttccgatctcttcggcggcgtggacatgg

SEQ ID NO: 46 - DNA, 55 bp, synthetic construct
  misc_feature 1..55: note = PCR Primer
  source 1..55: mol_type = other DNA
gtgactggagttcagacgtgtgctcttccgatctacgacagccacacctttgggc

SEQ ID NO: 47 - DNA, 52 bp, synthetic construct
  misc_feature 1..52: note = PCR Primer
  source 1..52: mol_type = other DNA
tctttccctacacgacgctcttccgatctggcggtggaaacgggtaccgtcg

SEQ ID NO: 48 - DNA, 54 bp, synthetic construct
  misc_feature 1..54: note = PCR Primer
  source 1..54: mol_type = other DNA
gtgactggagttcagacgtgtgctcttccgatctcgcccaccagcgatagcgtt

SEQ ID NO: 49 - DNA, 49 bp, synthetic construct
  misc_feature 1..49: note = PCR Primer
  source 1..49: mol_type = other DNA
tctttccctacacgacgctcttccgatctggcgctcttgattttcgccg

SEQ ID NO: 50 - DNA, 54 bp, synthetic construct
  misc_feature 1..54: note = PCR Primer
  source 1..54: mol_type = other DNA
gtgactggagttcagacgtgtgctcttccgatctggatggcacgctggcaatgc

SEQ ID NO: 51 - DNA, 49 bp, synthetic construct
  misc_feature 1..49: note = PCR Primer
  source 1..49: mol_type = other DNA
tctttccctacacgacgctcttccgatctgcctccctgtgctgttttgc

SEQ ID NO: 52 - DNA, 54 bp, synthetic construct
  misc_feature 1..54: note = PCR Primer
  source 1..54: mol_type = other DNA
gtgactggagttcagacgtgtgctcttccgatctgctcaacacccacgttcgcc

SEQ ID NO: 53 - DNA, 20 bp, synthetic construct
  misc_feature 1..20: note = PCR Primer
  source 1..20: mol_type = other DNA
gctcattatttgcccgcttg

SEQ ID NO: 54 - DNA, 19 bp, synthetic construct
  misc_feature 1..19: note = PCR Primer
  source 1..19: mol_type = other DNA
tgccggttgccagatagtc

SEQ ID NO: 55 - DNA, 19 bp, synthetic construct
  misc_feature 1..19: note = PCR Primer
  source 1..19: mol_type = other DNA
ggtcttcaggaaatcaccg

SEQ ID NO: 56 - DNA, 20 bp, synthetic construct
  misc_feature 1..20: note = PCR Primer
  source 1..20: mol_type = other DNA
cgttcaaccacttcagtgtc

SEQ ID NO: 57 - DNA, 20 bp, synthetic construct
  misc_feature 1..20: note = PCR Primer
  source 1..20: mol_type = other DNA
cacaaagctgtaaatcagcg

SEQ ID NO: 58 - DNA, 20 bp, synthetic construct
  misc_feature 1..20: note = PCR Primer
  source 1..20: mol_type = other DNA
gtcaatgcaacacccctttc

SEQ ID NO: 59 - DNA, 20 bp, synthetic construct
  misc_feature 1..20: note = PCR Primer
  source 1..20: mol_type = other DNA
ctacaaccaggtcgagtcag

SEQ ID NO: 60 - DNA, 21 bp, synthetic construct
  misc_feature 1..21: note = PCR Primer
  source 1..21: mol_type = other DNA
gtaatcctgcacctccatcac

SEQ ID NO: 61 - DNA, 20 bp, synthetic construct
  misc_feature 1..20: note = PCR Primer
  source 1..20: mol_type = other DNA
gccagtagtacccgtcgttg

SEQ ID NO: 62 - DNA, 21 bp, synthetic construct
  misc_feature 1..21: note = PCR Primer
  source 1..21: mol_type = other DNA
cacaagtcgtatttccagagg

SEQ ID NO: 63 - DNA, 20 bp, synthetic construct
  misc_feature 1..20: note = PCR Primer
  source 1..20: mol_type = other DNA
ctgcaataagcagaaccacc

SEQ ID NO: 64 - DNA, 20 bp, synthetic construct
  misc_feature 1..20: note = PCR Primer
  source 1..20: mol_type = other DNA
tgttgtgcggtaagtgtctg

SEQ ID NO: 65 - DNA, 21 bp, synthetic construct
  misc_feature 1..21: note = PCR Primer
  source 1..21: mol_type = other DNA
gagcaatggatggattcgaag

SEQ ID NO: 66 - DNA, 22 bp, synthetic construct
  misc_feature 1..22: note = PCR Primer
  source 1..22: mol_type = other DNA
tgaacaactgtccatgatttcg

SEQ ID NO: 67 - DNA, 19 bp, synthetic construct
  misc_feature 1..19: note = PCR Primer
  source 1..19: mol_type = other DNA
ggtactttccgggcaaccg

SEQ ID NO: 68 - DNA, 18 bp, synthetic construct
  misc_feature 1..18: note = PCR Primer
  source 1..18: mol_type = other DNA
ctgccattagcgcagcca

SEQ ID NO: 69 - DNA, 19 bp, synthetic construct
  misc_feature 1..19: note = PCR Primer
  source 1..19: mol_type = other DNA
catagctctacacgccagg

SEQ ID NO: 70 - DNA, 22 bp, synthetic construct
  misc_feature 1..22: note = PCR Primer
  source 1..22: mol_type = other DNA
atcatgggctccttttagttgc

SEQ ID NO: 71 - DNA, 19 bp, synthetic construct
  misc_feature 1..19: note = PCR Primer
  source 1..19: mol_type = other DNA
gatattgcccgccggacac

SEQ ID NO: 72 - DNA, 18 bp, synthetic construct
  misc_feature 1..18: note = PCR Primer
  source 1..18: mol_type = other DNA
cgatctaaagcgcgcagc

SEQ ID NO: 73 - DNA, 22 bp, synthetic construct
  misc_feature 1..22: note = PCR Primer
  source 1..22: mol_type = other DNA
gacgttgttgaaaatgtagggt

SEQ ID NO: 74 - DNA, 21 bp, synthetic construct
  misc_feature 1..21: note = PCR Primer
  source 1..21: mol_type = other DNA
gcctaacgcctttaattcagg

SEQ ID NO: 75 - DNA, 19 bp, synthetic construct
  misc_feature 1..19: note = PCR Primer
  source 1..19: mol_type = other DNA
tgttgtggagcctgaacgg

SEQ ID NO: 76 - DNA, 19 bp, synthetic construct
  misc_feature 1..19: note = PCR Primer
  source 1..19: mol_type = other DNA
gcaactgttccggcagatg

SEQ ID NO: 77 - DNA, 23 bp, synthetic construct
  misc_feature 1..23: note = PCR Primer
  source 1..23: mol_type = other DNA
cggattaatgataagtggatcag

SEQ ID NO: 78 - DNA, 21 bp, synthetic construct
  misc_feature 1..21: note = PCR Primer
  source 1..21: mol_type = other DNA
cttagtgaatatttgccgacg

SEQ ID NO: 79 - DNA, 22 bp, synthetic construct
  misc_feature 1..22: note = PCR Primer
  source 1..22: mol_type = other DNA
gctgataagttacctcctgacc

SEQ ID NO: 80 - DNA, 22 bp, synthetic construct
  misc_feature 1..22: note = PCR Primer
  source 1..22: mol_type = other DNA
gcgactatacaggttattgacc

SEQ ID NO: 81 - DNA, 20 bp, synthetic construct
  misc_feature 1..20: note = PCR Primer
  source 1..20: mol_type = other DNA
acattacagagaagccgatg

SEQ ID NO: 82 - DNA, 19 bp, synthetic construct
  misc_feature 1..19: note = PCR Primer
  source 1..19: mol_type = other DNA
gtgatagttagcgatgccg

SEQ ID NO: 83 - DNA, 22 bp, synthetic construct
  misc_feature 1..22: note = PCR Primer
  source 1..22: mol_type = other DNA
ggacgaataaacgcataattac

SEQ ID NO: 84 - DNA, 19 bp, synthetic construct
  misc_feature 1..19: note = PCR Primer
  source 1..19: mol_type = other DNA
tcccaaccttctgtcacag

SEQ ID NO: 85 - DNA, 19 bp, synthetic construct
  misc_feature 1..19: note = PCR Primer
  source 1..19: mol_type = other DNA
caattttcgcaccggaatc

SEQ ID NO: 86 - DNA, 20 bp, synthetic construct
  misc_feature 1..20: note = PCR Primer
  source 1..20: mol_type = other DNA
atggagatacgacaatcagc

SEQ ID NO: 87 - DNA, 21 bp, synthetic construct
  misc_feature 1..21: note = PCR Primer
  source 1..21: mol_type = other DNA
caattcctggaacattatccg

SEQ ID NO: 88 - DNA, 19 bp, synthetic construct
  misc_feature 1..19: note = PCR Primer
  source 1..19: mol_type = other DNA
tgagtgatgttttggcgac

SEQ ID NO: 89 - DNA, 19 bp, synthetic construct
  misc_feature 1..19: note = PCR Primer
  source 1..19: mol_type = other DNA
ctgtactcacagggtgatg

SEQ ID NO: 90 - DNA, 18 bp, synthetic construct
  misc_feature 1..18: note = PCR Primer
  source 1..18: mol_type = other DNA
ggcagacagtttgaaacc

SEQ ID NO: 91 - DNA, 19 bp, synthetic construct
  misc_feature 1..19: note = PCR Primer
  source 1..19: mol_type = other DNA
cgccacgaacgtagttagc

SEQ ID NO: 92 - DNA, 22 bp, synthetic construct
  misc_feature 1..22: note = PCR Primer
  source 1..22: mol_type = other DNA
gattggtgaacacaccgactac

SEQ ID NO: 93 - DNA, 20 bp, synthetic construct
  misc_feature 1..20: note = PCR Primer
  source 1..20: mol_type = other DNA
ggtcaggtggtttggaaagc

SEQ ID NO: 94 - DNA, 21 bp, synthetic construct
  misc_feature 1..21: note = PCR Primer
  source 1..21: mol_type = other DNA
aagtggacacgctatacctgc

SEQ ID NO: 95 - DNA, 20 bp, synthetic construct
  misc_feature 1..20: note = PCR Primer
  source 1..20: mol_type = other DNA
cactcaacacataccgtgcc

SEQ ID NO: 96 - DNA, 21 bp, synthetic construct
  misc_feature 1..21: note = PCR Primer
  source 1..21: mol_type = other DNA
caacaccaaactggaacacgg

SEQ ID NO: 97 - DNA, 22 bp, synthetic construct
  misc_feature 1..22: note = PCR Primer
  source 1..22: mol_type = other DNA
cggaggaaacagaatcagtgtg

SEQ ID NO: 98 - DNA, 21 bp, synthetic construct
  misc_feature 1..21: note = PCR Primer
  source 1..21: mol_type = other DNA
gagatggtggagatcctctcg

SEQ ID NO: 99 - DNA, 19 bp, synthetic construct
  misc_feature 1..19: note = PCR Primer
  source 1..19: mol_type = other DNA
gatagttagcgatgccggg

SEQ ID NO: 100 - DNA, 22 bp, synthetic construct
  misc_feature 1..22: note = PCR Primer
  source 1..22: mol_type = other DNA
ggagaatccccaggttatctgg

SEQ ID NO: 101 - DNA, 20 bp, synthetic construct
  misc_feature 1..20: note = PCR Primer
  source 1..20: mol_type = other DNA
tgaaacgtgcgggtctcaac

SEQ ID NO: 102 - DNA, 23 bp, synthetic construct
  misc_feature 1..23: note = PCR Primer
  source 1..23: mol_type = other DNA
ggatagtggttaatggtggcgtc

SEQ ID NO: 103 - DNA, 20 bp, synthetic construct
  misc_feature 1..20: note = PCR Primer
  source 1..20: mol_type = other DNA
cgtgctgagggctatttacc

SEQ ID NO: 104 - DNA, 22 bp, synthetic construct
  misc_feature 1..22: note = PCR Primer
  source 1..22: mol_type = other DNA
gacgtcatcatttagccagatg

SEQ ID NO: 105 - DNA, 22 bp, synthetic construct
  misc_feature 1..22: note = PCR Primer
  source 1..22: mol_type = other DNA
ggttctcaggttaatgtttcgg

SEQ ID NO: 106 - DNA, 20 bp, synthetic construct
  misc_feature 1..20: note = PCR Primer
  source 1..20: mol_type = other DNA
caccagatactacgttaccg

SEQ ID NO: 107 - DNA, 20 bp, synthetic construct
  misc_feature 1..20: note = PCR Primer
  source 1..20: mol_type = other DNA
ttcggactgaaaggagcaag

SEQ ID NO: 108 - DNA, 20 bp, synthetic construct
  misc_feature 1..20: note = PCR Primer
  source 1..20: mol_type = other DNA
agattcgtgctcacctttcc

SEQ ID NO: 109 - DNA, 22 bp, synthetic construct
  misc_feature 1..22: note = PCR Primer
  source 1..22: mol_type = other DNA
gattagtattggcgctgttgtg

SEQ ID NO: 110 - DNA, 21 bp, synthetic construct
  misc_feature 1..21: note = PCR Primer
  source 1..21: mol_type = other DNA
cagtccatttcaccgtatgag

SEQ ID NO: 111 - DNA, 21 bp, synthetic construct
  misc_feature 1..21: note = PCR Primer
  source 1..21: mol_type = other DNA
acagacgaccagagtaatgtc

SEQ ID NO: 112 - DNA, 19 bp, synthetic construct
  misc_feature 1..19: note = PCR Primer
  source 1..19: mol_type = other DNA
tggttacgcgctttcatgg

SEQ ID NO: 113 - DNA, 21 bp, synthetic construct
  misc_feature 1..21: note = PCR Primer
  source 1..21: mol_type = other DNA
caggctgaacatggataagac

SEQ ID NO: 114 - DNA, 22 bp, synthetic construct
  misc_feature 1..22: note = PCR Primer
  source 1..22: mol_type = other DNA
acgtatggacatctaaacatcc

SEQ ID NO: 115 - DNA, 21 bp, synthetic construct
  misc_feature 1..21: note = PCR Primer
  source 1..21: mol_type = other DNA
gcaaggttgtgcttctaaagg

SEQ ID NO: 116 - DNA, 23 bp, synthetic construct
  misc_feature 1..23: note = PCR Primer
  source 1..23: mol_type = other DNA
cctgcaatctaaaggtaaggatc

SEQ ID NO: 117 - DNA, 20 bp, synthetic construct
  misc_feature 1..20: note = PCR Primer
  source 1..20: mol_type = other DNA
gattgctgtggcaggtttac

SEQ ID NO: 118 - DNA, 20 bp, synthetic construct
  misc_feature 1..20: note = PCR Primer
  source 1..20: mol_type = other DNA
cagtacaacctagttgcacc

SEQ ID NO: 119 - DNA, 20 bp, synthetic construct
  misc_feature 1..20: note = PCR Primer
  source 1..20: mol_type = other DNA
tgaagattccgtgcgtaacc

SEQ ID NO: 120 - DNA, 19 bp, synthetic construct
  misc_feature 1..19: note = PCR Primer
  source 1..19: mol_type = other DNA
cacgatgaaaccgtcagtg

SEQ ID NO: 121 - DNA, 19 bp, synthetic construct
  misc_feature 1..19: note = PCR Primer
  source 1..19: mol_type = other DNA
atgctactgccggaacaac

SEQ ID NO: 122 - DNA, 20 bp, synthetic construct
  misc_feature 1..20: note = PCR Primer
  source 1..20: mol_type = other DNA
tgatgtcagcgagaagatgg

SEQ ID NO: 123 - DNA, 21 bp, synthetic construct
  misc_feature 1..21: note = PCR Primer
  source 1..21: mol_type = other DNA
agcacaggtcaatatcttcac

SEQ ID NO: 124 - DNA, 21 bp, synthetic construct
  misc_feature 1..21: note = PCR Primer
  source 1..21: mol_type = other DNA
aatatagacccgcagatgatg

SEQ ID NO: 125 - DNA, 20 bp, synthetic construct
  misc_feature 1..20: note = PCR Primer
  source 1..20: mol_type = other DNA
tccgccaggattgattttcg

SEQ ID NO: 126 - DNA, 19 bp, synthetic construct
  misc_feature 1..19: note = PCR Primer
  source 1..19: mol_type = other DNA
ctccgggtatggagctatg

SEQ ID NO: 127 - DNA, 20 bp, synthetic construct
  misc_feature 1..20: note = PCR Primer
  source 1..20: mol_type = other DNA
gatcaggacgctcatattcg

SEQ ID NO: 128 - DNA, 20 bp, synthetic construct
  misc_feature 1..20: note = PCR Primer
  source 1..20: mol_type = other DNA
ctgtcatgtcggttagttcc

SEQ ID NO: 129 - DNA, 20 bp, synthetic construct
  misc_feature 1..20: note = PCR Primer
  source 1..20: mol_type = other DNA
acaggatgaaagtctttgcc

SEQ ID NO: 130 - DNA, 20 bp, synthetic construct
  misc_feature 1..20: note = PCR Primer
  source 1..20: mol_type = other DNA
gcaatttccgcttttgctcg

SEQ ID NO: 131 - DNA, 20 bp, synthetic construct
  misc_feature 1..20: note = PCR Primer
  source 1..20: mol_type = other DNA
aactgcttctcctcaccatc

SEQ ID NO: 132 - DNA, 18 bp, synthetic construct
  misc_feature 1..18: note = PCR Primer
  source 1..18: mol_type = other DNA
agaatcgtctggcggttg

SEQ ID NO: 133 - DNA, 19 bp, synthetic construct
  misc_feature 1..19: note = PCR Primer
  source 1..19: mol_type = other DNA
catcagaatcaatgctgcg

SEQ ID NO: 134 - DNA, 19 bp, synthetic construct
  misc_feature 1..19: note = PCR Primer
  source 1..19: mol_type = other DNA
tcgctgacttcagtttcgc

SEQ ID NO: 135 - DNA, 20 bp, synthetic construct
  misc_feature 1..20: note = PCR Primer
  source 1..20: mol_type = other DNA
gcctgccagatgatatggtc

SEQ ID NO: 136 - DNA, 19 bp, synthetic construct
  misc_feature 1..19: note = PCR Primer
  source 1..19: mol_type = other DNA
accagaccgtggttgttag

SEQ ID NO: 137 - DNA, 25 bp, synthetic construct
  misc_feature 1..25: note = PCR Primer
  source 1..25: mol_type = other DNA
tttgttatccagccatgatgttttc

SEQ ID NO: 138 - DNA, 25 bp, synthetic construct
  misc_feature 1..25: note = PCR Primer
  source 1..25: mol_type = other DNA
ttcctgtatacctgaaacgacaatg

SEQ ID NO: 139 - DNA, 20 bp, synthetic construct
  misc_feature 1..20: note = PCR Primer
  source 1..20: mol_type = other DNA
cacgcacatacaacggaggg

SEQ ID NO: 140 - DNA, 20 bp, synthetic construct
  misc_feature 1..20: note = PCR Primer
  source 1..20: mol_type = other DNA
ttgactgtgcgcaacatccc

SEQ ID NO: 141 - DNA, 18 bp, synthetic construct
  misc_feature 1..18: note = PCR Primer
  source 1..18: mol_type = other DNA
cctattccgcccatgacc

SEQ ID NO: 142 - DNA, 20 bp, synthetic construct
  misc_feature 1..20: note = PCR Primer
  source 1..20: mol_type = other DNA
caaaggtccaggcttttggg

SEQ ID NO: 143 - DNA, 20 bp, Escherichia coli
  source 1..20: mol_type = other DNA
catccatatcaccacgtcaa

SEQ ID NO: 144 - DNA, 20 bp, Escherichia coli
  source 1..20: mol_type = other DNA
ggtggcatcgcccacgtcaa

SEQ ID NO: 145 - DNA, 20 bp, Escherichia coli
  source 1..20: mol_type = other DNA
gtccattagcaccacgtcaa

SEQ ID NO: 146 - DNA, 20 bp, Escherichia coli
  source 1..20: mol_type = other DNA
tgcaatcgccttcacgtcaa

SEQ ID NO: 147 - DNA, 20 bp, Escherichia coli
  source 1..20: mol_type = other DNA
atagcggtaggccacgtcaa

SEQ ID NO: 148 - DNA, 20 bp, Escherichia coli
  source 1..20: mol_type = other DNA
tgggcgctgttgcacgtcaa

SEQ ID NO: 149 - DNA, 20 bp, Escherichia coli
  source 1..20: mol_type = other DNA
accaccgcgtaccacgtcaa

SEQ ID NO: 150 - DNA, 20 bp, Escherichia coli
  source 1..20: mol_type = other DNA
agccactccactggagacga

SEQ ID NO: 151 - DNA, 20 bp, Escherichia coli
  source 1..20: mol_type = other DNA
gatcttgtgaccggagacga

SEQ ID NO: 152 - DNA, 20 bp, Escherichia coli
  source 1..20: mol_type = other DNA
cccgttttgccaggagacga

SEQ ID NO: 153 - DNA, 20 bp, Escherichia coli
  source 1..20: mol_type = other DNA
catctctgctctggagacga

SEQ ID NO: 154 - DNA, 20 bp, Escherichia coli
  source 1..20: mol_type = other DNA
accacgtataccagccgctg

SEQ ID NO: 155 - DNA, 20 bp, Escherichia coli
  source 1..20: mol_type = other DNA
aattatctccacagccgctg

SEQ ID NO: 156 - DNA, 20 bp, Escherichia coli
  source 1..20: mol_type = other DNA
gaagtgttgatgagccgctg

SEQ ID NO: 157 - DNA, 20 bp, Escherichia coli
  source 1..20: mol_type = other DNA
aaaagccgaggcagccgctg

SEQ ID NO: 158 - DNA, 20 bp, Escherichia coli
  source 1..20: mol_type = other DNA
aacgttgaagtcagccgctg

SEQ ID NO: 159 - DNA, 20 bp, Escherichia coli
  source 1..20: mol_type = other DNA
cagagcaggttcagccgctg

SEQ ID NO: 160 - DNA, 20 bp, Escherichia coli
  source 1..20: mol_type = other DNA
catgccaatatcagccgctg

SEQ ID NO: 161 - DNA, 20 bp, Escherichia coli
  source 1..20: mol_type = other DNA
tctgctgctgccagccgctg

SEQ ID NO: 162 - DNA, 20 bp, Escherichia coli
  source 1..20: mol_type = other DNA
aagaagagatcaagccgctg

SEQ ID NO: 163 - DNA, 20 bp, Escherichia coli
  source 1..20: mol_type = other DNA
gacaacagtcttagccgctg

SEQ ID NO: 164 - DNA, 20 bp, Escherichia coli
  source 1..20: mol_type = other DNA
agacatacagccagccgctg

SEQ ID NO: 165 - DNA, 20 bp, Escherichia coli
  source 1..20: mol_type = other DNA
ttactaaaaaaaagccgctg

SEQ ID NO: 166 - DNA, 20 bp, Escherichia coli
  source 1..20: mol_type = other DNA
gtgtaaaaaacgagccgctg

SEQ ID NO: 167 - DNA, 20 bp, Escherichia coli
  source 1..20: mol_type = other DNA
cgcatagctggaagccgctg

SEQ ID NO: 168 - DNA, 20 bp, Escherichia coli
  source 1..20: mol_type = other DNA
aattagtggtgcagccgctg

SEQ ID NO: 169 - DNA, 20 bp, Escherichia coli
  source 1..20: mol_type = other DNA
tgctggtctttgagccgctg

SEQ ID NO: 170 - DNA, 20 bp, Escherichia coli
  source 1..20: mol_type = other DNA
ttccggatagttagccgctg

SEQ ID NO: 171 - DNA, 20 bp, Escherichia coli
  source 1..20: mol_type = other DNA
agcatatcgtgcagccgctg

SEQ ID NO: 172 - DNA, 20 bp, Escherichia coli
  source 1..20: mol_type = other DNA
ggcgggttttttagccgctg

SEQ ID NO: 173 - DNA, 20 bp, Escherichia coli
  source 1..20: mol_type = other DNA
cggctcgctcgcagccgctg

SEQ ID NO: 174 - DNA, 20 bp, Escherichia coli
  source 1..20: mol_type = other DNA
ctgcgattcctcagccgctg

SEQ ID NO: 175 - DNA, 20 bp, Escherichia coli
  source 1..20: mol_type = other DNA
acacgtcgatatagccgctg

SEQ ID NO: 176 - DNA, 20 bp, Escherichia coli
  source 1..20: mol_type = other DNA
gtgccactgattgcctttct

SEQ ID NO: 177 - DNA, 20 bp, Escherichia coli
  source 1..20: mol_type = other DNA
gtacctgtatttgcctttct

SEQ ID NO: 178 - DNA, 20 bp, Escherichia coli
  source 1..20: mol_type = other DNA
ttatcggcctgagcctttct

SEQ ID NO: 179 - DNA, 20 bp, Escherichia coli
  source 1..20: mol_type = other DNA
cttaacgtccgcgcctttct

SEQ ID NO: 180 - DNA, 20 bp, Escherichia coli
  source 1..20: mol_type = other DNA
ctgaccgataatgcctttct

SEQ ID NO: 181 - protein, 10 aa, synthetic construct
  REGION 1..10: note = Synthetic Sequence - protein degradation tag
ANDENYALAA

SEQ ID NO: 182 - protein, 11 aa, synthetic construct
  REGION 1..11: note = Synthetic Sequence - protein degradation tag
AANDENYALAA

SEQ ID NO: 183 - protein, 10 aa, synthetic construct
  REGION 1..10: note = Synthetic Sequence - protein degradation tag
GKQNNLSLAA

SEQ ID NO: 184 - protein, 11 aa, synthetic construct
  REGION 1..11: note = Synthetic Sequence - protein degradation tag
GKSNNNFALAA

SEQ ID NO: 185 - protein, 11 aa, synthetic construct
  REGION 1..11: note = protein degradation tag
GKENNNFALAA

SEQ ID NO: 186 - protein, 14 aa, synthetic construct
  REGION 1..14: note = Synthetic Sequence - protein degradation tag
GKTNSFNQNVALAA

SEQ ID NO: 187 - protein, 11 aa, synthetic construct
  REGION 1..11: note = Synthetic Sequence - protein degradation tag
GKSNQNLALAA

SEQ ID NO: 188 - protein, 9 aa, synthetic construct
  REGION 1..9: note = Synthetic Sequence - protein degradation tag
GKQNYALAA

SEQ ID NO: 189 - protein, 10 aa, synthetic construct
  REGION 1..10: note = Synthetic Sequence - protein degradation tag
ANDDNYALAA

SEQ ID NO: 190 - protein, 12 aa, synthetic construct
  REGION 1..12: note = Synthetic Sequence - protein degradation tag
ANDDQYGAALAA

SEQ ID NO: 191 - protein, 14 aa, synthetic construct
  REGION 1..14: note = Synthetic Sequence - protein degradation tag
ANDENYGQEFALAA

SEQ ID NO: 192 - protein, 13 aa, synthetic construct
  REGION 1..13: note = Synthetic Sequence - protein degradation tag
ANDETYGDYALAA

SEQ ID NO: 193 - protein, 13 aa, synthetic construct
  REGION 1..13: note = Synthetic Sequence - protein degradation tag
ANDETYGEYALAA

SEQ ID NO: 194 - protein, 15 aa, synthetic construct
  REGION 1..15: note = Synthetic Sequence - protein degradation tag
ANDETYGEETYALAA

SEQ ID NO: 195 - protein, 14 aa, synthetic construct
  REGION 1..14: note = Synthetic Sequence - protein degradation tag
ANDENYGAEYKLAA

SEQ ID NO: 196 - protein, 12 aa, synthetic construct
  REGION 1..12: note = Synthetic Sequence - protein degradation tag
ANDENYGAQLAA

SEQ ID NO: 197 - protein, 11 aa, synthetic construct
  REGION 1..11: note = Synthetic Sequence - protein degradation tag
AKNTNSYALAA

SEQ ID NO: 198 - protein, 11 aa, synthetic construct
  REGION 1..11: note = Synthetic Sequence - protein degradation tag
AKNTNSYAVAA

SEQ ID NO: 199 - protein, 11 aa, synthetic construct
  REGION 1..11: note = Synthetic Sequence - protein degradation tag
AKNNTTYALAA

SEQ ID NO: 200 - protein, 11 aa, synthetic construct
  REGION 1..11: note = Synthetic Sequence - protein degradation tag
AKNTNTYALAA

SEQ ID NO: 201 - protein, 11 aa, synthetic construct
  REGION 1..11: note = Synthetic Sequence - protein degradation tag
AKNNTSYALAA

SEQ ID NO: 202 - DNA, 52 bp, Escherichia coli
  source 1..52: mol_type = other DNA
gcgtggtgaaacatctgcaactgcgtaacaacagcttcggcggcgtggacat

SEQ ID NO: 203 - DNA, 52 bp, Escherichia coli
  source 1..52: mol_type = other DNA
atgtccacgccgccgaagctgttgttacgcagttgcagatgtttcaccacgc

SEQ ID NO: 204 - protein, 16 aa, Escherichia coli
VVKHLQLRNNSFGGVD

SEQ ID NO: 205 - DNA, 52 bp, Escherichia coli
  source 1..52: mol_type = other DNA
gcgtggtgaaacatctgtaattgcgtaacaacagcttcggcggcgtggacat

SEQ ID NO: 206 - DNA, 52 bp, Escherichia coli
  source 1..52: mol_type = other DNA
gcgtggtgaaacatctgtaactgcgtaacaacagcttcggcggcgtggacat

SEQ ID NO: 207 - protein, 5 aa, Escherichia coli
VVKHL

SEQ ID NO: 208 - DNA, 50 bp, Escherichia coli
  source 1..50: mol_type = other DNA
cttcggttccagccagctgtctcagtttatggaccagaacaacccgctgt

SEQ ID NO: 209 - DNA, 50 bp, Escherichia coli
  source 1..50: mol_type = other DNA
acagcgggttgttctggtccataaactgagacagctggctggaaccgaag

SEQ ID NO: 210 - protein, 16 aa, Escherichia coli
FGSSQLSQFMDQNNPL

SEQ ID NO: 211 - DNA, 50 bp, Escherichia coli
  source 1..50: mol_type = other DNA
cttcggttccagccagctgtctcagtttatgaaccagaacaacccgctgt

SEQ ID NO: 212 - protein, 16 aa, Escherichia coli
FGSSQLSQFMNQNNPL

SEQ ID NO: 213 - DNA, 50 bp, Escherichia coli
  source 1..50: mol_type = other DNA
cttcggttccagccagctgtctcagtttataaaccagaacaacccgctgt

SEQ ID NO: 214 - protein, 16 aa, Escherichia coli
FGSSQLSQFINQNNPL

SEQ ID NO: 215 - DNA, 51 bp, Escherichia coli
  source 1..51: mol_type = other DNA
acattaagttcaccgtcgccagcatcaccgatatagatatagcggaaagct

SEQ ID NO: 216 - DNA, 51 bp, Escherichia coli
  source 1..51: mol_type = other DNA
gtgaaaggcaaaaaaacggcctcccgatagggaagccgtagcaaagtgcgc

SEQ ID NO: 217 - DNA, 51 bp, Escherichia coli
  source 1..51: mol_type = other DNA
ggtggaccgattggtggcatgatttcagataagatcctgaaatcgccaagt

SEQ ID NO: 218 - DNA, 30 bp, Escherichia coli
  source 1..30: mol_type = other DNA
ctatcaatgggctaactacgttcgtggcgt

SEQ ID NO: 219 - DNA, 30 bp, Escherichia coli; identical to SEQ ID NO: 218
  source 1..30: mol_type = other DNA

SEQ ID NO: 220 - DNA, 32 bp, Escherichia coli
  source 1..32: mol_type = other DNA
attgcagcagctttatcatctgccgctggacg

SEQ ID NO: 221 - DNA, 32 bp, Escherichia coli; identical to SEQ ID NO: 220
  source 1..32: mol_type = other DNA

SEQ ID NO: 222 - DNA, 30 bp, Escherichia coli
  source 1..30: mol_type = other DNA
tctgcaactgcgtaacaacagcttcggcgg

SEQ ID NO: 223 - DNA, 30 bp, Escherichia coli; identical to SEQ ID NO: 222
  source 1..30: mol_type = other DNA

SEQ ID NO: 224 - DNA, 30 bp, Escherichia coli
  source 1..30: mol_type = other DNA
aaaccagtttgtaggctgtaactgcgggat

SEQ ID NO: 225 - DNA, 30 bp, Escherichia coli; identical to SEQ ID NO: 224
  source 1..30: mol_type = other DNA

SEQ ID NO: 226 - DNA, 20 bp, Escherichia coli
  source 1..20: mol_type = other DNA
tctctgtttgccaacgcatt

SEQ ID NO: 227 - DNA, 20 bp, Escherichia coli
  source 1..20: mol_type = other DNA
ccctgccactcacaccattc

SEQ ID NO: 228 - DNA, 20 bp, Escherichia coli
  source 1..20: mol_type = other DNA
actcacaccattcaggcgcc

SEQ ID NO: 229 - DNA, 20 bp, Escherichia coli
  source 1..20: mol_type = other DNA
cctggccgcgtgaatttgat

SEQ ID NO: 230 - DNA, 20 bp, Escherichia coli
  source 1..20: mol_type = other DNA
ctgccatcacgcgaacttta

SEQ ID NO: 231 - DNA, 20 bp, Escherichia coli
  source 1..20: mol_type = other DNA
tgcgacaatgggcgcatcga

SEQ ID NO: 232 - DNA, 20 bp, Escherichia coli
  source 1..20: mol_type = other DNA
tcgcacatgaaaactatcaa

SEQ ID NO: 233 - DNA, 20 bp, Escherichia coli
  source 1..20: mol_type = other DNA
caatgggctaactacgttcg

SEQ ID NO: 234 - DNA, 21 bp, Escherichia coli
  source 1..21: mol_type = other DNA
tcaatgggctaactacgttcg

SEQ ID NO: 235 - DNA, 20 bp, Escherichia coli
  source 1..20: mol_type = other DNA
caactgcgtaacaacagctt

SEQ ID NO: 236 - DNA, 21 bp, Escherichia coli
  source 1..21: mol_type = other DNA
gcaactgcgtaacaacagctt

SEQ ID NO: 237 - DNA, 20 bp, Escherichia coli
  source 1..20: mol_type = other DNA
gaccgcgacttccagtgaag

SEQ ID NO: 238 - DNA, 20 bp, Escherichia coli
  source 1..20: mol_type = other DNA
gcagctttatcatctgccgc

SEQ ID NO: 239 - DNA, 23 bp, Escherichia coli
  source 1..23: mol_type = other DNA
gcagcagctttatcatctgccgc

SEQ ID NO: 240 - DNA, 20 bp, Escherichia coli
  source 1..20: mol_type = other DNA
acaaatcgcgcttaacggtc

SEQ ID NO: 241 - DNA, 20 bp, Escherichia coli
  source 1..20: mol_type = other DNA
cagtttgtaggctgtaactg

SEQ ID NO: 242 - DNA, 21 bp, Escherichia coli
  source 1..21: mol_type = other DNA
ccagtttgtaggctgtaactg

SEQ ID NO: 243 - DNA, 20 bp, Escherichia coli
  source 1..20: mol_type = other DNA
gatcagctaatttccgcgct

SEQ ID NO: 244 - DNA, 35 bp, Escherichia coli
  source 1..35: mol_type = other DNA
gaaacaggccccccccatcgagcagaaaacggtgg

SEQ ID NO: 245 - DNA, 35 bp, Escherichia coli
  source 1..35: mol_type = other DNA
acaggccccccccatcgagcagaaaacggtggtgg

SEQ ID NO: 246 - DNA, 35 bp, Escherichia coli
  source 1..35: mol_type = other DNA
ggccccccccatcgagcagaaaacggtggtggatg

SEQ ID NO: 247 - DNA, 34 bp, Escherichia coli
  source 1..34: mol_type = other DNA
caaccgcccaacgcgttgggccgggggggaagcg

SEQ ID NO: 248 - DNA, 34 bp, Escherichia coli
  source 1..34: mol_type = other DNA
cgcttcccccccggcccaacgcgttgggcggttg

SEQ ID NO: 249 - DNA, 34 bp, Escherichia coli
  source 1..34: mol_type = other DNA
ggtccacgcgcgttcgaagtggggggggaggttc

SEQ ID NO: 250 - DNA, 34 bp, Escherichia coli
  source 1..34: mol_type = other DNA
gaacctccccccccacttcgaacgcgcgtggacc

SEQ ID NO: 251 - DNA, 29 bp, Escherichia coli
  source 1..29: mol_type = other DNA
cttccgcttcactggaagtcgcggtcgga

SEQ ID NO: 252 - DNA, 29 bp, Escherichia coli
  source 1..29: mol_type = other DNA
tccgaccgcgacttccagtgaagcggaag

SEQ ID NO: 253 - DNA, 29 bp, Escherichia coli
  source 1..29: mol_type = other DNA
cttccgcttcactggaagtcacaatcgga

SEQ ID NO: 254 - DNA, 29 bp, Escherichia coli
  source 1..29: mol_type = other DNA
gcagcagctttatcatctgccgctggacg

SEQ ID NO: 255 - DNA, 29 bp, Escherichia coli
  source 1..29: mol_type = other DNA
cgtccagcggcagatgataaagctgctgc

SEQ ID NO: 256 - DNA, 29 bp, Escherichia coli
  source 1..29: mol_type = other DNA
gcagtagttttatcatctgccgctggacg

SEQ ID NO: 257 - DNA, 30 bp, Escherichia coli; identical to SEQ ID NO: 224
  source 1..30: mol_type = other DNA

SEQ ID NO: 258 - DNA, 30 bp, Escherichia coli
  source 1..30: mol_type = other DNA
atcccgcagttacagcctacaaactggttt

SEQ ID NO: 259 - DNA, 30 bp, Escherichia coli
  source 1..30: mol_type = other DNA
aaattagtttgtaggctgtaactgcgggat

SEQ ID NO: 260 - DNA, 68 bp, Escherichia coli
  source 1..68: mol_type = other DNA
ttcttccgcttcactggaagtcgcggtcggaaccgtattgcagcagctttatcatctgccgctggacg

SEQ ID NO: 261 - DNA, 68 bp, Escherichia coli
  source 1..68: mol_type = other DNA
cgtccagcggcagatgataaagctgctgcaatacggttccgaccgcgacttccagtgaagcggaagaa

SEQ ID NO: 262 - DNA, 31 bp, Escherichia coli
  source 1..31: mol_type = other DNA
gaaaaccagtttgtaggctgtaactgcggga

SEQ ID NO: 263 - DNA, 31 bp, Escherichia coli
  source 1..31: mol_type = other DNA
tcccgcagttacagcctacaaactggttttc

SEQ ID NO: 264 - DNA, 68 bp, Escherichia coli
  source 1..68: mol_type = other DNA
ttcttccgcttcactggaagtcacaatcggaaccgtattgcagtagctttatcatctgccgctggacg

SEQ ID NO: 265 - DNA, 68 bp, Escherichia coli
  source 1..68: mol_type = other DNA
ttcttccgcttcactgaaagtcacaatcggaaccgtattgcagtagttttatcatctgccgctggacg

SEQ ID NO: 266 - DNA, 68 bp, Escherichia coli
  source 1..68: mol_type = other DNA
ttcttccgcttcactggaagtcgcaatcggaaccgtattgcagtagttttatcatctgccgctggacg

SEQ ID NO: 267 - DNA, 68 bp, Escherichia coli
  source 1..68: mol_type = other DNA
ttcttccgcttcactggaagtcacaatcggaaccgtattgcagtagttttatcatctgccgctggacg

SEQ ID NO: 268 - DNA, 68 bp, Escherichia coli
  source 1..68: mol_type = other DNA
ttcttccgcttcactggaagtcacaatcggaaccgtattgcagcagctttatcatctgccgctggacg

SEQ ID NO: 269 - DNA, 31 bp, Escherichia coli
  source 1..31: mol_type = other DNA
gaaaactagtttgtaggctgtaactgcggga

SEQ ID NO: 270 - DNA, 31 bp, Escherichia coli
  source 1..31: mol_type = other DNA
gaaaattagtttgtaggctgtaactgcggga

SEQ ID NO: 271 - DNA, 50 bp, Escherichia coli
  source 1..50: mol_type = other DNA
caacgcatttggctaccctgccactcacaccattcaggcgcctggccgcg

SEQ ID NO: 272 - DNA, 50 bp, Escherichia coli
  source 1..50: mol_type = other DNA
cgcggccaggcgcctgaatggtgtgagtggcagggtagccaaatgcgttg

SEQ ID NO: 273 - DNA, 50 bp, Escherichia coli
  source 1..50: mol_type = other DNA
caacgcatttggctattttgttactcacaccattcaggcgcctggccgcg

SEQ ID NO: 274 - DNA, 50 bp, Escherichia coli
  source 1..50: mol_type = other DNA
caacgcatttggctactttgtcactcacaccattcaggcgcctggccgcg

SEQ ID NO: 275 - DNA, 50 bp, Escherichia coli
  source 1..50: mol_type = other DNA
caacgcatttggctactttgccactcacaccattcaggcgcctggccgcg

SEQ ID NO: 276 - DNA, 50 bp, Escherichia coli
  source 1..50: mol_type = other DNA
caacgcatttggctattttgtcactcacaccattcaggcgcctggccgcg

SEQ ID NO: 277 - DNA, 50 bp, Escherichia coli
  source 1..50: mol_type = other DNA
ggtgctttgttacctgaagttgcgaaagcgtggggtatggcgacggtgcc

SEQ ID NO: 278 - DNA, 50 bp, Escherichia coli
  source 1..50: mol_type = other DNA
ggcaccgtcgccataccccacgctttcgcaacttcaggtaacaaagcacc

SEQ ID NO: 279 - DNA, 50 bp, Escherichia coli
  source 1..50: mol_type = other DNA
ggtgctttgttacctgaagttgcgaaagcataaagtatggcgacggtgcc

SEQ ID NO: 280 - DNA, 50 bp, Escherichia coli
  source 1..50: mol_type = other DNA
ggtgctttgttacctgaagttgcgaaagcgtaaggtatggcgacggtgcc

SEQ ID NO: 281 - DNA, 50 bp, Escherichia coli
  source 1..50: mol_type = other DNA
ggtgctttgttacctgaagttgcgaaagcgtaaaatatggcgacggtgcc

SEQ ID NO: 282 - DNA, 50 bp, Escherichia coli
  source 1..50: mol_type = other DNA
ggtgctttgttacctgaagttgcgaaagcgtaaagtatggcgacggtgcc

SEQ ID NO: 283 - DNA, 50 bp, Escherichia coli
  source 1..50: mol_type = other DNA
ggtgctttgttacctgaagttgcgaaagcataaaatatggcgacggtgcc

SEQ ID NO: 284 - DNA, 50 bp, Escherichia coli
  source 1..50: mol_type = other DNA
aaatccgtccagccagccgatggccgagctgtggatgggcgcacatccga

SEQ ID NO: 285 - DNA, 50 bp, Escherichia coli
  source 1..50: mol_type = other DNA
tcggatgtgcgcccatccacagctcggccatcggctggctggacggattt

SEQ ID NO: 286 - DNA, 50 bp, Escherichia coli
  source 1..50: mol_type = other DNA
aaatccgtccagccagccgatggccgaactataaatgggcgcacatccga

SEQ ID NO: 287 - DNA, 50 bp, Escherichia coli
  source 1..50: mol_type = other DNA
aaatccgtccagccagccgatggccaaactataaatgggcgcacatccga

SEQ ID NO: 288 - DNA, 50 bp, Escherichia coli
  source 1..50: mol_type = other DNA
aaatccgtccagccagccgatggccgagctataaatgggcgcacatccga

SEQ ID NO: 289 - DNA, 50 bp, Escherichia coli
  source 1..50: mol_type = other DNA
tgatccggcgaagctgcaagaatccagcccgctgccggttctcggcgctg

SEQ ID NO: 290 - DNA, 50 bp, Escherichia coli
  source 1..50: mol_type = other DNA
cagcgccgagaaccggcagcgggctggattcttgcagcttcgccggatca

SEQ ID NO: 291 - DNA, 50 bp, Escherichia coli
  source 1..50: mol_type = other DNA
tgatccggcgaagctgtaagaatccagcccgctgccggttctcggcgctg

SEQ ID NO: 292 - DNA, 50 bp, Escherichia coli
  source 1..50: mol_type = other DNA
gcggctgatatcgttctgcaggctgctatcgctgccggtgctccgaaaga

SEQ ID NO: 293 - DNA, 50 bp, Escherichia coli
  source 1..50: mol_type = other DNA
tctttcggagcaccggcagcgatagcagcctgcagaacgatatcagccgc

SEQ ID NO: 294 - DNA, 50 bp, Escherichia coli
  source 1..50: mol_type = other DNA
gcggctgatatcgttttgtaggctgctatcgctgccggtgctccgaaaga

SEQ ID NO: 295 - DNA, 50 bp, Escherichia coli; identical to SEQ ID NO: 294
  source 1..50: mol_type = other DNA

SEQ ID NO: 296 - DNA, 50 bp, Escherichia coli
  source 1..50: mol_type = other DNA
gcggctgatatcgttttgtaggctgttatcgctgccggtgctccgaaaga

SEQ ID NO: 297 - DNA, 50 bp, Escherichia coli
  source 1..50: mol_type = other DNA
gcggctgatattgttttgtaggctgctatcgctgccggtgctccgaaaga

SEQ ID NO: 298 - DNA, 51 bp, Escherichia coli
  source 1..51: mol_type = other DNA
cgcggtactgaaaactcagggtgctgcggcattcgaaggtgcggttatcgc

SEQ ID NO: 299 - DNA, 51 bp, Escherichia coli
  source 1..51: mol_type = other DNA
gcgataaccgcaccttcgaatgccgcagcaccctgagttttcagtaccgcg

SEQ ID NO: 300 - DNA, 51 bp, Escherichia coli
  source 1..51: mol_type = other DNA
cgcggtactgaaaacttagggtgctgcggcattcgaaggtgcggttatcgc

SEQ ID NO: 301 - DNA, 90 bp, Escherichia coli
  source 1..90: mol_type = other DNA
cgacgctggggcgtcttatgagcctgctgtcaccctttgacgtggtgatatggatgacggatggctggccgctgtatgaatcccgcctga

SEQ ID NO: 302 - protein, 30 aa, Escherichia coli
ATLGRLMSLLSPFDVVIWMTDGWPLYESRL

SEQ ID NO: 303 - DNA, 90 bp, Escherichia coli
  source 1..90: mol_type = other DNA
cgacgctggggcgtcttatgagcctgctgtcaccctttgacgtggtgatataaataacggatggctggccgctgtatgaatcccgcctga

SEQ ID NO: 304 - DNA, 90 bp, Escherichia coli
  source 1..90: mol_type = other DNA
cgacgctggggcgtcttatgagcctgctgtcaccctttgacgtggtaatataaataacggatggctggccgctgtatgaatcccgcctga

SEQ ID NO: 305 - DNA, 90 bp, Escherichia coli
  source 1..90: mol_type = other DNA
cgacgctggggcgtcttatgagcctgctgtcaccctttgacgtagtgatataaataacggatggctggccgctgtatgaatcccgcctga

SEQ ID NO: 306 - DNA, 90 bp, Escherichia coli
  source 1..90: mol_type = other DNA
ggagcggtggaacgccgcttcggcaacgatcttccgtcgtctccagtggagtggctgacggataatggttcatgctaccgggctaatgaa

SEQ ID NO: 307 - protein, 29 aa, Escherichia coli
GAVERRFGNDLPSSPVEWLTDNGSCYRAN

SEQ ID NO: 308 - DNA, 90 bp, Escherichia coli
  source 1..90: mol_type = other DNA
ggagcggtggaacgccgcttcggcaacgatcttccgtcgtctccagtgaaataactgacggataatggttcatgctaccgggctaatgaa

SEQ ID NO: 309 - DNA, 90 bp, Escherichia coli
  source 1..90: mol_type = other DNA
ggagcggtggaacgccgcttcggcaacgatcttccgtcgtctccagtggaataactaacggataatggttcatgctaccgggctaatgaa

SEQ ID NO: 310 - DNA, 90 bp, Escherichia coli
  source 1..90: mol_type = other DNA
ggagcggtggaacgccgcttcggcaacgatcttccgtcgtctccagtggaataactgacggataatggttcatgctaccgggctaatgaa

SEQ ID NO: 311 - DNA, 90 bp, Escherichia coli
  source 1..90: mol_type = other DNA
ggagcggtggaacgccgcttcggcaacgatcttccgtcgtctccagtaaaataactgacggataatggttcatgctaccgggctaatgaa

SEQ ID NO: 312 - DNA, 90 bp, Escherichia coli
  source 1..90: mol_type = other DNA
ggagcggtggaacgccgcttcggcaacgatcttccgtcgtctccagtgaaataactaacggataatggttcatgctaccgggctaatgaa

SEQ ID NO: 313 - DNA, 90 bp, Escherichia coli
  source 1..90: mol_type = other DNA
catcaaagcaatgtgccgcgtgctccgggtggcccgcagcggctggtatacgtggtgtcagcggcggacaaggataagcacgcgtcagca

SEQ ID NO: 314 - protein, 30 aa, Escherichia coli
IKAMCRVLRVARSGWYTWCGRRTRISTRQA

SEQ ID NO: 315 - DNA, 90 bp, Escherichia coli
  source 1..90: mol_type = other DNA
catcaaagcaatgtgccgcgtgctccgggtggcccgcagcggctggtatacataatgtcagcggcggacaaggataagcacgcgtcagca

SEQ ID NO: 316 - DNA, 90 bp, Escherichia coli; identical to SEQ ID NO: 315
  source 1..90: mol_type = other DNA

SEQ ID NO: 317 - DNA, 90 bp, Escherichia coli
  source 1..90: mol_type = other DNA
catcaaagcaatgtgccgcgtgctccgggtggcccgcagcggctggtatacatagtgtcagcggcggacaaggataagcacgcgtcagca

SEQ ID NO: 318 - DNA, 90 bp, Escherichia coli
  source 1..90: mol_type = other DNA
aagagcagcaacgcgatccggagatgcatcagaccaagaaaggcaatcagtggcactttggcatgaaggcccacattggtgtcgatgcca

SEQ ID NO: 319 - protein, 29 aa, Escherichia coli
KEQQRDPEMHQTKKGBQWHFGMKAHIGVD

SEQ ID NO: 320 - DNA, 90 bp, Escherichia coli
  source 1..90: mol_type = other DNA
aagagcagcaacgcgatccggagatgcatcagaccaagaaaggcaatcagtaacactttggcatgaaggcccacattggtgtcgatgcca

SEQ ID NO: 321 - DNA, 90 bp, Escherichia coli
  source 1..90: mol_type = other DNA
aagagcagcaacgcgatccggagatgcatcagaccaagaaaggcaatcaataacactttggcatgaaggcccacattggtgtcgatgcca

SEQ ID NO: 322 - DNA, 90 bp, Escherichia coli
  source 1..90: mol_type = other DNA
aagagcagcaacgcgatccggagatgcatcagaccaagaaaggcaatcagtgacactttggcatgaaggcccacattggtgtcgatgcca

SEQ ID NO: 323 - DNA, 90 bp, Escherichia coli
  source 1..90: mol_type = other DNA
aagagcagcaacgcgatccggaaatgcatcagaccaagaaaggcaatcaataacactttggcatgaaggcccacattggtgtcgatgcca

SEQ ID NO: 324 - protein, 5 aa, synthetic construct
  REGION 1..5: note = Synthetic Sequence - protein degradation tag
YAASV

SEQ ID NO: 325 - protein, 5 aa, synthetic construct
  REGION 1..5: note = Synthetic Sequence - protein degradation tag
YALAA

SEQ ID NO: 326 - DNA, 3684 bp, unidentified
  source 1..3684: mol_type = other DNA; note = Lachnospiraceae bacterium
  CDS 1..3684
atgagcaagcttgagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtcgtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagtctctgaagaagaacgacgccgtggtggccattatgaaggacctgctggattctgtgaagtctttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggtaccttcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcttccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 327 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 328 - DNA, 11174 bp, synthetic construct
  misc_feature 1..11174: note = Synthetic Sequence - synthetic oligonucleotide:Vector 3685
  misc_feature 70..573: note = CEN/ARS
  regulatory 610..714: note = promoter - Amp Rpromoter; regulatory_class = promoter
  gene 715..1575: note = AmpR
  rep_origin 1746..2334: note = ori
  regulatory 2853..3510: note = promoter - GAL1,10 promoter; regulatory_class = promoter
  protein_bind 3069..3186: bound_moiety = UAS
  gene 3541..7224: note = LbCpf1
  sig_peptide 7225..7272: note = nucleoplasmin NLS
  misc_feature 7282..7296: note = protein degradation tag
  regulatory 7318..7586: note = promoter - SNR52 promoter; regulatory_class = promoter
  misc_structure 7588..7606: note = Fn_crRNA
  regulatory 7631..7650: note = terminator - sup4 term; regulatory_class = terminator
  regulatory 7657: note = terminator - CYC1 terminator; regulatory_class = terminator
  regulatory 7680..7948: note = promoter - SNR52 promoter; regulatory_class = promoter
  misc_structure 7950..7968: note = Fn_crRNA
  regulatory 7993..8012: note = terminator - sup4 term; regulatory_class = terminator
  rep_origin 8246..8701: note = f1 ori
  regulatory 9001..9405: note = promoter - LEU2 promoter; regulatory_class = promoter
  gene 9418..10512: note = LEU2
  source 1..11174: mol_type = other DNA
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggcccgaattctctccttctcttaggtggcagagcaggtggagggtcgaccatactagtttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccatgggtttcgaatctagcatcatgagcaagcttgagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtcgtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagtctctgaagaagaacgacgccgtggtggccattatgaaggacctgctggattctgtgaagtctttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggtaccttcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcttccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggccaaaaagaaaaagggctctagctacgctgcatcagtctagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattggtgctggggttaccggcctgtttttttttgttttttatgtctaccggtcggccggtaccatcccgggtagatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattccaataacggaatccaacttgtttttttttgttttttatgtctaccggtcggccggtacccaattcgccctatagcctaggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 329 - DNA, 11174 bp, synthetic construct
  misc_feature 1..11174: note = Synthetic Sequence - synthetic oligonucleotide:Vector 3686
  misc_feature 70..573: note = CEN/ARS
  regulatory 610..714: note = promoter - AmpR promoter; regulatory_class = promoter
  gene 715..1575: note = AmpR
  rep_origin 1746..2334: note = ori
  regulatory 2853..3510: note = promoter - GAL1,10 promoter; regulatory_class = promoter
  protein_bind 3069..3186: bound_moiety = UAS
  gene 3541..7224: note = LbCpf1
  sig_peptide 7225..7272: note = nucleoplasmin NLS
  misc_feature 7282..7296: note = protein degradation tag
  regulatory 7318..7586: note = promoter - SNR52 promoter; regulatory_class = promoter
  misc_structure 7588..7606: note = Fn_crRNA
  regulatory 7631..7650: note = terminator - sup4 term; regulatory_class = terminator
  regulatory 7657: note = terminator - CYC1 terminator; regulatory_class = terminator
  regulatory 7680..7948: note = promoter - SNR52 promoter; regulatory_class = promoter
  misc_structure 7950..7968: note = Fn_crRNA
  regulatory 7993..8012: note = terminator - sup4 term; regulatory_class = terminator
  rep_origin 8246..8701: note = f1 ori
  regulatory 9001..9405: note = promoter - LEU2 promoter; regulatory_class = promoter
  gene 9418..10512: note = LEU2
  source 1..11174: mol_type = other DNA
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggcccgaattctctccttctcttaggtggcagagcaggtggagggtcgaccatactagtttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccatgggtttcgaatctagcatcatgagcaagcttgagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtcgtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagtctctgaagaagaacgacgccgtggtggccattatgaaggacctgctggattctgtgaagtctttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggtaccttcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcttccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggccaaaaagaaaaagggctctagctacgctctagctgcatagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattggtgctggggttaccggcctgtttttttttgttttttatgtctaccggtcggccggtaccatcccgggtagatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattccaataacggaatccaacttgtttttttttgttttttatgtctaccggtcggccggtacccaattcgccctatagcctaggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 330 - DNA, 11165 bp, synthetic construct
  misc_feature 1..11165: note = Synthetic Sequence - synthetic oligonucleotide:Vector 3687
  misc_feature 70..573: note = CEN/ARS
  regulatory 610..714: note = promoter - AmpR promoter; regulatory_class = promoter
  gene 715..1575: note = AmpR
  rep_origin 1746..2334: note = ori
  regulatory 2853..3510: note = promoter - GAL1,10 promoter; regulatory_class = promoter
  protein_bind 3069..3186: bound_moiety = UAS
  gene 3541..7224: note = LbCpf1
  sig_peptide 7225..7272: note = nucleoplasmin NLS
  misc_feature 7282..7287: note = protein degradation tag
  regulatory 7309..7577: note = promoter - SNR52 promoter; regulatory_class = promoter
  misc_structure 7579..7597: note = Fn_crRNA
  regulatory 7622..7641: note = terminator - sup4 term; regulatory_class = terminator
  regulatory 7648: note = terminator - CYC1 terminator; regulatory_class = terminator
  regulatory 7671..7939: note = promoter - SNR52 promoter; regulatory_class = promoter
  misc_structure 7941..7959: note = Fn_crRNA
  regulatory 7984..8003: note = terminator - sup4 term; regulatory_class = terminator
  rep_origin 8237..8692: note = f1 ori
  regulatory 8992..9396: note = promoter - LEU2 promoter; regulatory_class = promoter
  gene 9409..10503: note = LEU2
  source 1..11165: mol_type = other DNA
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggcccgaattctctccttctcttaggtggcagagcaggtggagggtcgaccatactagtttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccatgggtttcgaatctagcatcatgagcaagcttgagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtcgtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagtctctgaagaagaacgacgccgtggtggccattatgaaggacctgctggattctgtgaagtctttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggtaccttcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcttccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggccaaaaagaaaaagggctctagcacgcgttagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattggtgctggggttaccggcctgtttttttttgttttttatgtctaccggtcggccggtaccatcccgggtagatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactgttgtagattccaataacggaatccaacttgtttttttttgttttttatgtctaccggtcggccggtacccaattcgccctatagcctaggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 331 - DNA, 66 bp, Escherichia coli
  source 1..66: mol_type = other DNA
cttccgcttcactggaagtcgcggtcggaaccgtattgcagcagctttatcatctgccgctggacg

SEQ ID NO: 332 - DNA, 66 bp, Escherichia coli
  source 1..66: mol_type = other DNA
cgtccagcggcagatgataaagctgctgcaatacggttccgaccgcgacttccagtgaagcggaag

SEQ ID NO: 333 - DNA, 66 bp, Escherichia coli
  source 1..66: mol_type = other DNA
cttccgcttcactggaagtcacaatcggaaccgtattgcagtagctttatcatctgccgctggacg

SEQ ID NO: 334 - DNA, 66 bp, Escherichia coli
  source 1..66: mol_type = other DNA
cttccgcttcactgaaagtcacaatcggaaccgtattgcagtagttttatcatctgccgctggacg

SEQ ID NO: 335 - DNA, 66 bp, Escherichia coli
  source 1..66: mol_type = other DNA
cttccgcttcactggaagtcgcaatcggaaccgtattgcagtagttttatcatctgccgctggacg

SEQ ID NO: 336 - DNA, 66 bp, Escherichia coli
  source 1..66: mol_type = other DNA
cttccgcttcactggaagtcacaatcggaaccgtattgcagtagttttatcatctgccgctggacg

SEQ ID NO: 337 - DNA, 66 bp, Escherichia coli
  source 1..66: mol_type = other DNA
cttccgcttcactggaagtcacaatcggaaccgtattgcagcagctttatcatctgccgctggacg

SEQ ID NO: 338 - DNA, 50 bp, Escherichia coli
  source 1..50: mol_type = other DNA
gcggctgatatcgttttgaaggctgctatcgctgccggtgctccgaaaga

SEQ ID NO: 339 - DNA, 90 bp, Escherichia coli
  source 1..90: mol_type = other DNA
cgacgctggggcgtcttatgagcctgctgtcaccctttgacgtggtaatataaatgacggatggctggccgctgtatgaatcccgcctga

SEQ ID NO: 340 - DNA, 90 bp, Escherichia coli
  source 1..90: mol_type = other DNA
catcaaagcaatgtgccgcgtgctccgggtggcccgcagcggctagtatacataatgtcagcggcggacaaggataagcacgcgtcagca